(19) United States
(12) Patent Application Publication
(10) Pub. No.: US 2026/0355166 A1
(43) Pub. Date: Nov. 12, 2026

(54) 2-SUBSTITUTED PIPERIDINE DERIVATIVES, PREPARATION METHODS AND MEDICINAL USES THEREOF

(71) Applicants: JIANGSU HANSOH PHARMACEUTICAL GROUP CO., LTD., Lianyungang (CN); HANSOH BIO LLC, Rockville, MD (US); SHANGHAI HANSOH BIOMEDICAL CO., LTD., Shanghai (CN)

(72) Inventors: Avinash KHANNA, Rockville, MD (US); Matthew KIER, Rockville, MD (US); Hugh Y. ZHU, Rockville, MD (US)

(21) Appl. No.: 19/110,124

(22) PCT Filed: Sep. 11, 2023
(86) PCT No.: PCT/CN2023/117941

§ 371 Date: Mar. 10, 2025

Related U.S. Application Data
(60) Provisional application No. 63/375,224, filed on Sep. 10, 2022.

(51) Int. Cl. A61K 31/438; A61K 31/4747; C07D 401/06; C07D 471/10; C07D 491/107

(52) U.S. Cl. CPC A61K 31/438; A61K 31/4747; C07D 401/06; C07D 471/10; C07D 491/107

(57) ABSTRACT

Compounds of formula (I) as 2-substituted piperidine derivatives, the preparation method thereof, pharmaceutical compositions comprising the compounds, and the pharmaceutical uses for the treatment of a disease or disorder.

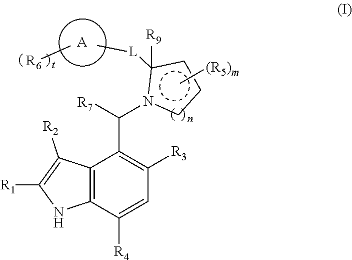

### FIELD OF THE INVENTION

The present invention belongs to the field of medicine, and relates to 2-substituted piperidine derivatives, preparation methods thereof, pharmaceutical compositions comprising the compounds, and medical uses thereof.

### BACKGROUND FOR INVENTION

The complement system is a part of the innate immunosurveillance, playing a critical role in eliminating pathogens and in the tissue homeostasis. The complement cascade can be activated by three different pathways including classical (CP), lectin (LP), and alternative pathway (AP). The CP and LP are initiated on target surfaces by immune complexes and binding of mannan-binding lectin or ficolin to a particular of microbial sugar moiety pattern, respectively. However, the AP does not require specific initiation. The AP cascade is initiated by spontaneous hydrolysis of C3 (tick-over) and subsequent deposition of C3b on an activating surface. The three complement activation pathways converge on two major events, C3 cleavage and C5 cleavage. C3 convertases split C3 into C3a and C3b. C3b forms additional AP C3 convertases (amplification) as well as C5 convertases. C5 convertases cleave C5 into C5a and C5b. The produced C5b initiates the formation of the C5b-9 membrane attack complex (MAC) with C6-C9, leading to lysis of bacteria and cells by insertion into a membrane. The split products C3a and C5a function as anaphylatoxins to promote pro-inflammatory responses through activation and chemotaxis of leukocytes. C3b also plays a key role in removing bacteria and cellular waste such as immune complexes and apoptotic cells through promoting phagocytosis by opsonization. (Front Immunol. 2015 Jun. 2; 6:262. doi: 10.3389/fimmu.2015.00262. eCollection 2015. Complement System Part I-Molecular Mechanisms of Activation and Regulation. Nicolas S Merle, Sarah Elizabeth Church, Veronique Fremeaux-Bacchi, Lubka T Roumenina). The AP maintains the basal complement activity through a “tick-over process. Moreover, the AP contributes more than 80% of terminal lysis pathway activation (MAC formation) through an amplification loop even if initiated via the other CP or LP. (Harboe, M., Garred, P., Karlstrøm, E., Lindstad, J. K., Stahl, G. L., Mollnes, T. E., 2009. The down-stream effects of mannan-induced lectin complement pathway activation depend quantitatively on alternative pathway amplification. Mol. Immunol. 47, 373-380. https://doi.org/10.1016/j.molimm.2009.09.005). The spontaneous activated C3 forms C3 convertase by binding with factor B (FB). After cleavage of FB into Bb by factor D, C3b and Bb generate the AP C3 convertase (C3bBb). The newly formed C3bBb cleaves more C3 to generate more AP C3 convertases, leading to the amplification of complement cascade. As the AP is ready to exert full complement activity within seconds, it can lead to normal tissue injury if not controlled properly. (J Clin Invest. 2020 May 1; 130 (5): 2152-2163. doi: 10.1172/JCI136094. Complementopathies and precision medicine. Eleni Gavriilaki, Robert A Brodsky). Dysregulated complement activation has been shown to be associated with diseases in various organs including paroxysmal nocturnal hemoglobinuria, age-related macular degeneration, rheumatoid arthritis, hemolytic uremic syndrome, myasthenia gravis, and C3 glomerulo-nephriti. (J Clin Invest. 2020 May 1; 130 (5): 2152-2163. doi: 10.1172/JCI136094.). Therefore, controlling the AP through FB inhibition may be a powerful strategy for limiting the overactivation of the Complement pathway.

Currently, there are no small-molecules approved for modulating the Complement pathways. Examples of Factor B inhibitors are described in the following disclosures: Advanced Vision Therapies Inc. patent publication WO2008/106644 titled “Treatment of diseases characterized by inflammation”; Wellstate Immunotherapeutics patent publication WO2012/151468 titled “Complement Factor B analogs and their uses”; William Marsh Rice University patent publication WO2014/035876 titled “Heat-inactivated Complement Factor B compositions and methods”; Muse. Foundation for Research Development patent publication US1999/023485 titled “Blocking factor b to treat complement-mediated immune disease”; and Novartis patent publication WO2013/192345 and US2015/126592 titled “Complement pathway modulators and uses thereof. Additional Factor B inhibitors are described in Novartis patent publications WO2015/066241, US2016/311779, WO2015/009616, US2016/152605, WO2014/143638, and US2016/024079. Another example of Factor B inhibitors is the IONIS Pharmaceuticals Inc. patent publication WO2015/038939 titled “Modulators of Complement Factor B”. Examples of granted patents covering Factor B inhibitors include U.S. Pat. Nos. 9,452,990; 9,676,728; 9,682,968; and 9,475,806.

Given the large array of diseases that are driven by an overactive complement pathway there is a high unmet need for patients of Complement diseases. This invention aims to provide compounds which modulate Factor B and treat disorders associated with the dysregulation of the Complement pathway.

### SUMMARY OF THE INVENTION

The present invention, in one aspect, provides a compound of formula (I), or a tautomer, cis- or trans-isomer, mesomer, racemate, enantiomer, diastereomer, or mixture thereof, or a pharmaceutically acceptable salt, solvate, or prodrug thereof,

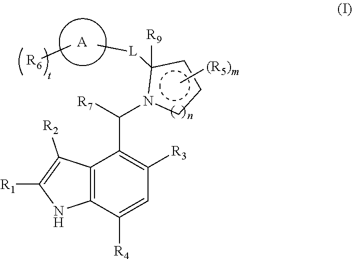

wherein,

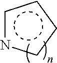

is a saturated or unsaturated ring;
A is cycloalkyl, heterocyclyl, aryl or heteroaryl;
L is bond, (CRₐR_{b})ₚ;
Rₐ and R_{b} are independently selected from the group consisting of hydrogen, deuterium, halogen, amino, cyano, hydroxy, alkyl, alkoxy, alkylthio, haloalkyl, hydroxyalkyl, cycloalkyl, heterocyclyl, aryl and heteroaryl;
R₁ and R₂ are independently selected from the group consisting of hydrogen, deuterium, halogen, amino, cyano, hydroxy, alkyl, alkoxy, alkylthio, haloalkyl and hydroxyalkyl;
R₃ and R₄ are independently selected from the group consisting of hydrogen, deuterium, halogen, amino, cyano, hydroxy, alkyl, alkoxy, alkylthio, haloalkyl, haloalkenyl, hydroxyalkyl, deuterated alkoxy, haloalkoxy, cycloalkyl, heterocyclyl, aryl, heteroaryl, cycloalkylxoy, heterocyclylxoy, arylxoy and heteroarylxoy, optionally the hydroxy, alkyl, alkoxy, alkylthio, haloalkyl, hydroxyalkyl, deuterated alkoxy, haloalkoxy, cycloalkyl, heterocyclyl, aryl, heteroaryl, cycloalkylxoy, heterocyclylxoy, arylxoy and heteroarylxoy substituted with one or more substituents selected from deuterium, halogen, amino, cyano, hydroxy, alkyl, alkoxy, alkylthio, haloalkyl, hydroxyalkyl, cycloalkyl, heterocyclyl, aryl and heteroaryl;
R₅ is independently selected from the group consisting of hydrogen, deuterium, halogen, amino, cyano, hydroxy, alkyl, alkenyl, alkynyl, alkoxy, alkylthio, haloalkyl, hydroxyalkyl, cycloalkyl, heterocyclyl, aryl and heteroaryl, optionally the amino, alkyl, alkoxy, alkylthio, haloalkyl, hydroxyalkyl, cycloalkyl, heterocyclyl, aryl and heteroaryl substituted with one or more substituents selected from deuterium, halogen, amino, cyano, hydroxy, alkyl, alkoxy, alkylthio, haloalkyl and hydroxyalkyl;
or, two of R₅ together with the C atom to which they are attached form a cycloalkyl or heterocyclyl, optionally the cycloalkyl or heterocyclyl substituted with one or more substituents selected from hydrogen, deuterium, halogen, amino, cyano, hydroxy, alkyl, alkoxy, alkylalkoxy, alkoxyalkyl, alkylthio, haloalkyl and hydroxyalkyl;
or, two of R₅ together with the C atom to which they are attached form a cycloalkyl or heterocyclyl, optionally the cycloalkyl or heterocyclyl substituted with one or more substituents selected from hydrogen, deuterium, halogen, amino, cyano, hydroxy, alkyl, alkoxy, alkylalkoxy, alkoxyalkyl, alkylthio, haloalkyl and hydroxyalkyl;
or, two of R₅ together with the C atom to which they are attached form a carbon-carbon double bond;
R₆ is selected from the group consisting of hydrogen, deuterium, halogen, amino, cyano, hydroxy, alkyl, alkoxy, alkylthio, haloalkyl, hydroxyalkyl, cycloalkyl, heterocyclyl, aryl, heteroaryl, —(CH₂)ᵣOR₈, —(CH₂)ᵣC(O)R₈, —S(O)NHalkyl, —SO₂ alkyl, —C(O)NHSO₂ alkyl and —SO₂NHC(O)alkyl;
R₇ is selected from the group consisting of hydrogen, deuterium, halogen, amino, cyano, hydroxy, alkyl, alkoxy, alkylthio, haloalkyl and hydroxyalkyl;
R₉ is selected from the group consisting of deuterium, halogen, amino, cyano, hydroxy, alkyl, alkoxy, alkylthio, haloalkyl, hydroxyalkyl, cycloalkyl and heterocyclyl;
or, R₉ taken together with one of R₆ form a saturated or unsaturated cycloalkyl or a saturated or unsaturated heterocyclyl, optionally the cycloalkyl or heterocyclyl is substituted with one or more substituents selected from hydrogen, deuterium, halogen, amino, cyano, hydroxy, alkyl, alkoxy, alkylalkoxy, alkoxyalkyl, alkylthio, haloalkyl and hydroxyalkyl, and another R₆ is selected from the group consisting of hydrogen, deuterium, halogen, amino, cyano, hydroxy, alkyl, alkoxy, alkylthio, haloalkyl, hydroxyalkyl, cycloalkyl, heterocyclyl, aryl, heteroaryl, —(CH₂)ᵣOR₈, —(CH₂)ᵣC(O)R₈, —S(O)NHalkyl, —SO₂ alkyl, —C(O)NHSO₂-alkyl and —SO₂NHC(O)alkyl;
R₈ is selected from the group consisting of deuterium, halogen, amino, cyano, hydroxy, alkyl, alkoxy, alkylthio, haloalkyl and hydroxyalkyl;
p is 1, 2 or 3;
tis 1, 2 or 3;
m is 1, 2 or 3; and
n is 0, 1, 2 or 3;
Provided that, R₃ and R₄ are not simultaneously methyl.

In some embodiments, A is C₆₋₁₀ aryl or 5-10 membered heteroaryl.

In preferred embodiments, A is phenyl, benzocycloalkyl, or 5-8 membered heteroaryl containing 1, 2 or 3 of N heteroatoms;

In more preferred embodiments, A is

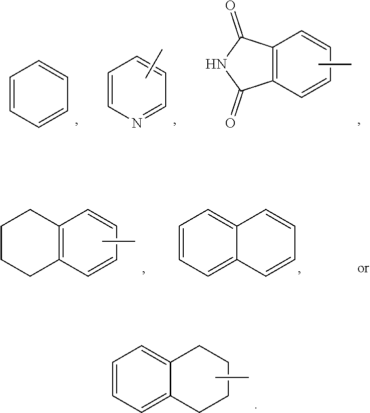

In some embodiments, L is bond, CH₂.

In some embodiments, wherein R₁ and R₂ are independently selected from hydrogen.

In some embodiments, wherein R₃ and R₄ are independently selected from the group consisting of hydrogen, deuterium, halogen, amino, cyano, hydroxy, C₁₋₆ alkyl, C₁₋₆ alkoxy, C₁₋₆ alkylthio, C₁₋₆ haloalkyl, C₁₋₆ haloalkenyl C₁₋₆ hydroxyalkyl, deuterated C₁₋₆ alkoxy, C₁₋₆ haloalkoxy, C₃₋₆ cycloalkyl, 4-10 membered heterocyclyl, C₆₋₁₀ aryl, 5-10 membered heteroaryl, C₃₋₆ cycloalkyloxy, 4-10 membered heterocyclyloxy, C₆₋₁₀ aryloxy and 5-10 membered heteroaryloxy, optionally the C₁₋₆ alkyl, C₁₋₆ alkoxy, C₁₋₆ alkylthio, C₁₋₆ haloalkyl, C₁₋₆ hydroxyalkyl, deuterated C₁₋₆ alkoxy, C₁₋₆ haloalkoxy substituted with one or more substituents selected from deuterium, halogen, amino, cyano, hydroxy, C₁₋₆ alkyl, C₁₋₆ alkoxy, C₁₋₆ alkylthio, C₁₋₆ haloalkyl, C₁₋₆ hydroxyalkyl, C₃₋₆ cycloalkyl, 4-10 membered heterocyclyl, C₆₋₁₀ aryl and 5-10 membered heteroaryl.

In preferred embodiments, R₃ and R₄ are independently selected from the group consisting of deuterium, halogen, C₁₋₃ alkyl, C₁₋₃ alkoxy, deuterated C₁₋₃ alkoxy, C₁₋₃ haloalkoxy, C₃₋₆ cycloalkyl and C36 cycloalkyloxy, optionally the C₁₋₃ alkyl, C₁₋₃ alkoxy, deuterated C₁₋₃ alkoxy, C₁₋₃ haloalkoxy substituted with one or more substituents selected from C₃₋₆ cycloalkyl, 4-6 membered heterocyclyl, C₆₋₁₀ aryl and 5-10 membered heteroaryl.

In some embodiments, R₅ is independently selected from the group consisting of hydrogen, deuterium, halogen, amino, cyano, hydroxy, C₁₋₆ alkyl, C₂₋₆ alkenyl, C₂₋₆ alkynyl, C₁₋₆ alkoxy, C₁₋₆ alkylthio, C₁₋₆ haloalkyl, C₁₋₆ hydroxyalkyl, C₃₋₈ cycloalkyl, 4-10 membered heterocyclyl, C₅₋₁₀ aryl and 5-10 membered heteroaryl, optionally the C₁₋₆ alkyl, C₁₋₆ alkoxy, C₁₋₆ alkylthio, C₁₋₆ haloalkyl, C₁₋₆ hydroxyalkyl, C₃₋₈ cycloalkyl, 4-10 membered heterocyclyl, C₅₋₁₀ aryl and 5-10 membered heteroaryl substituted with one or more substituents selected from deuterium, halogen, amino, cyano, hydroxy, C₁₋₆ alkyl, C₁₋₆ alkoxy, C₁₋₆ alkylthio, C₁₋₆ haloalkyl and C₁₋₆ hydroxyalkyl.

In some embodiments, two of R₅ together with the C atom to which they are attached form C₃₋₆ cycloalkyl or 4-6 membered heterocyclyl containing 1, 2 or 3 heteroatoms selected from N, O or S, optionally substituted with one or more substituents selected from hydrogen, deuterium, halogen, amino, cyano, hydroxy, C₁₋₆ alkyl, C₁₋₆ alkoxy, C₁₋₆ alkylC₁₋₆ alkoxy, C₁₋₆ alkoxyC₁₋₆ alkyl, C₁₋₆ alkylthio, C₁₋₆ haloalkyl and C₁₋₆ hydroxyalkyl.

In some embodiments, two of R₅ together with the C atom to which they are attached form a carbon-carbon double bond.

In some embodiments, R₆ is selected from the group consisting of hydrogen, deuterium, halogen, amino, cyano, hydroxy, C₁₋₆ alkyl, C₁₋₆ alkoxy, C₁₋₆ alkylthio, C₁₋₆ haloalkyl, C₁₋₆ hydroxyalkyl, C₃₋₈ cycloalkyl, 4-10 membered heterocyclyl, C₅₋₁₀ aryl and 5-10 membered heteroaryl, —(CH₂)ᵣC₁₋₆ alkoxy, —(CH₂), —C(O)OH, —S(O)NHC₁₋₆ alkyl, —SO₂C₁₋₆ alkyl, —C(O)NHSO₂C₁₋₆ alkyl and —SO₂NHC(O)C₁₋₆ alkyl.

In some embodiments, R₆ is —F, —OMe, —CH₂OH, —CH₂OCH₃, —CH₂F, —CF₂H, —CF₃, —COOH, —C(O)NHSO₂CH₃, —S(O)NHCH₃, or 5-6 membered heterocyclyl containing 1-3 of heteroatom selected from N, O and S, or 5-6 membered heteroaryl containing 1-3 of heteroatom selected from N, O and S.

In some embodiments, R₇ is hydrogen or C₁₋₃ alkyl.

In some embodiments, R₉ is selected from the group consisting of deuterium, halogen, amino, cyano, hydroxy, C₁₋₆ alkyl, C₁₋₆ alkoxy, C₁₋₆ alkylthio, C₁₋₆ haloalkyl, C₁₋₆ hydroxyalkyl, C₃₋₈ cycloalkyl and 4-10 membered heterocyclyl containing 1, 2 or 3 heteroatoms selected from N, O or S.

In preferred embodiments, R₉ is selected from the group consisting of deuterium, halogen, amino, cyano, hydroxy, C₁₋₃ alkyl, C₁₋₆ alkoxy, C₁₋₃ alkylthio, C₁₋₃ haloalkyl, C₁₋₃ hydroxyalkyl, C₃₋₆ cycloalkyl and 4-6 membered heterocyclyl containing 1, 2 or 3 heteroatoms selected from N, O or S.

In more preferred embodiments, R₉ is selected from the group consisting of deuterium, halogen, amino, cyano, hydroxy, methyl, ethyl, cyclopropyl, cyclobutyl.

In some embodiments, R₉ taken together with one of R₆ form a saturated or unsaturated C₃₋₇ cycloalkyl or a saturated or unsaturated 3-7 membered heterocyclyl containing 1, 2 or 3 heteroatoms selected from N, O or S, optionally the C₃₋₇ cycloalkyl or 3-7 membered heterocyclyl is substituted with 1 or 2 substituents selected from hydrogen, deuterium, halogen, amino, cyano, hydroxy, C₁₋₆ alkyl, C₁₋₆ alkoxy, C₁₋₆ alkylalkoxy, C₁₋₆ alkoxyalkyl, C₁₋₆ alkylthio, C₁₋₆ haloalkyl and C₁₋₆ hydroxyalkyl, and another R₆ is selected from the group consisting of hydrogen, deuterium, halogen, amino, cyano, hydroxy, C₁₋₃ alkyl, C₁₋₆ alkoxy, C₁₋₆ alkylthio, C₁₋₆ haloalkyl, C₁₋₆ hydroxyalkyl, C₃₋₈ cycloalkyl, 4-10 membered heterocyclyl, C₅₋₁₀ aryl and 5-10 membered heteroaryl, —(CH₂)ᵣC₁₋₆ alkoxy, —(CH₂)ᵣC(O)OH, —S(O)NHC₁₋₆ alkyl, —SO₂C₁₋₆ alkyl, —C(O)NHSO₂C₁₋₆ alkyl and —SO₂NHC(O)C₁₋₆ alkyl;

In more preferred embodiments, R₉ taken together with one of R₆ form a saturated or unsaturated C4-6 cycloalkyl or a saturated or unsaturated 4-7 membered heterocyclyl containing 1, 2 or 3 heteroatoms selected from N, O or S, optionally the saturated or unsaturated C4-6 cycloalkyl or saturated or unsaturated 4-7 membered heterocyclyl is substituted with 1 or 2 substituents selected from hydrogen, deuterium, halogen, amino, cyano, hydroxy, C₁₋₃ alkyl, C₁₋₃ alkoxy, C₁₋₃ alkylalkoxy, C₁₋₃ alkoxyalkyl, C₁₋₃ alkylthio, C₁₋₃ haloalkyl and C₁₋₃ hydroxyalkyl, and another R₆ is selected from the group consisting of F, —OMe, —CH₂OH, —CH₂OCH₃, —CH₂F, —CF₂H, —CF₃, —COOH, —C(O)NHSO₂CH₃, —S(O)NHCH₃.

In a preferred embodiment, the compound of formula (I) may be compounds of formula (II), or a tautomer, cis- or trans-isomer, mesomer, racemate, enantiomer, diastereomer, or mixture thereof, or a pharmaceutically acceptable salt, solvate, or prodrug thereof,

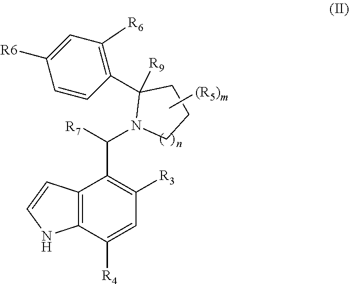

In a preferred embodiment, the compound of formula (I) may be compounds of formula (III-a)-(III-b), or a tautomer, cis- or trans-isomer, mesomer, racemate, enantiomer, diastereomer, or mixture thereof, or a pharmaceutically acceptable salt, solvate, or prodrug thereof,

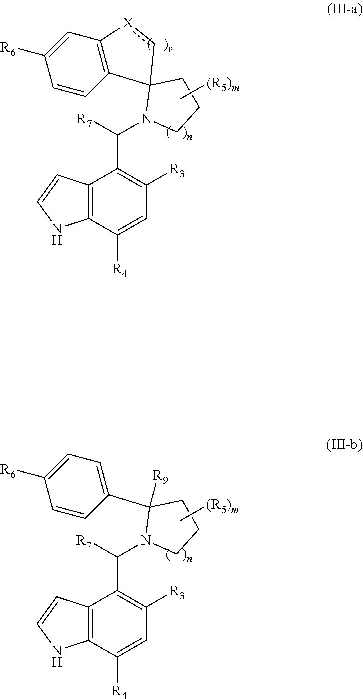

wherein,
X is CR_{c}R_{d}, NRₑ, O or S;
[symbol] is a single or double bond;
R_{c}, R_{d} and Rₑ are independently selected from the group consisting of hydrogen, deuterium, halogen, amino, cyano, hydroxy, C₁₋₆ alkyl, C₁₋₆ alkoxy, C₁₋₆ alkylthio, C₁₋₆ haloalkyl, C₁₋₆ hydroxyalkyl, C₃₋₈ cycloalkyl, C₃₋₈ heterocyclyl, C₆₋₁₀ aryl and C₆₋₁₀ heteroaryl;
preferably, R_{c}, R_{d} and Rₑ are independently selected from the group consisting of hydrogen, deuterium, halogen, amino, cyano, hydroxy, C₁₋₃ alkyl, C₁₋₃ alkoxy, C₁₋₃ alkylthio, C₁₋₃ haloalkyl, C₁₋₃ hydroxyalkyl, C₃₋₆ cycloalkyl, C₄₋₆ heterocyclyl, C₆₋₁₀ aryl and C₆₋₁₀ heteroaryl; preferably, R_{c}, R_{d} and Rₑ are independently selected from the group consisting of hydrogen, deuterium, halogen, amino, cyano, hydroxy, methyl, ethyl;
v is 0, 1, 2 or 3.

In a preferred embodiment, the compound of formula (I) may be compounds of formula (IV-a)-(IV-f), or a tautomer, cis- or trans-isomer, mesomer, racemate, enantiomer, diastereomer, or mixture thereof, or a pharmaceutically acceptable salt, solvate, or prodrug thereof,

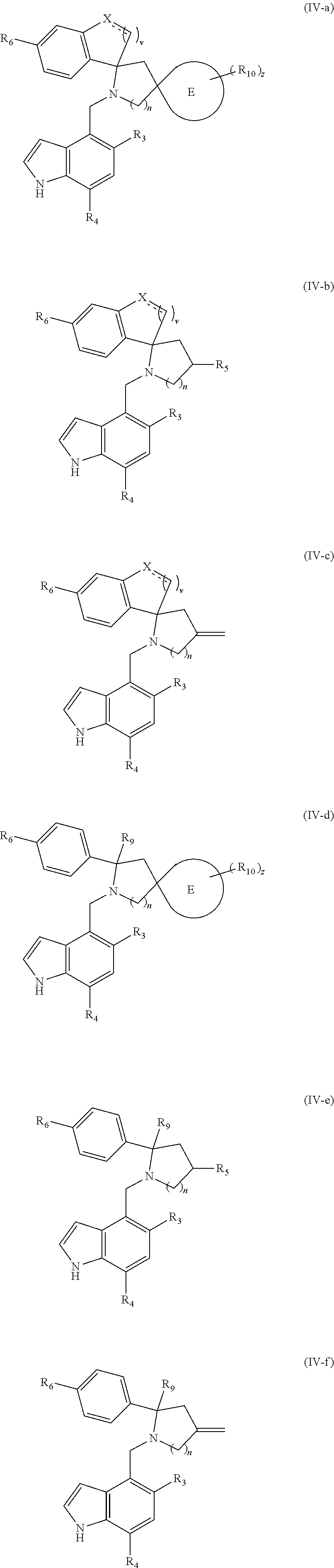

wherein,
E is

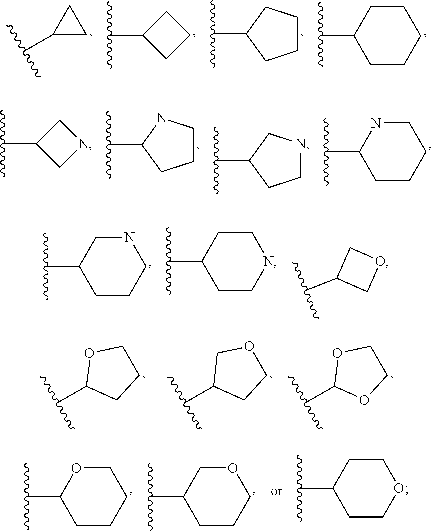

R₁₀ is selected from deuterium, halogen, amino, cyano, hydroxy, C₁₋₃ alkyl, C₂₋₄ alkenyl, C₂₋₄ alkynyl, C₁₋₃ alkoxy, C₁₋₃ alkylC₁₋₃ alkoxy, C₁₋₃ alkoxyC₁₋₃ alkyl, C₁₋₃alkylthio, C₁₋₃ haloalkyl and C₁₋₃ hydroxyalkyl;
n is 1; and,
z is 0, 1, 2 or 3.

In a preferred embodiment,

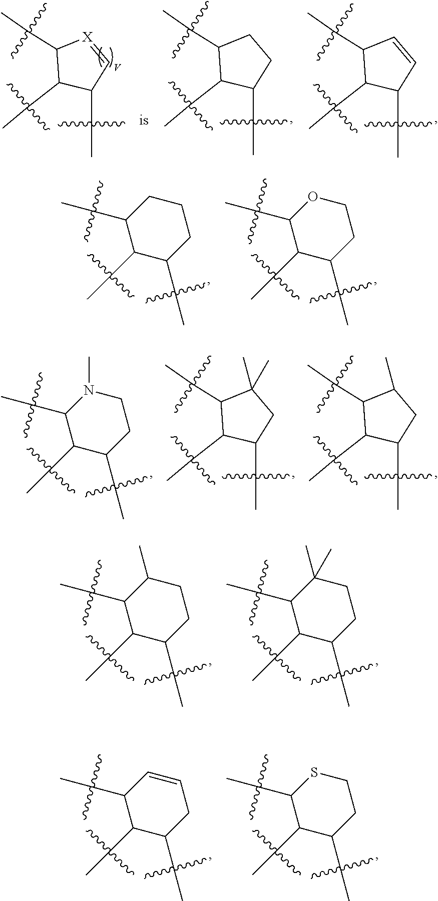

In a preferred embodiment, the compound of formula (I) may be compounds of formula (V-a)-(V-b), or a tautomer, cis- or trans-isomer, mesomer, racemate, enantiomer, diastereomer, or mixture thereof, or a pharmaceutically acceptable salt, solvate, or prodrug thereof,

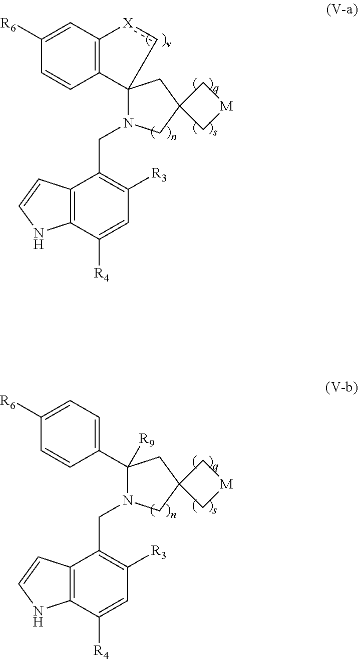

wherein,
M is O or CR_{f}R_{g};
R_{f} and R_{g} are independently selected from hydrogen, halogen and C₁₋₃ alkyl;
preferably, R_{f} and R_{g} are independently selected from hydrogen, fluorine;
q is 1, 2 or 3; and,
s is 0, 1 or 2.

In a preferred embodiment, the compound of formula (I) may be compounds of formula (VI-a)-(VI-l), or a tautomer, cis- or trans-isomer, mesomer, racemate, enantiomer, diastereomer, or mixture thereof, or a pharmaceutically acceptable salt, solvate, or prodrug thereof,

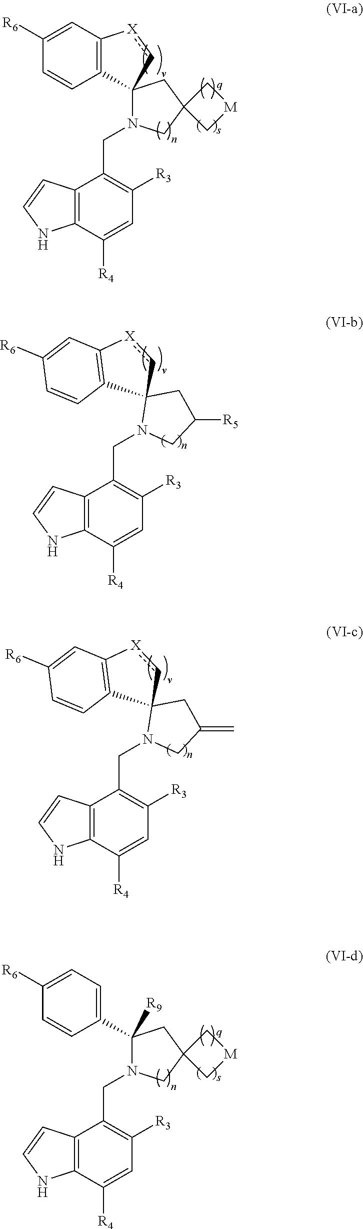

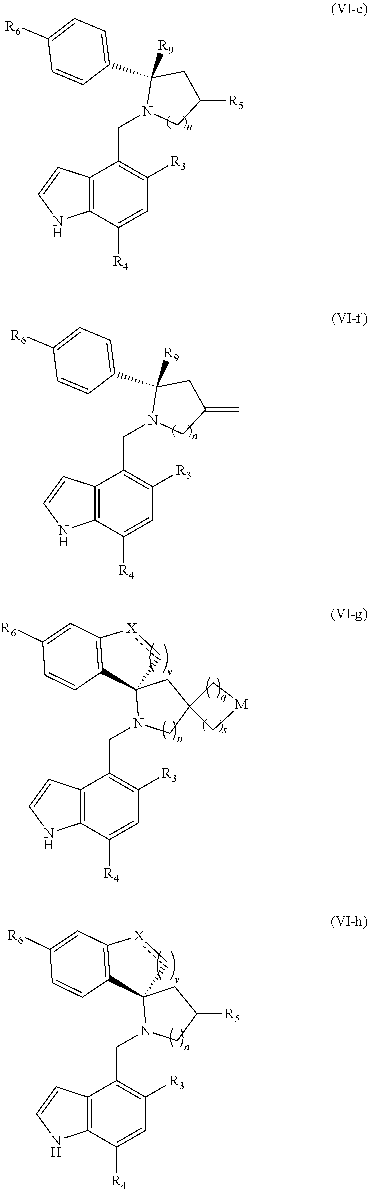

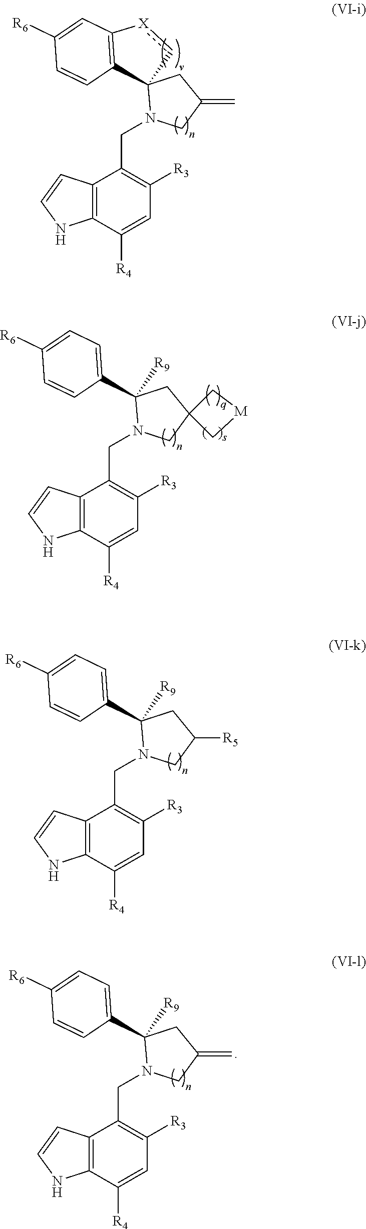

In a preferred embodiment,

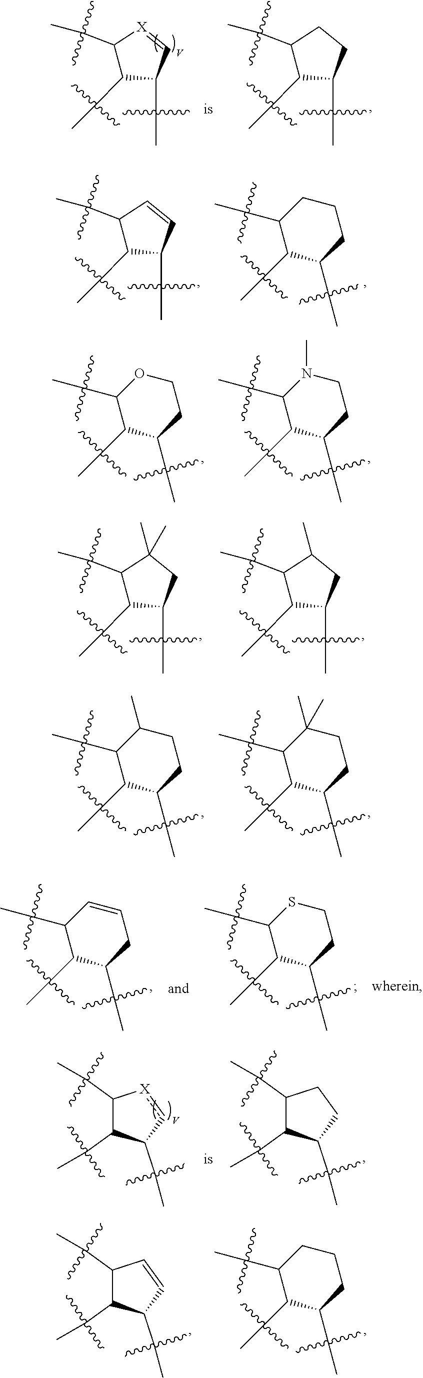

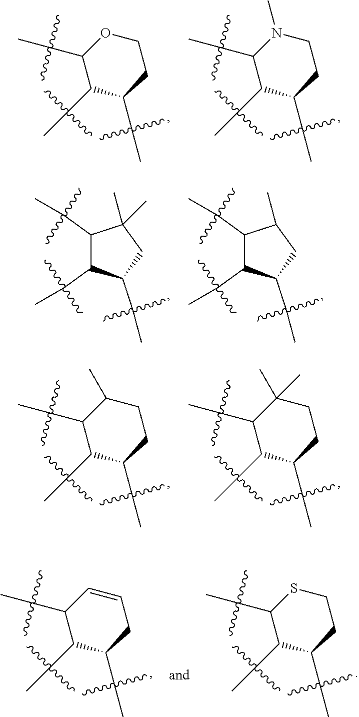

The present invention also provides a pharmaceutical composition, comprising a therapeutically effective amount of a compound of any formula (I)-(VII-f), or tautomer, or pharmaceutically acceptable salt thereof, together with one or more pharmaceutically acceptable carriers, diluents or excipients.

In some embodiments, in above stated pharmaceutical composition, the amount of the compound, tautomer, cis- or trans-isomer, mesomer, racemate, enantiomer, diastereomer, or mixture thereof, or pharmaceutically acceptable salts thereof is about 0.1-95% by weight of free base; preferably, is about 5-70%, e. g. 70%, 65%, 60%, 55%, 50%, 45%, 40%, 35%, 30%, 25%, 20%, 15%, 10%, 5%.

In some embodiment, above stated pharmaceutical composition is formulated as a tablet, capsule, liquid form or injection form.

In some embodiment, in above stated pharmaceutical composition, the amount of the compound, tautomer, cis- or trans-isomer, mesomer, racemate, enantiomer, diastereomer, or mixture thereof, or pharmaceutically acceptable salts thereof is about 1-1000 mg; preferably, is about 1-500 mg, more preferably, is about 1 mg, 2 mg, 3 mg, 5 mg, 10 mg, 20 mg, 40 mg, 50 mg, 60 mg, 80 mg, 100 mg, 200 mg, 300 mg, 400 m or 500 mg.

In some embodiment, the compound, tautomer, cis- or trans-isomer, mesomer, racemate, enantiomer, diastereomer, or mixture thereof, or pharmaceutically acceptable salts thereof is can be administered by any suitable route of administration, e.g. oral, parenteral, buccal, sublingual, nasal, rectal, intrathecal or transdermal administration, and the pharmaceutical compositions adapted accordingly.

In some embodiment, the compound, tautomer, cis- or trans-isomer, mesomer, racemate, enantiomer, diastereomer, or mixture thereof, or pharmaceutically acceptable salts is formulated as a solid or liquid form, e.g. syrups, suspension, emulsion, tablets, capsules, powders, granules or lozenges.

In another aspect, the present invention relates to a method of modulating complement alternative pathway activity, comprising administering to a subject in need thereof an effective amount of a compound any formula (I)-(VI-f), or a pharmaceutical composition comprising the same.

In another aspect, the present invention relates to a method of treating a disorder or a disease in a subject mediated by complement activation, in particular mediated by activation of the complement alternative pathway, comprising administering to a subject in need thereof an effective amount of a compound any formula (I)-(VI-f), or a pharmaceutical composition comprising the same.

In a preferred embodiment, the disease or disorder is selected from the group consisting of age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, ARDS, asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, glomerulonephritis and obesity.

### DETAILED DESCRIPTION OF THE INVENTION

Various publications, articles and patents are cited or described through the specification; each of these references is herein incorporated by references in its entirety. Discussion of documents, acts, materials, devices, articles or the like which has been included in the present specification is for the purpose of providing context for the disclosure. Such discussion is not an admission that any or all of these matters form part of the prior art with respect to the disclosure.

Given below are definitions of terms used in this application. Any term not defined herein takes the normal meaning as the skilled person would understand the term.

“Alkyl” refers to a saturated aliphatic hydrocarbon group including C1-C₂₀ straight chain and branched chain groups. Preferably an alkyl group is an alkyl having 1 to 12, sometimes preferably 1 to 6, sometimes more preferably 1 to 4, carbon atoms. Representative examples include, but are not limited to methyl, ethyl, n-propyl, isopropyl, n-butyl, isobutyl, tert-butyl, sec-butyl, n-pentyl, 1,1-dimethyl propyl, 1,2-dimethyl propyl, 2,2-dimethyl propyl, 1-ethyl propyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and the isomers of branched chain thereof. More preferably an alkyl group is a lower alkyl having 1 to 6 carbon atoms. Representative examples include, but are not limited to, methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, etc. The alkyl group can be substituted or unsubstituted. When substituted, the substituent group(s) can be substituted at any available connection point, preferably the substituent group(s) is one or more substituents independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group.

“Alkenyl” refers to an alkyl defined as above that has at least two carbon atoms and at least one carbon-carbon double bond, for example, vinyl, 1-propenyl, 2-propenyl, 1-, 2-, or 3-butenyl, etc., preferably C₂₋₂₀ alkenyl, more preferably C₂₋₁₂ alkenyl, and most preferably C₂₋₆ alkenyl. The alkenyl group can be substituted or unsubstituted. When substituted, the substituent group(s) is preferably one or more, sometimes preferably one to five, sometimes more preferably one to three, group(s) independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group.

“Alkynyl” refers to an alkyl defined as above that has at least two carbon atoms and at least one carbon-carbon triple bond, for example, ethynyl, 1-propynyl, 2-propynyl, 1-, 2-, or 3-butynyl etc., preferably C₂₋₂₀ alkynyl, more preferably C₂₋₁₂ alkynyl, and most preferably C₂₋₆ alkynyl. The alkynyl group can be substituted or unsubstituted. When substituted, the substituent group(s) is preferably one or more, sometimes preferably one to five, sometimes more preferably one to three, group(s) independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Alkylene” refers to a saturated linear or branched aliphatic hydrocarbon group, wherein having 2 residues derived by removing two hydrogen atoms from the same carbon atom of the parent alkane or two different carbon atoms. The straight or branched chain group containing 1 to 20 carbon atoms, preferably has 1 to 12 carbon atoms, more preferably 1 to 6 carbon atoms. Non-limiting examples of alkylene groups include, but are not limited to, methylene (—CH₂—), 1,1-ethylene (—CH (CH₃)—), 1,2-ethylene (—CH₂CH₂)—, 1,1-propylene (—CH (CH₂CH₃)—), 1,2-propylene (—CH₂CH (CH₃)—), 1,3-propylene (—CH₂CH₂CH₂—), 1,4-butylidene (—CH₂CH₂CH₂CH₂—) etc. The alkylene group can be substituted or unsubstituted. When substituted, the substituent group(s) is preferably one or more, sometimes preferably one to five, sometimes more preferably one to three, group(s) independently selected from the group consisting of selected from alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Alkenylene” refers to an alkylene defined as above that has at least two carbon atoms and at least one carbon-carbon double bond, preferably C₂₋₂₀ alkenylene, more preferably C₂₋₁₂ alkenylene, and most preferably C₂₋₆ alkenylene. Non-limiting examples of alkenylene groups include, but are not limited to, —CH═CH—, —CH═CHCH₂—, —CH═CHCH₂CH₂—, —CH₂CH═CHCH₂-etc. The alkenylene group can be substituted or unsubstituted. When substituted, the substituent group(s) is preferably one or more, sometimes preferably one to five, sometimes more preferably one to three, group(s) independently selected from the group consisting of selected from alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Alkynylene” refers to an alkynyl defined as above that has at least two carbon atoms and at least one carbon-carbon triple bond, preferably C₂₋₂₀ alkynylene, more preferably C₂₋₁₂ alkynylene, and most preferably C₂₋₆ alkynylene. Non-limiting examples of alkenylene groups include, but are not limited to, —CH═CH—, —CH═CHCH₂—, —CH═CHCH₂CH₂—, —CH₂CH═CHCH₂-etc. The alkynylene group can be substituted or unsubstituted. When substituted, the substituent group(s) is preferably one or more, sometimes preferably one to five, sometimes more preferably one to three, group(s) independently selected from the group consisting of selected from alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Cycloalkyl” refers to a saturated and/or partially unsaturated monocyclic or polycyclic hydrocarbon group having 3 to 20 carbon atoms, preferably 3 to 12 carbon atoms, more preferably 3 to 10 carbon atoms, and most preferably 3 to 8 carbon atoms or 3 to 6 carbon atoms.

Representative examples of monocyclic cycloalkyls include, but are not limited to, cyclopropyl, cyclobutyl, cyclopentyl, cyclopentenyl, cyclohexyl, cyclohexenyl, cyclohexadienyl, cycloheptyl, cycloheptatrienyl, cyclooctyl, etc. Polycyclic cycloalkyl includes a cycloalkyl having a spiro ring, fused ring or bridged ring.

“Spiro Cycloalkyl” refers to a 5 to 20 membered polycyclic group with rings connected through one common carbon atom (called a spiro atom), wherein one or more rings can contain one or more double bonds, but none of the rings has a completely conjugated pi-electron system. Preferably a spiro cycloalkyl is 6 to 14 membered, more preferably 7 to 10 membered, and most preferably 7 to 8 membered. According to the number of common spiro atoms, a spiro cycloalkyl is divided into mono-spiro cycloalkyl, di-spiro cycloalkyl, or poly-spiro cycloalkyl, and preferably refers to a mono-spiro cycloalkyl or di-spiro cycloalkyl, more preferably 4-membered/4-membered, 4-membered/5-membered, 4-membered/6-membered, 5-membered/5-membered, or 5-membered/6-membered mono-spiro cycloalkyl. Representative examples of spiro cycloalkyl include, but are not limited to the following substituents:

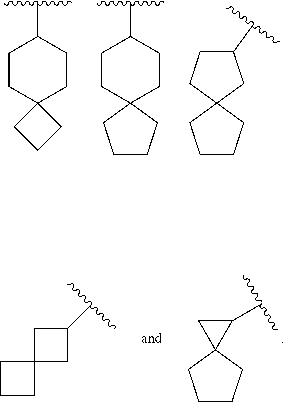

“Fused Cycloalkyl” refers to a 5 to 20 membered polycyclic hydrocarbon group, wherein each ring in the system shares an adjacent pair of carbon atoms with another ring, wherein one or more rings can contain one or more double bonds, but none of the rings has a completely conjugated pi-electron system. Preferably, a fused cycloalkyl group is 6 to 14 membered, more preferably 7 to 10 membered, and most preferably 7 to 8 membered. According to the number of membered rings, fused cycloalkyl is divided into bicyclic, tricyclic, tetracyclic or polycyclic fused cycloalkyl, and preferably refers to a bicyclic or tricyclic fused cycloalkyl, more preferably 5-membered/5-membered, or 5-membered/6-membered bicyclic fused cycloalkyl. Representative examples of fused cycloalkyls include, but are not limited to, the following substituents:

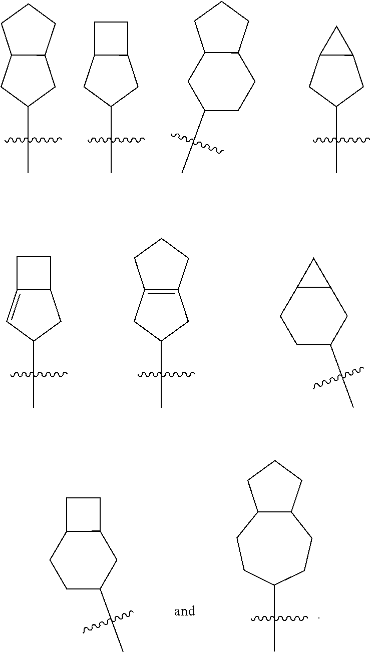

“Bridged Cycloalkyl” refers to a 5 to 20 membered polycyclic hydrocarbon group, wherein every two rings in the system share two disconnected carbon atoms. The rings can have one or more double bonds, but have no completely conjugated pi-electron system. Preferably, a bridged cycloalkyl is 6 to 14 membered, more preferably 7 to 10 membered, and most preferably 7 to 8 membered. According to the number of membered rings, bridged cycloalkyl is divided into bicyclic, tricyclic, tetracyclic or polycyclic bridged cycloalkyl, and preferably refers to a bicyclic, tricyclic or tetracyclic bridged cycloalkyl, more preferably a bicyclic or tricyclic bridged cycloalkyl. Representative examples of bridged cycloalkyls include, but are not limited to, the following substituents:

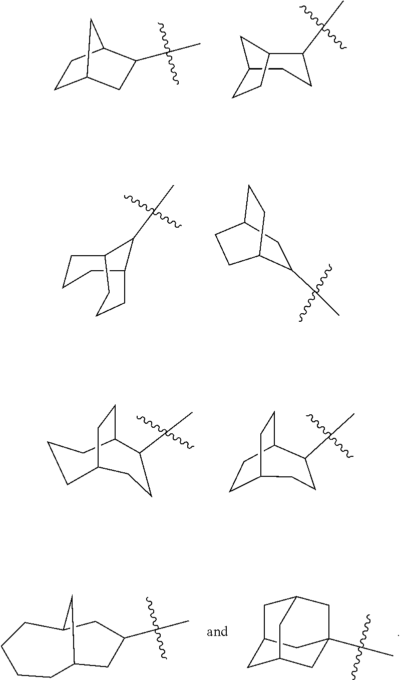

The cycloalkyl can be fused to the ring of an aryl, heteroaryl or heterocyclic alkyl, wherein the ring bound to the parent structure is cycloalkyl. Representative examples include, but are not limited to indanylacetic, tetrahydronaphthalene, benzocycloheptyl and so on. The cycloalkyl is optionally substituted or unsubstituted. When substituted, the substituent group(s) is preferably one or more, sometimes preferably one to five, sometimes more preferably one to three, substituents independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group. Representative examples include, but are not limited to, the following substituents:

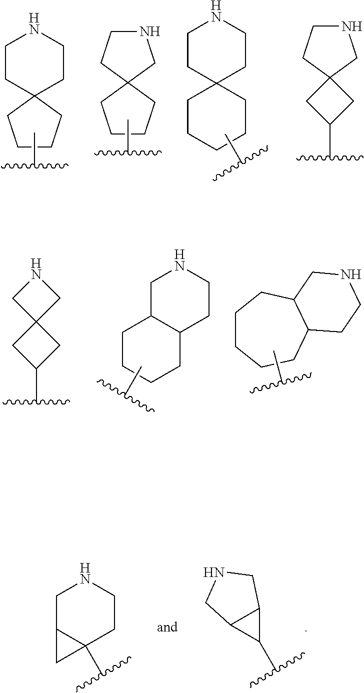

“Heterocyclyl” refers to a 3 to 20 membered saturated and/or partially unsaturated monocyclic or polycyclic hydrocarbon group having one or more, sometimes preferably one to five, sometimes more preferably one to three, heteroatoms selected from the group consisting of N, O, and S(O)ₘ (wherein m is 0, 1, or 2) as ring atoms, but excluding —O—O—, —O—S— or —S— S— in the ring, the remaining ring atoms being C. Preferably, heterocyclyl is a 3 to 12 membered having 1 to 4 heteroatoms; more preferably a 3 to 10 membered having 1 to 3 heteroatoms; most preferably a 5 to 6 membered having 1 to 2 heteroatoms. Representative examples of monocyclic heterocyclyls include, but are not limited to, pyrrolidyl, piperidyl, piperazinyl, morpholinyl, sulfo-morpholinyl, homopiperazinyl, and so on. Polycyclic heterocyclyl includes the heterocyclyl having a spiro ring, fused ring or bridged ring.

“Spiro heterocyclyl” refers to a 5 to 20 membered polycyclic heterocyclyl with rings connected through one common carbon atom (called a spiro atom), wherein said rings have one or more, sometimes preferably one to five, sometimes more preferably one to three, heteroatoms selected from the group consisting of N, O, and S(O)ₘ (wherein m is 0.1 or 2) as ring atoms, the remaining ring atoms being C, wherein one or more rings can contain one or more double bonds, but none of the rings has a completely conjugated pi-electron system. Preferably a spiro heterocyclyl is 6 to 14 membered, more preferably 7 to 10 membered, and most preferably 7 to 8 membered. According to the number of common spiro atoms, spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, more preferably 4-membered/4-membered, 4-membered/5-membered, 4-membered/6-membered, 5-membered/5-membered, or 5-membered/6-membered mono-spiro heterocyclyl. Representative examples of spiro heterocyclyl include, but are not limited to the following substituents:

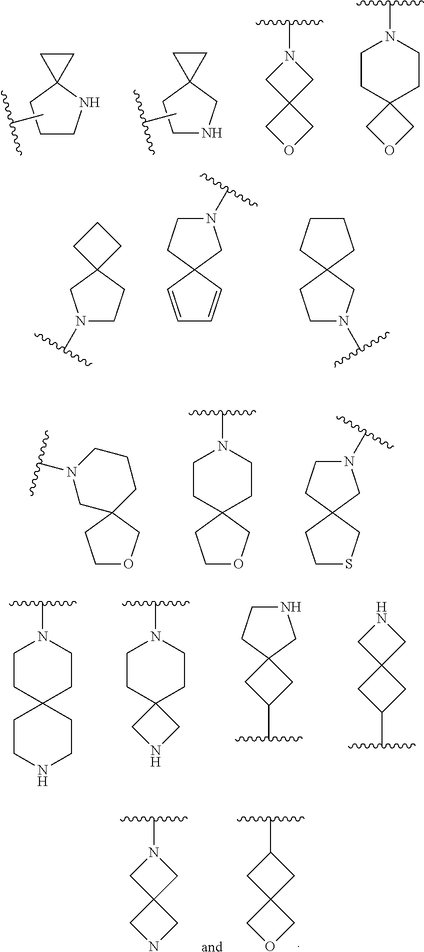

“Fused Heterocyclyl” refers to a 5 to 20 membered polycyclic heterocyclyl group, wherein each ring in the system shares an adjacent pair of carbon atoms with the other ring, wherein one or more rings can contain one or more double bonds, but none of the rings has a completely conjugated pi-electron system, and wherein said rings have one or more, sometimes preferably one to five, sometimes more preferably one to three, heteroatoms selected from the group consisting of N, O, and S(O)ₚ (wherein p is 0, 1, or 2) as ring atoms, the remaining ring atoms being C. Preferably a fused heterocyclyl is 6 to 14 membered, more preferably 7 to 10 membered, and most preferably 7 to 8 membered. According to the number of membered rings, fused heterocyclyl is divided into bicyclic, tricyclic, tetracyclic or polycyclic fused heterocyclyl, preferably refers to bicyclic or tricyclic fused heterocyclyl, more preferably 5-membered/5-membered, or 5-membered/6-membered bicyclic fused heterocyclyl. Representative examples of fused heterocyclyl include, but are not limited to, the following substituents:

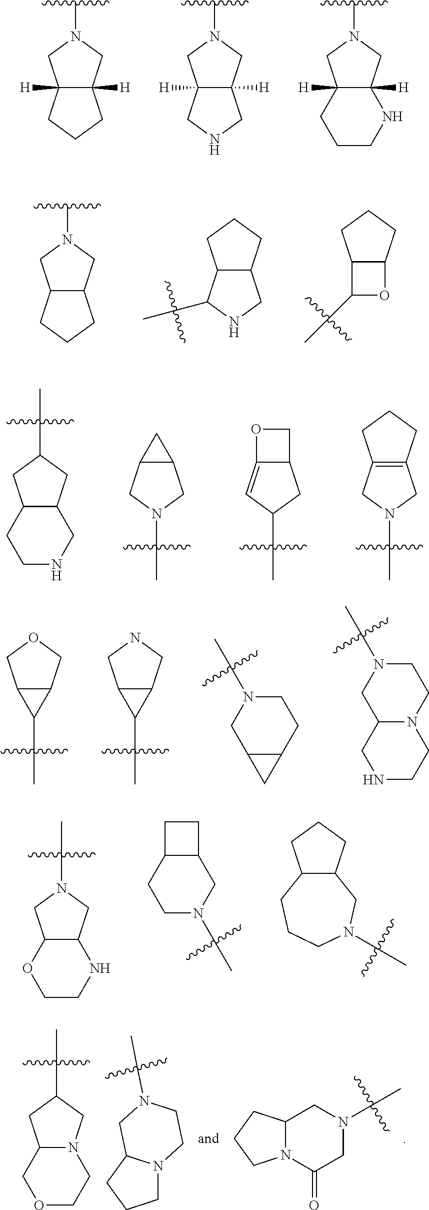

“Bridged Heterocyclyl” refers to a 5 to 14 membered polycyclic heterocyclic alkyl group, wherein every two rings in the system share two disconnected atoms, the rings can have one or more double bonds, but have no completely conjugated pi-electron system, and the rings have one or more heteroatoms selected from the group consisting of N, O, and S(O)ₘ (wherein m is 0, 1, or 2) as ring atoms, the remaining ring atoms being C. Preferably a bridged heterocyclyl is 6 to 14 membered, more preferably 7 to 10 membered, and most preferably 7 to 8 membered. According to the number of membered rings, bridged heterocyclyl is divided into bicyclic, tricyclic, tetracyclic or polycyclic bridged heterocyclyl, and preferably refers to bicyclic, tricyclic or tetracyclic bridged heterocyclyl, more preferably bicyclic or tricyclic bridged heterocyclyl. Representative examples of bridged heterocyclyl include, but are not limited to, the following substituents:

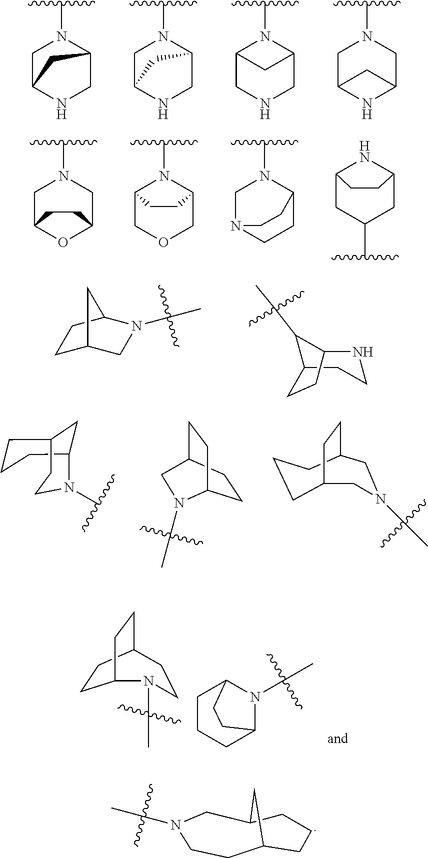

The ring of said heterocyclyl can be fused to the ring of an aryl, heteroaryl or cycloalkyl, wherein the ring bound to the parent structure is heterocyclyl. Representative examples include, but are not limited to the following substituents:

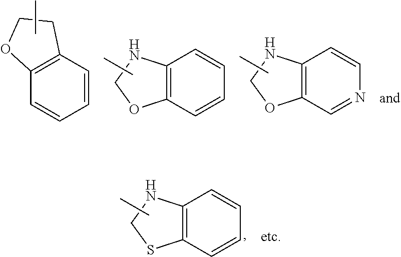

The heterocyclyl is optionally substituted or unsubstituted. When substituted, the substituent group(s) is preferably one or more, sometimes preferably one to five, sometimes more preferably one to three, group(s) independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio, heterocylic and alkylthio.

“Aryl” refers to a 6 to 14 membered all-carbon monocyclic ring or a polycyclic fused ring (a “fused” ring system means that each ring in the system shares an adjacent pair of carbon atoms with another ring in the system) group, and has a completely conjugated pi-electron system. Preferably aryl is 6 to 10 membered, such as phenyl and naphthyl, most preferably phenyl. The aryl can be fused to the ring of heteroaryl, heterocyclyl or cycloalkyl, wherein the ring bound to parent structure is aryl. Representative examples include, but are not limited to, the following substituents:

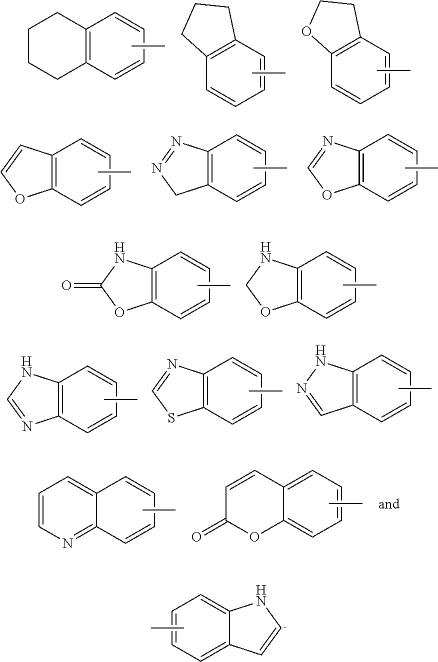

The aryl group can be substituted or unsubstituted. When substituted, the substituent group(s) is preferably one or more, sometimes preferably one to five, sometimes more preferably one to three, substituents independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio, heterocylic and alkylthio.

“Heteroaryl” refers to an aryl system having 1 to 4 heteroatoms selected from the group consisting of O, S and N as ring atoms and having 5 to 14 annular atoms. Preferably a heteroaryl is 5- to 10-membered, more preferably 5- or 6-membered, for example, thiadiazolyl, pyrazolyl, oxazolyl, oxadiazolyl, imidazolyl, triazolyl, thiazolyl, furyl, thienyl, pyridyl, pyrrolyl, N-alkyl pyrrolyl, pyrimidinyl, pyrazinyl, imidazolyl, tetrazolyl, isoxazolyl and the like. The heteroaryl can be fused with the ring of an aryl, heterocyclyl or cycloalkyl, wherein the ring bound to parent structure is heteroaryl. Representative examples include, but are not limited to, the following substituents:

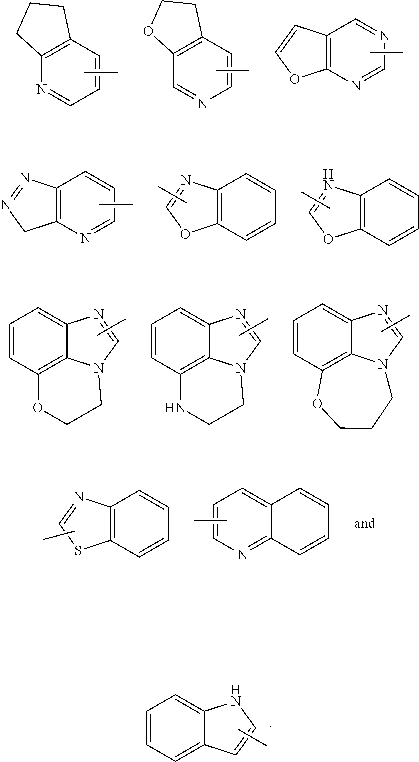

The heteroaryl group can be substituted or unsubstituted. When substituted, the substituent group(s) is preferably one or more, sometimes preferably one to five, sometimes more preferably one to three, substituents independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio, heterocylic alkylthio.

“Alkoxy” refers to both an —O-(alkyl) and an —O-(unsubstituted cycloalkyl) group, wherein the alkyl is defined as above. Representative examples include, but are not limited to, methoxy, ethoxy, propoxy, butoxy, cyclopropyloxy, cyclobutyloxy, cyclopentyloxy, cyclohexyloxy, and the like. The alkoxyl can be substituted or unsubstituted. When substituted, the substituent is preferably one or more, sometimes preferably one to five, sometimes more preferably one to three, substituents independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Haloalkoxy” refers to an alkoxy group substituted by one or more halogen(s), wherein the alkoxy is as defined above.

The hydrogen atom of the present invention can be substituted by its isotope deuterium. Any of the hydrogen atoms in the compounds of the examples of the present invention can also be substituted by deuterium atom.

“Bond” refers to a covalent bond using a sign of “—”.

“Hydroxyalkyl” refers to an alkyl group substituted by a hydroxy group, wherein alkyl is as defined above.

“Hydroxyl” or “hydroxy” refers to an —OH group.

“Halogen” or “halo” refers to fluoro, chloro, bromo or iodo atoms.

“Amino” refers to a —NH₂ group.

“Cyano” refers to a —CN group.

“Nitro” refers to a —NO₂ group.

“Oxo group” refers to a ═O group.

“Carboxyl” refers to a —C(O) OH group.

“Alkoxycarbonyl” refers to a —C(O)O(alkyl) or (cycloalkyl) group, wherein the alkyl and cycloalkyl are defined as above.

Where it is stated that groups or substituents are “independently selected from” (and variants thereof) a list of choices, it is meant that the choice for any one of such groups or substituents does not determine the choice for any other one of such groups or substituents. By way of an illustration, but not as a limitation, the term “A and B are independently selected from a and b” or “each of A and B is independently selected from a and b” is meant to encompass selections where A is a and B is a, A is b and B is b, A is a and B is b, and A is b and B is a.

“Optional” or “optionally” means that the event or circumstance described subsequently can, but need not, occur, and the description includes the instances in which the event or circumstance may or may not occur. For example, “the heterocyclic group optionally substituted by an alkyl” means that an alkyl group can be, but need not be, present, and the description includes the case of the heterocyclic group being substituted with an alkyl and the heterocyclic group being not substituted with an alkyl.

“Substituted” refers to one or more hydrogen a members in a group independently substituted with a corresponding number of substituents. In some embodiments, the number of such hydrogen members is up to 5. In other embodiments it is between 1 and 3. It goes without saying that the substituents exist in their only possible chemical position. The person skilled in the art is able to determine if the substitution is possible or impossible without paying excessive efforts by experiment or theory. For example, the combination of amino or hydroxyl group having free hydrogen and carbon atoms having unsaturated bonds (such as olefinic) may be unstable.

A “pharmaceutical composition” refers to a mixture of one or more of the compounds described in the present invention or physiologically/pharmaceutically acceptable salts or prodrugs thereof and other chemical components such as physiologically/pharmaceutically acceptable carriers and excipients. The purpose of a pharmaceutical composition is to facilitate administration of a compound to an organism, which is conducive to the absorption of the active ingredient and thus displaying biological activity.

“Pharmaceutically acceptable salts” refer to salts of the compounds of the invention, such salts being safe and effective when used in a mammal and have corresponding biological activity.

### EXAMPLES

The following examples serve to illustrate the invention, but the examples should not be considered as limiting the scope of the invention. If specific conditions for the experimental method are not specified in the examples of the present invention, they are generally in accordance with conventional conditions or recommended conditions of the raw materials and the product manufacturer. The reagents without a specific source indicated are commercially available, conventional reagents.

The structure of each compound is identified by nuclear magnetic resonance (NMR) and/or mass spectrometry (MS). NMR chemical shifts (δ) are given in 10⁻⁶ (ppm). NMR is determined by Varian Mercury 300 MHz Bruker Avance III 400 MHz machine. The solvents used are deuterated-dimethyl sulfoxide (DMSO-d₆), deuterated-chloroform (CDCl₃) and deuterated-methanol (CD₃OD).

High performance liquid chromatography (HPLC) is determined on an Agilent 1200DAD high pressure liquid chromatography spectrometer (Sunfire C18 150×4.6 mm chromatographic column) and a Waters 2695-2996 high pressure liquid chromatography spectrometer (Gimini C18 150×4.6 mm chromatographic column). Liquid Chromatography Mass Spectrometry (LCMS) is determined on an Agilent 1200 high pressure liquid chromatography spectrometer & mass spectrometry (Sunfire C18 4.6*50 mm 3.5 μm chromatographic column) and an Agilent 19091S-433 HP-5 high pressure liquid chromatography spectrometer & mass spectrometry (XBridge C18 4.6*50 mm 3.5 μm chromatographic column).

Chiral High performance liquid chromatography (HPLC) is determined on SFC Thar 80 & 150 & 200 (waters.)

The average rates of ATPase inhibition, and the IC₅₀ values are determined by Victor Nivo multimode plate reader (PerkinElmer, USA).

The thin-layer silica gel plates used in thin-layer chromatography are Yantai Xinnuo silica gel plate. The dimension of the plates used in TLC is 0.15 mm to 0.2 mm, and the dimension of the plates used in thin-layer chromatography for product purification was 0.4 mm to 0.5 mm.

Column chromatography generally uses Qingdao Haiyang 200 to 300 mesh silica gel as carrier.

The known starting material of the invention can be prepared by the conventional synthesis method in the prior art, or can be purchased from ABCR GmbH & Co. KG, Acros Organics, Aldrich Chemical Company, Accela ChemBio Inc or Dari chemical Company, etc.

Unless otherwise stated in the examples, the following reactions are placed under argon atmosphere or nitrogen atmosphere.

The term “argon atmosphere” or “nitrogen atmosphere” means that a reaction flask is equipped with a balloon having 1 L of argon or nitrogen.

The term “hydrogen atmosphere” means that a reaction flask was equipped with a balloon having 1 L of hydrogen.

MS is mass spectroscopy with (+) referring to the positive mode which generally gives a M+1 (or M+H) absorption where M=the molecular mass.

### Synthetic Procedures:

Synthetic procedure of tert-butyl 4-(chloromethyl)-5-methoxy-7-methyl-1H-indole-1-carboxylate, Int A

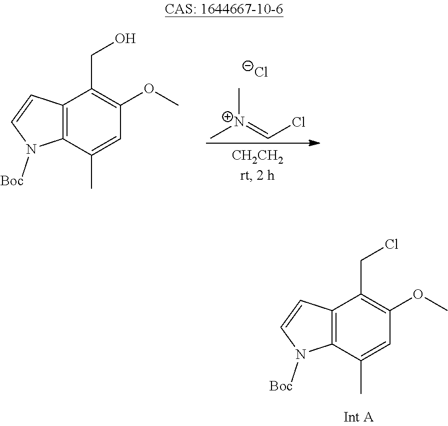

To a solution of tert-butyl 4-(hydroxymethyl)-5-methoxy-7-methyl-1H-indole-1-carboxylate (CAS: 1644667-10-6-950 mg, 3.45 mmol) in CH₂Cl₂ (10 mL) was added (chloromethylene)dimethyliminium chloride (711 mg, 5.56 mmol) in one portion at room temperature under nitrogen and the mixture stirred at that temperature for 2 h. The reaction mixture was cooled to 0 °C, then quenched with 5% aq. NaHCO₃. The mixture was extracted with CH₂Cl₂ (3×20 mL). The combined organic layers were washed with brine (20 mL), dried over anhydrous Na₂SO₄, filtered, and concentrated. The crude product, tert-butyl 4-(chloromethyl)-5-methoxy-7-methyl-1H-indole-1-carboxylate, Int A (900 mg), was obtained as a yellow oil. LCMS (m/z): [M-Cl]⁺ calc'd for C₁₆H₂₀NO₃, 274.1; found, 274.1.

### Synthetic procedure of 3,3-difluoro-1′-((5-methoxy-7-methyl-1H-indol-4-yl)methyl)-2″,3″-dihydrodispiro[cyclobutane-1,4′-piperidine-2′,1″-indene]-5″-carboxylic acid

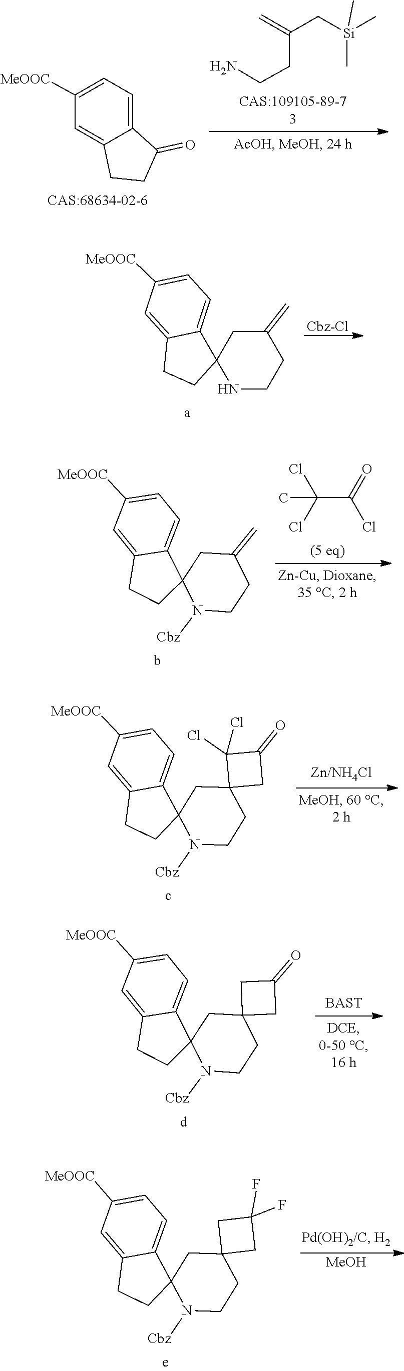

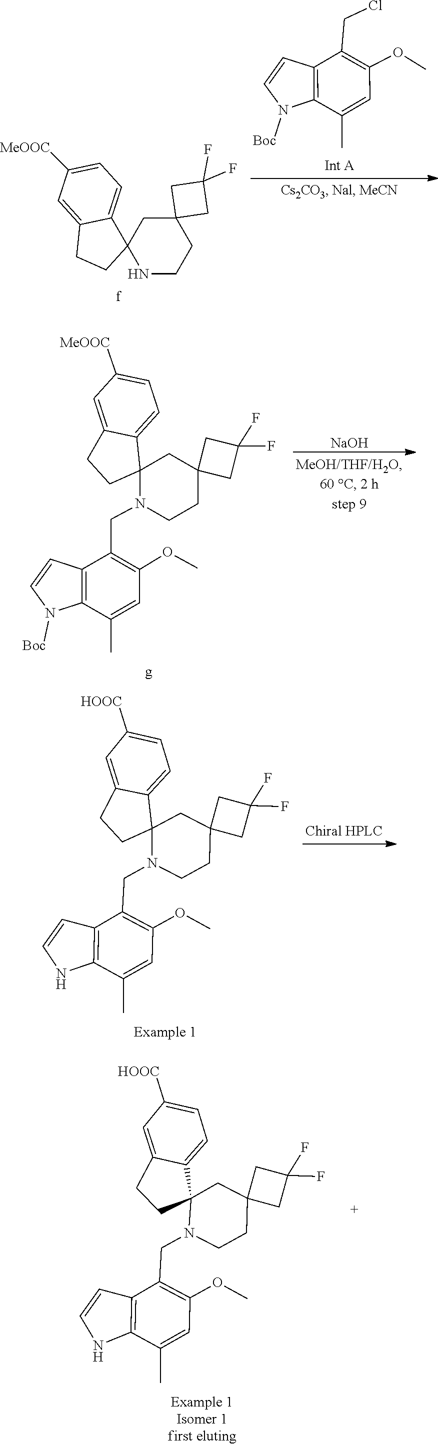 [SMILES: COC(=O)c1ccc2c(c1)CCC21CC2(CCN1)CC(F)(F)C2 ; COc1cc(C)c2c(ccn2C(=O)OC(C)(C)C)c1CCl ; COC(=O)c1ccc2c(c1)CCC21CC2(CCN1Cc1c(OC)cc(C)c3c1ccn3C(=O)OC(C)(C)C)CC(F)(F)C2 ; "NaOH" ; COc1cc(C)c2[nH]ccc2c1CN1CCC2(CC(F)(F)C2)CC12CCc1cc(C(=O)O)ccc12 ; COc1cc(C)c2[nH]ccc2c1CN1CCC2(CC(F)(F)C2)C[C@]12CCc1cc(C(=O)O)ccc12]

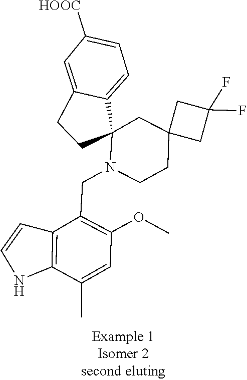 [SMILES: COc1cc(C)c2[nH]ccc2c1CN1CCC2(CC(F)(F)C2)C[C@]12CCc1cc(C(=O)O)ccc12]

### Step 1

A solution of methyl 1-oxo-2,3-dihydro-1H-indene-5-carboxylate (750 mg, 3.94 mmol), 3-((trimethylsilyl)-methyl) but-3-en-1-amine (1.12 g, 7.10 mmol) and AcOH (355 mg, 5.91 mmol) in MeOH was stirred at 50 °C for 40 h. The reaction mixture was neutralized with saturated NaHCO₃ solution and then extracted with DCM (50 mL×3). The combined organic layers were washed with brine (50 mL), dried over with anhydrous Na₂SO₄, filtered and concentrated. The residue was purified by flash column chromatography on silica gel (PE/EtOAc=1/1) to afford compound a (860 mg, 85%) as a black oil. MS: m/z=258.1 (M+1, ESI+).

### Step 2

To a solution of methyl 4′-methylene-2,3-dihydrospiro[indene-1,2′-piperidine]-5-carboxylate (820 mg, 3.19 mmol) and K₂CO₃ (1.32 g, 9.56 mmol) in THF (10 mL) stirred under nitrogen at 0 °C was added Cbz-Cl (815 mg, 4.78 mmol) dropwise. The reaction mixture was stirred at 25 °C for 16 h. The reaction mixture was quenched with NaHCO₃ solution (30 mL) and then extracted with EtOAc (30 mL×3). The combined organic layers were washed with brine (30 mL), dried over with anhydrous Na₂SO₄, filtered and concentrated. The residue was purified by flash column chromatography on silica gel (PE/EtOAc=5/1) to afford 1′-benzyl 5-methyl 4′-methylene-2,3-dihydrospiro[indene-1,2′-piperidine]-1′,5-dicarboxylate, b (1.05 g, 84%) as a colorless oil. MS: m/z=391.9 (M+1, ESI+).

### Step 3

To a suspension of Cu—Zn (3.5 g, 3% Cu) and methyl 4′-methylene-2,3-dihydrospiro[indene-1,2′-piperidine]-1′,5-dicarboxylate, b (1.0 g, 2.4 mmol, 1.0 equiv) in dioxane (20 mL, 20 V) was added trichloroacetyl chloride (4.4 g, 24 mmol, 10 equiv) at 20 °C over 30 min under a nitrogen atmosphere. The mixture was heated to 35 °C for 3 h. The reaction mixture was quenched by NH₄Cl solution. The solid was filtered off and the filtrate was extracted with ethyl acetate (30 mL×3, 30 V), washed with brine (40 mL, 40 V). The combined filtrate was dried over Na₂SO₄, concentrated under vacuum to give the oil crude product 1′-benzyl 5″-methyl 2,2-dichloro-3-oxo-2″,3″-dihydrodispiro[cyclobutane-1,4′-piperidine-2′,1″-indene]-1′,5″-dicarboxylate, c (1.2 g crude) which was used in the next step without further purification. LCMS (m/z): [M+H]⁺ calc'd for C₂₆H₂₆Cl₂NO₅, 502.1; found, 502.3.

### Step 4

To a solution of 1′-benzyl 5″-methyl 2,2-dichloro-3-oxo-2″,3″-dihydrodispiro[cyclobutane-1,4′-piperidine-2′,1″-indene]-1′,5″-dicarboxylate, c (1.2 g, crude) in MeOH (50 mL) was added NH₄Cl (2.6 g, 48 mmol) and Zinc (1.6 g, 24 mmol) at rt. The reaction mixture was stirred at 60 °C for 2 h, and then filtered. The filtrate was concentrated in vacuum and the residue was purified by flash column chromatography (ethyl acetate:petroleum ether=2/1) on silica gel to afford product 1′-benzyl 5″-methyl 3-oxo-2″,3″-dihydrodispiro[cyclobutane-1,4′-piperidine-2′,1″-indene]-1′,5″-dicarboxylate, d as an oil (500 mg, two step yield 50%). LCMS (m/z): [M+H]⁺ calc'd for C₂₆H₂₈NO₅, 434.2; found, 434.0.

### Step 5

1′-benzyl 5″-methyl 3-oxo-2″,3″-dihydrodispiro[cyclobutane-1,4′-piperidine-2′,1″-indene]-1′,5″-dicarboxylate, d (500 mg, 1.15 mmol) was dissolved in BAST (1.5 mL) at 0 °C. The reaction mixture was stirred at 50 °C for 12 h. The reaction was cooled to room temperature and 15 mL ethyl acetate was added. The reaction mixture was poured into ice (20 mL) very carefully. The residue was extracted with dichloromethane (30 mL×3), washed with brine (30 mL), dried over Na₂SO₄. The filtrate was concentrated in vacuum and purified by flash column chromatography (ethyl acetate:petroleum ether=1/5) on silica gel to afford product 1′-benzyl 5″-methyl 3,3-difluoro-2″,3″-dihydrodispiro[cyclobutane-1,4′-piperidine-2′,1″-indene]-1′,5″-dicarboxylate, e as an oil (300 mg, yield 57%). LCMS (m/z): [M+H]⁺ calc'd for C₂₆H₂₈F₂NO₄, 456.2; found, 456.0.

### Step 6

To a solution of 1′-benzyl 5″-methyl 3,3-difluoro-2″,3″-dihydrodispiro[cyclobutane-1,4′-piperidine-2′,1″-indene]-1′,5″-dicarboxylate, e (240 mg, 0.527 mmol) in MeOH (5 mL) stirred at 25 °C was added Pd (OH) 2/C (100 mg). The reaction mixture was stirred at 25 °C under H₂ for 2 h. After filtration, the solution was concentrated under vacuum to afford methyl 3,3-difluoro-2″,3″-dihydrodispiro[cyclobutane-1,4′-piperidine-2′,1″-indene]-5″-carboxylate, f as an oil (155 mg, yield 92%). LCMS (m/z): [M+H]⁺ calc'd for C₁₈H₂₂F₂NO₂, 322.1; found, 322.1.

### Step 7

A solution of methyl 3,3-difluoro-2″,3″-dihydrodispiro[cyclobutane-1,4′-piperidine-2′,1″-indene]-5″-carboxylate, f (155 mg, 0.48 mmol), tert-butyl 4-(chloromethyl)-5-methoxy-7-methyl-1H-indole-1-carboxylate (180 mg, 0.58 mmol), Cs₂CO₃ (471 mg, 1.45 mmol) and NaI (109 mg, 0.72 mmol) in acetonitrile (3 mL) was stirred at 80 °C for 2 h. After filtration, the solution was concentrated under vacuum, and the residue was purified by flash column chromatography on silica gel (PE/EtOAc=10/1) to afford methyl 1′-((1-(tert-butoxycarbonyl)-5-methoxy-7-methyl-1H-indol-4-yl)methyl)-3,3-difluoro-2″,3″-dihydrodispiro[cyclobutane-1,4′-piperidine-2′,1″-indene]-5″-carboxylate, g (180 mg, 63%) as white solid. LCMS (m/z): [M+H]⁺ calc'd for C₃₄H₄₁F₂N₂O₅, 595.1; found, 595.1.

### Step 8

A solution of methyl 1′-((1-(tert-butoxycarbonyl)-5-methoxy-7-methyl-1H-indol-4-yl)methyl)-3,3-difluoro-2″,3″-dihydrodispiro[cyclobutane-1,4′-piperidine-2′,1″-indene]-5″-carboxylate (180 mg, 0.30 mmol) and NaOH (60 mg, 1.51 mmol) in MeOH/THF/H₂O (1/1/0.2 mL) was stirred at 60 °C for 1 h. The reaction mixture was neutralized with saturated citric acid solution and then extracted with DCM/MeOH (10/1, 20 mL×3). The combined organic layers were concentrated under vacuum and the residue was purified by pre-HPLC (Acetonitrile-H2O, 0.1% FA) and SFC (Column: CHIRALPAK AD-H 250 mm×20 mm, 5 μm; Mobile phase: 40% EtOH [0.2% NH₄OH] in CO₂; Flow rate: 12.5 mL/min; Column temperature: 38 °C, Retention time=3.54 min) to afford Example 1 Isomer 1 (36.27 mg, 25%) and (Column: CHIRALPAK AD-H 250 mm×20 mm, 5 μm; Mobile phase: 40% EtOH [0.2% NH₄OH] in CO₂; Flow rate: 12.5 mL/min; Column temperature: 38 °C, Retention time=4.75 min) Example 1 isomer 2 (42.33 mg, 29%, white solid) as a formic acid salt. MS: m/z=481.0 (M+1, ESI+).
Example 1 isomer 1: ¹HNMR (400 MHz, CD₃OD) δ 8.08 (m, 1H), 7.98 (s, 1H), 7.60 (d, J=8.1 Hz, 1H), 7.28 (d, J=3.1 Hz, 1H), 6.70 (s, 1H), 6.23 (d, J=3.0 Hz, 1H), 4.15-4.04 (m, 1H), 4.02-3.89 (m, 1H), 3.65 (s, 3H), 3.52-3.41 (m, 2H), 3.27-3.11 (m, 2H), 2.95-2.75 (m, 2H), 2.73-2.60 (m, 1H), 2.56-2.42 (m, 6H), 2.40-2.29 (m, 1H), 2.11-1.94 (m, 3H).
Example 1 isomer 2: ¹HNMR (400 MHz, CD₃OD) δ 8.44 (s, 1H), 8.14-8.08 (m, 1H), 8.01 (s, 1H), 7.63 (d, J=8.1 Hz, 1H), 7.29 (d, J=3.2 Hz, 1H), 6.71 (s, 1H), 6.24 (d, J=3.1 Hz, 1H), 4.17-4.08 (m, 1H), 4.06-3.95 (m, 1H), 3.64 (s, 3H), 3.56-3.47 (m, 2H), 3.28-3.14 (m, 2H), 2.98-2.77 (m, 2H), 2.75-2.64 (m, 1H), 2.58-2.44 (m, 6H), 2.43-2.29 (m, 1H), 2.14-1.96 (m, 3H).

The following examples were synthesized using the ester hydrolysis procedure described above using appropriate starting materials:

TABLE A

| | | Isomer Separation | | | |
|---|---|---|---|---|---|
| | | Method and | | | |
| | | Retention Time (if | | | |
| Name and Stucture | Ex. # | any) | LCMS + ¹H NMR | | |
| 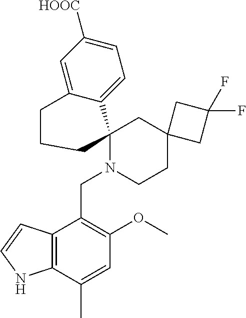 [SMILES: COc1cc(C)c2[nH]ccc2c1CN1CCC2(CC(F)(F)C2)C[C@]12CCCc1cc(C(=O)O)ccc12] | 2 (Isomer 1) | Column: CHIRALPAK AD-H 250 mm × 20 mm, 5 μm; Mobile phase: 40% EtOH [0.2% NH4OH] in CO₂ Flow rate: 12.5 mL/min Column temperature: 37.5 °C Retention time = 3.61 min | LC-MS: m/z = 495.1. (M + 1, ESI+). ¹H NMR (400 MHz, CD3OD) δ 8.46 (s, 0.28H), 7.97 (d, J = 7.4 Hz, 1H), 7.82 (s, 1H), 7.76 (d, J = 8.3 Hz, 1H), 7.29 (d, J = 3.1 Hz, 1H), 6.74 (s, 1H), 6.37 (d, J = 3.1 Hz, 1H), 4.22 (d, J = 12.2 Hz, 1H), 4.07 (d, J = 12.1 Hz, 1H), 3.73 (s, 3H), 3.54 (s, 1H), 3.41 (d, J = 12.4 Hz, 1H), 3.05-2.85 (m, 2H), 2.79-2.62 (m, 2H), 2.48 (s, 3H), 2.45-2.19 (m, 5H), 2.15-2.07 (m, 2H), 2.03-1.93 (m, 3H). | | |
| 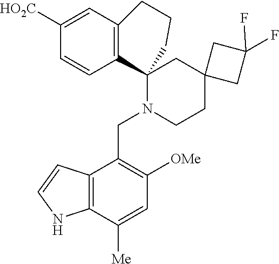 [SMILES: COc1cc(C)c2[nH]ccc2c1CN1CCC2(CC(F)(F)C2)C[C@@]12CCCc1cc(C(=O)O)ccc12] | 2 (Isomer 2) | Column: CHIRALPAK AD-H 250 mm × 20 mm, 5 μm; Mobile phase: 40% EtOH [0.2% NH4OH] in CO₂ Flow rate: 12.5 mL/min Column temperature: 37.5 °C Retention time = 4.47 min | LC-MS: m/z = 495.1. (M + 1, ESI+). ¹H NMR (400 MHz, CD3OD) δ 7.98 (d, J = 8.2 Hz, 1H), 7.82 (s, 1H), 7.76 (d, J = 8.3 Hz, 1H), 7.29 (d, J = 3.2 Hz, 1H), 6.74 (s, 1H), 6.37 (d, J = 3.2 Hz, 1H), 4.21 (d, J = 12.7 Hz, 1H), 4.07 (d, J = 12.2 Hz, 1H), 3.73 (s, 3H), 3.57 (s, 1H), 3.41 (d, J = 10.3 Hz, 1H), 3.14-2.83 (m, 2H), 2.81- 2.58 (m, 2H), 2.49 (s, 3H), 2.45- 2.18 (m, 5H), 2.17-2.06 (m,, 2H), 2.03-1.91 (m, 3H). | | |
| 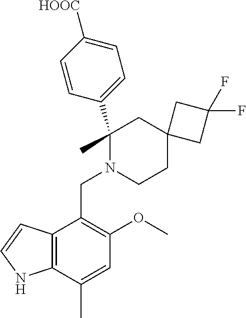 [SMILES: COc1cc(C)c2[nH]ccc2c1CN1CCC2(CC(F)(F)C2)C[C@@]1(C)c1ccc(C(=O)O)cc1] | 3 (Isomer 1) | Column: ALL 4.6 × 100 mm, 3 μm Mobile phase: 40% MeOH [1% NH₄OH] in CO₂ Flow rate: 12.5 mL/min Column temperature: 38.6 °C Retention time = 3.32 min | LC-MS: m/z = 469.1. (M + 1, ESI+). 1H NMR (400 MHz, ) δ 10.79 (s, 1H), 7.89 (d, J = 8.0 Hz, 2H), 7.88- 7.77 (m, 2H), 7.23 (s, 1H), 6.61 (s, 1H), 6.51 (s, 1H), 3.64 (s, 3H), 3.443- 3.37 (m, 2H), 2.61-2.56 (m, 2H), 2.38 (s, 3H), 2.33-2.27 (m, 2H), 2.26-2.13 (m, 2H), 1.78-1.51 (m, 4H), 1.49 (s, 3H). | | |
| Name and | | | Name and | | |
| Stucture | Ex. # | LCMS | Stucture | Ex. # | LCMS |
| 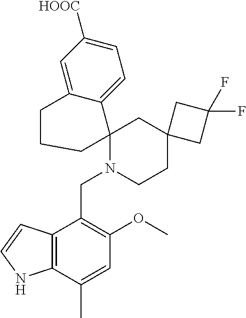 [SMILES: COc1cc(C)c2[nH]ccc2c1CN1CCC2(CC(F)(F)C2)CC12CCCc1cc(C(=O)O)ccc12] | 2 | 495.1 | 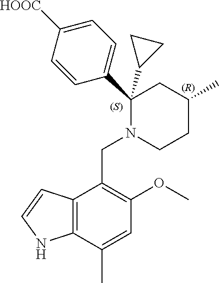 | 26-5 | 433.2 |
| 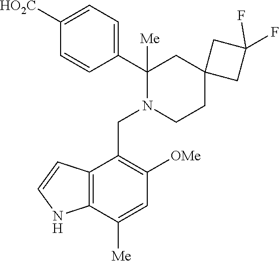 [SMILES: COc1cc(C)c2[nH]ccc2c1CN1CCC2(CC(F)(F)C2)CC1(C)c1ccc(C(=O)O)cc1] | 3 | 469.1 | 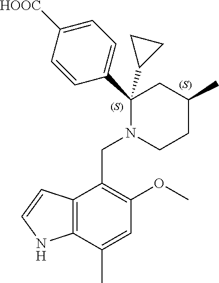 | 26-6 | 433.2 |
| 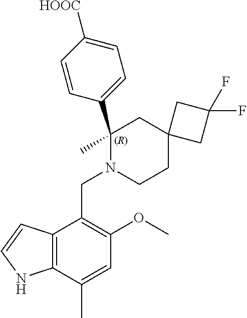 | 3-1 | 469.1 | 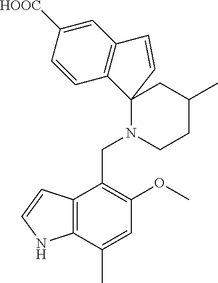 [SMILES: COc1cc(C)c2[nH]ccc2c1CN1CCC(C)CC12C=Cc1cc(C(=O)O)ccc12] | 27 | 417.2 |
| 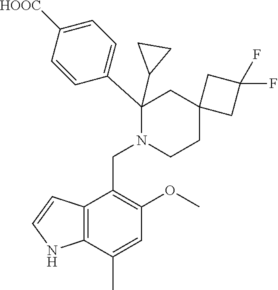 [SMILES: COc1cc(C)c2[nH]ccc2c1CN1CCC2(CC(F)(F)C2)CC1(c1ccc(C(=O)O)cc1)C1CC1] | 4 | 495.2 | 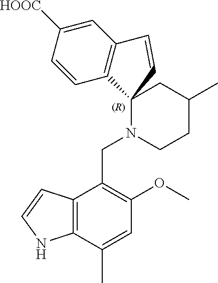 | 27-1 | 417.2 |
| 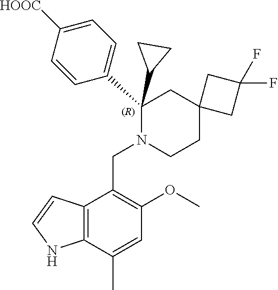 | 4-1 | 495.2 | 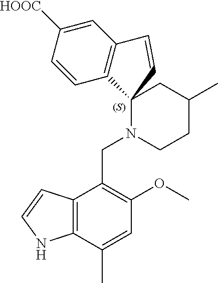 | 27-2 | 417.2 |
| 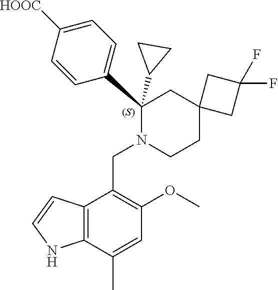 | 4-2 | 495.2 | 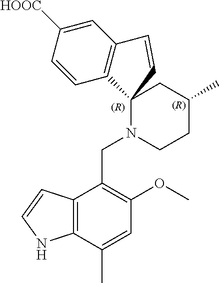 | 27-3 | 417.2 |
| 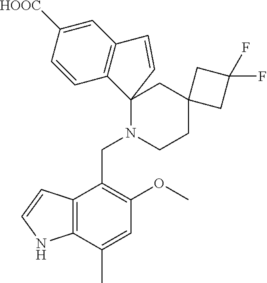 [SMILES: COc1cc(C)c2[nH]ccc2c1CN1CCC2(CC(F)(F)C2)CC12C=Cc1cc(C(=O)O)ccc12] | 5 | 479.2 | 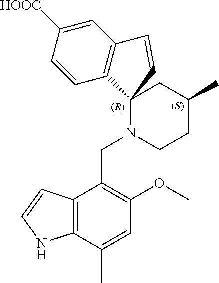 | 27-4 | 417.2 |
| 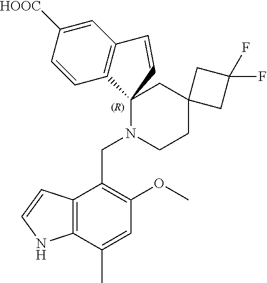 | 5-1 | 479.2 | 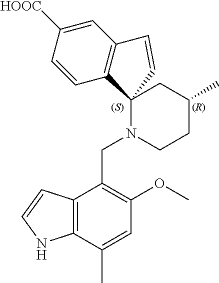 | 27-5 | 417.2 |
| 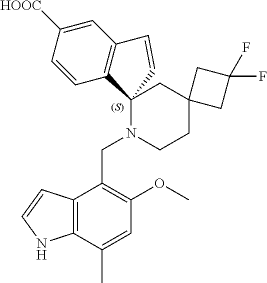 | 5-2 | 479.2 | 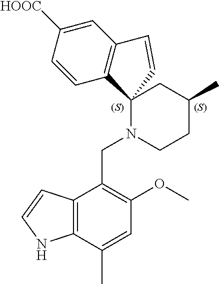 | 27-6 | 417.2 |
| 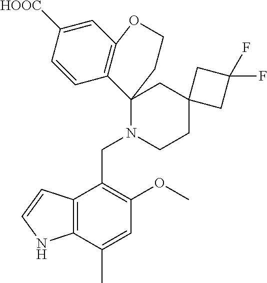 [SMILES: COc1cc(C)c2[nH]ccc2c1CN1CCC2(CC(F)(F)C2)CC12CCOc1cc(C(=O)O)ccc12] | 6 | 497.2 | 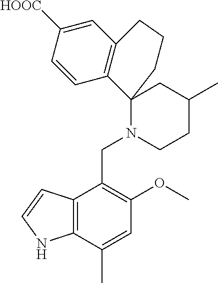 [SMILES: COc1cc(C)c2[nH]ccc2c1CN1CCC(C)CC12CCCc1cc(C(=O)O)ccc12] | 28 | 433.2 |
| 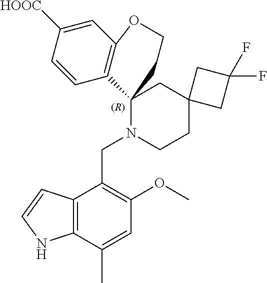 | 6-1 | 497.2 | 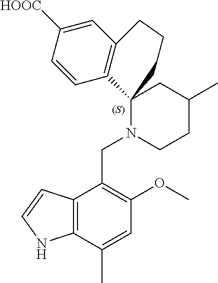 | 28-1 | 433.2 |
| 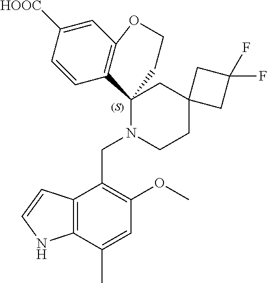 | 6-2 | 497.2 | 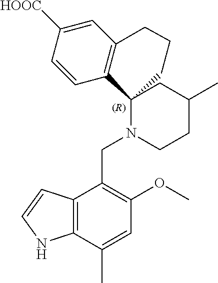 | 28-2 | 433.2 |
| 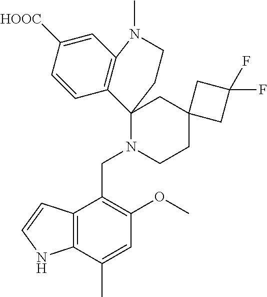 [SMILES: COc1cc(C)c2[nH]ccc2c1CN1CCC2(CC(F)(F)C2)CC12CCN(C)c1cc(C(=O)O)ccc12] | 7 | 509.3 | 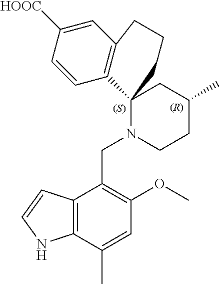 | 28-3 | 433.2 |
| 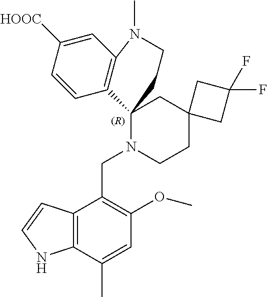 | 7-1 | 509.3 | 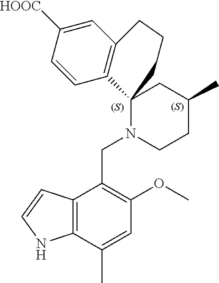 | 28-4 | 433.2 |
| 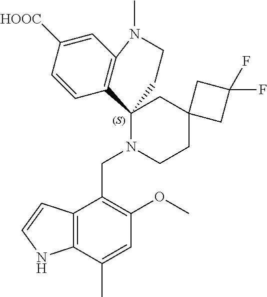 | 7-2 | 509.3 | 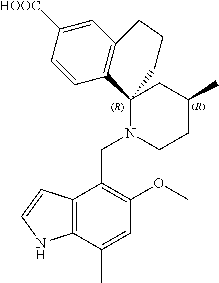 | 28-5 | 433.2 |
| 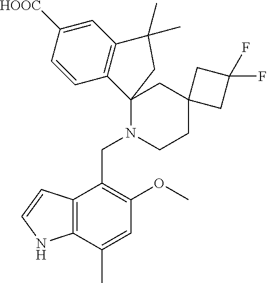 [SMILES: COc1cc(C)c2[nH]ccc2c1CN1CCC2(CC(F)(F)C2)CC12CC(C)(C)c1cc(C(=O)O)ccc12] | 8 | 509.3 | 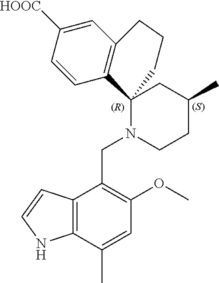 | 28-6 | 433.2 |
| 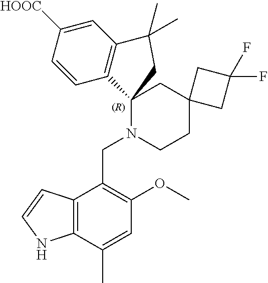 | 8-1 | 509.3 | 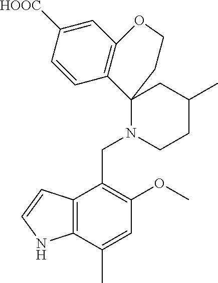 [SMILES: COc1cc(C)c2[nH]ccc2c1CN1CCC(C)CC12CCOc1cc(C(=O)O)ccc12] | 29 | 435.2 |
| 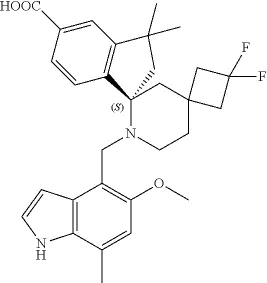 | 8-2 | 509.3 | 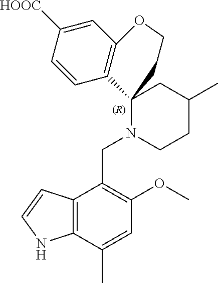 | 29-1 | 435.2 |
| 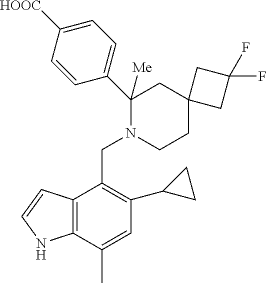 [SMILES: Cc1cc(C2CC2)c(CN2CCC3(CC(F)(F)C3)CC2(C)c2ccc(C(=O)O)cc2)c2cc[nH]c12] | 9 | 478.2 | 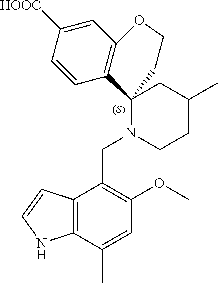 | 29-2 | 435.2 |
| 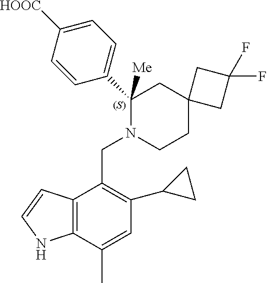 | 9-1 | 478.2 | 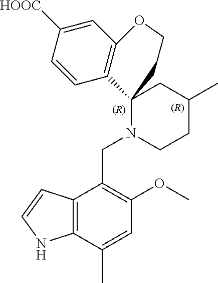 | 29-3 | 435.2 |
| 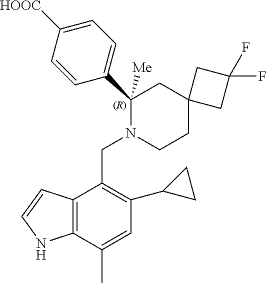 | 9-2 | 478.2 | 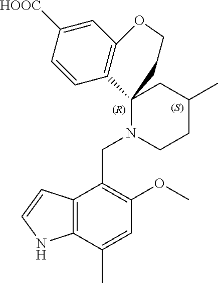 | 29-4 | 435.2 |
| 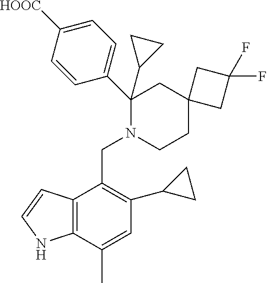 [SMILES: Cc1cc(C2CC2)c(CN2CCC3(CC(F)(F)C3)CC2(c2ccc(C(=O)O)cc2)C2CC2)c2cc[nH]c12] | 10 | 504.3 | 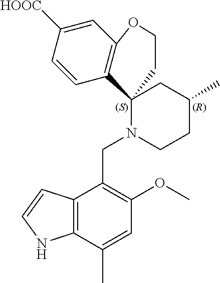 | 29-5 | 435.2 |
| 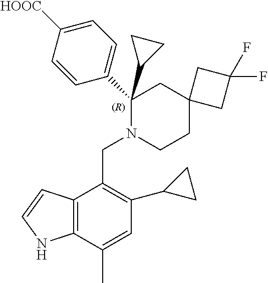 | 10-1 | 504.3 | 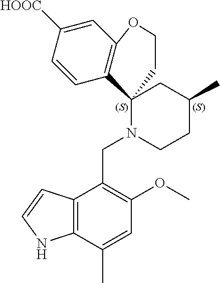 | 29-6 | 435.2 |
| 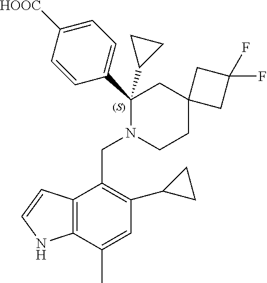 | 10-2 | 504.3 | 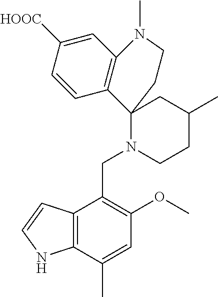 [SMILES: COc1cc(C)c2[nH]ccc2c1CN1CCC(C)CC12CCN(C)c1cc(C(=O)O)ccc12] | 30 | 448.3 |
| 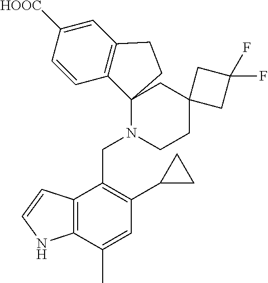 [SMILES: Cc1cc(C2CC2)c(CN2CCC3(CC(F)(F)C3)CC23CCc2cc(C(=O)O)ccc23)c2cc[nH]c12] | 11 | 490.2 | 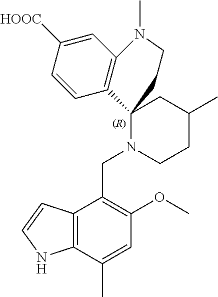 | 30-1 | 448.3 |
| 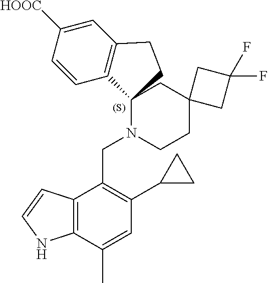 | 11-1 | 490.2 | 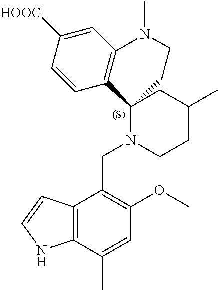 | 30-2 | 448.3 |
| 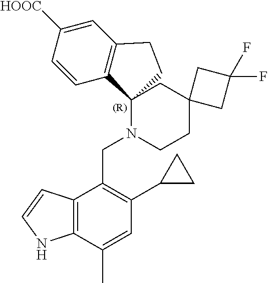 | 11-2 | 490.2 | 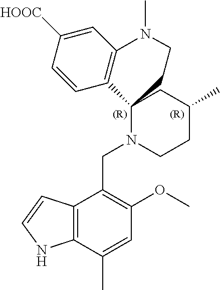 | 30-3 | 448.3 |
| 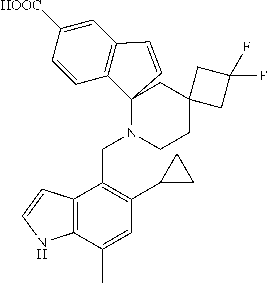 [SMILES: Cc1cc(C2CC2)c(CN2CCC3(CC(F)(F)C3)CC23C=Cc2cc(C(=O)O)ccc23)c2cc[nH]c12] | 12 | 488.2 | 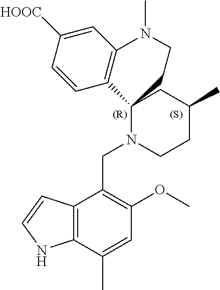 | 30-4 | 448.3 |
| 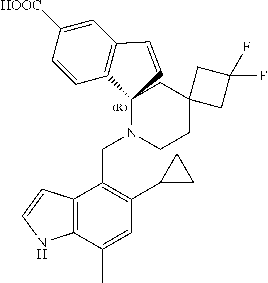 | 12-1 | 488.2 | 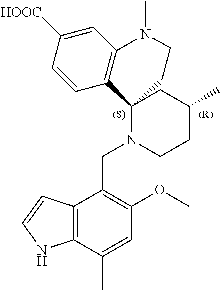 | 30-5 | 448.3 |
| 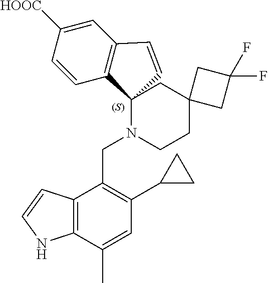 | 12-2 | 488.2 | 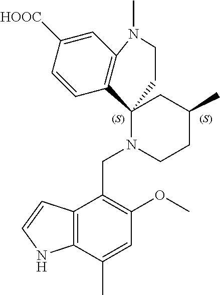 | 30-6 | 448.3 |
| 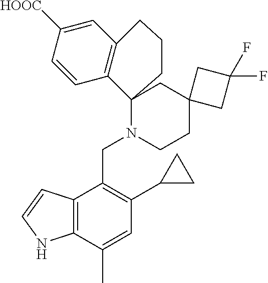 [SMILES: Cc1cc(C2CC2)c(CN2CCC3(CC(F)(F)C3)CC23CCCc2cc(C(=O)O)ccc23)c2cc[nH]c12] | 13 | 504.3 | 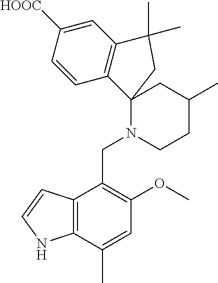 [SMILES: COc1cc(C)c2[nH]ccc2c1CN1CCC(C)CC12CC(C)(C)c1cc(C(=O)O)ccc12] | 31 | 447.3 |
| 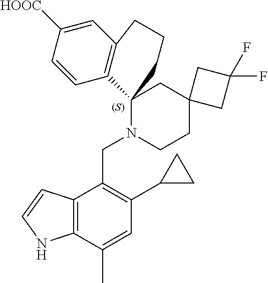 | 13-1 | 504.3 | 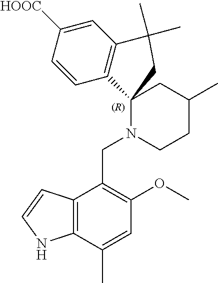 | 31-1 | 447.3 |
| 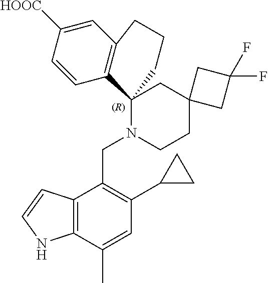 | 13-2 | 504.3 | 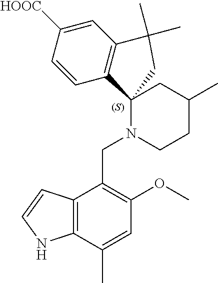 | 31-2 | 447.3 |
| 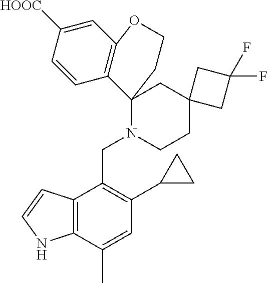 [SMILES: Cc1cc(C2CC2)c(CN2CCC3(CC(F)(F)C3)CC23CCOc2cc(C(=O)O)ccc23)c2cc[nH]c12] | 14 | 507.2 | 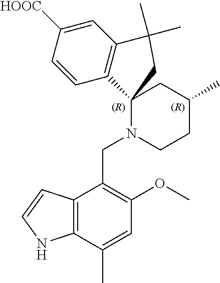 | 31-3 | 447.3 |
| 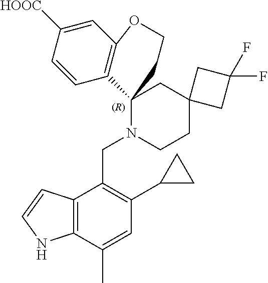 | 14-1 | 507.2 | 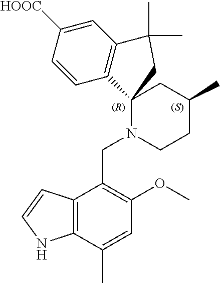 | 31-4 | 447.3 |
| 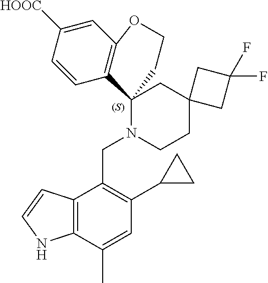 | 14-2 | 507.2 | 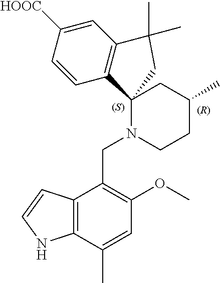 | 31-5 | 447.3 |
| 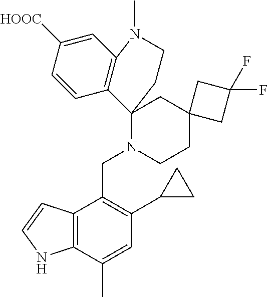 [SMILES: Cc1cc(C2CC2)c(CN2CCC3(CC(F)(F)C3)CC23CCN(C)c2cc(C(=O)O)ccc23)c2cc[nH]c12] | 15 | 520.3 | 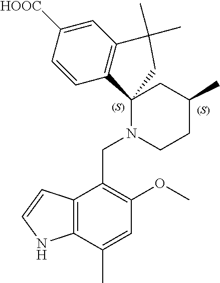 | 31-6 | 447.3 |
| 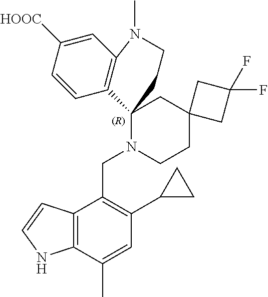 | 15-1 | 520.3 | 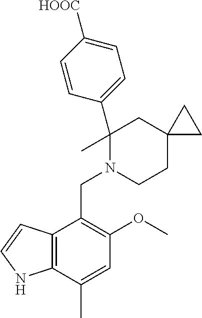 [SMILES: COc1cc(C)c2[nH]ccc2c1CN1CCC2(CC2)CC1(C)c1ccc(C(=O)O)cc1] | 32 | 419.2 |
| 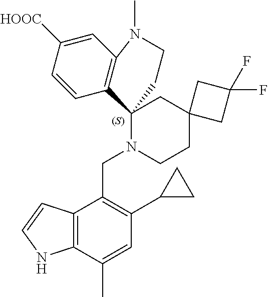 | 15-2 | 520.3 | 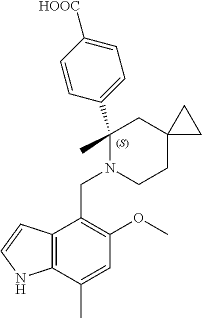 | 32-1 | 419.2 |
| 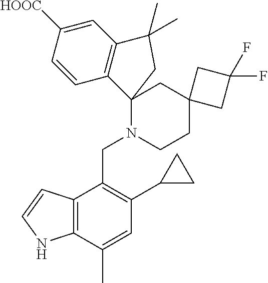 [SMILES: Cc1cc(C2CC2)c(CN2CCC3(CC(F)(F)C3)CC23CC(C)(C)c2cc(C(=O)O)ccc23)c2cc[nH]c12] | 16 | 519.3 | 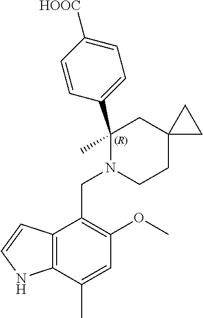 | 32-2 | 419.2 |
| 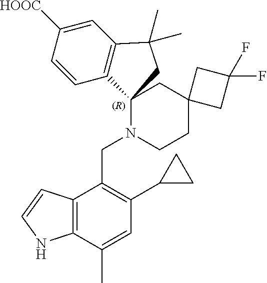 | 16-1 | 519.3 | 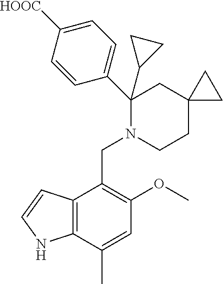 [SMILES: COc1cc(C)c2[nH]ccc2c1CN1CCC2(CC2)CC1(c1ccc(C(=O)O)cc1)C1CC1] | 33 | 445.2 |
| 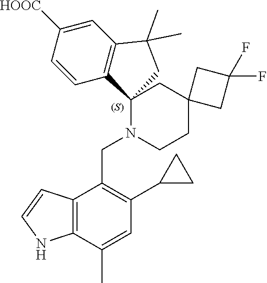 | 16-2 | 519.3 | 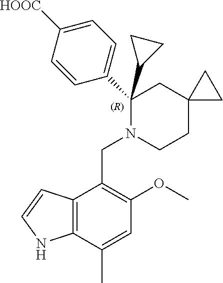 | 33-1 | 445.2 |
| 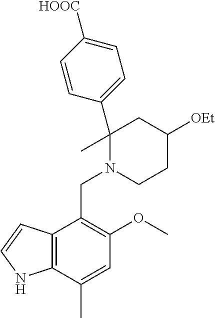 [SMILES: CCOC1CCN(Cc2c(OC)cc(C)c3[nH]ccc23)C(C)(c2ccc(C(=O)O)cc2)C1] | 17 | 437.2 | 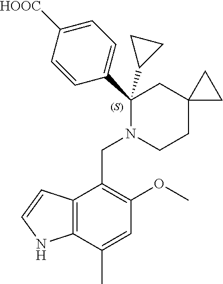 | 33-2 | 445.2 |
| 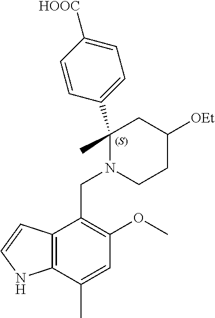 | 17-1 | 437.2 | 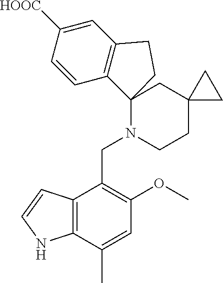 [SMILES: COc1cc(C)c2[nH]ccc2c1CN1CCC2(CC2)CC12CCc1cc(C(=O)O)ccc12] | 34 | 431.2 |
| 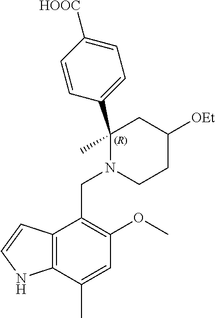 | 17-2 | 437.2 | 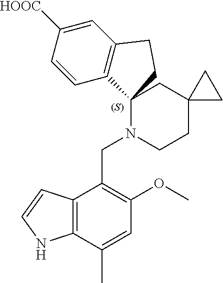 | 34-1 | 431.2 |
| 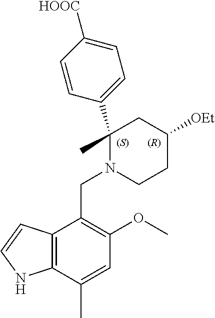 | 17-3 | 437.2 | 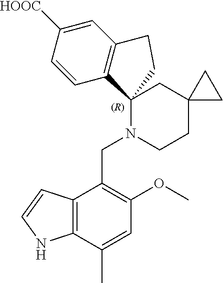 | 34-2 | 431.2 |
| 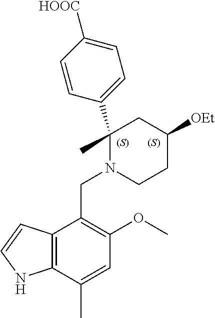 | 17-4 | 437.2 | 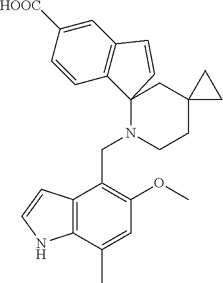 [SMILES: COc1cc(C)c2[nH]ccc2c1CN1CCC2(CC2)CC12C=Cc1cc(C(=O)O)ccc12] | 35 | 429.2 |
| 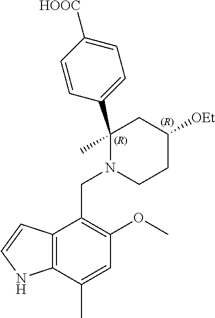 | 17-5 | 437.2 | 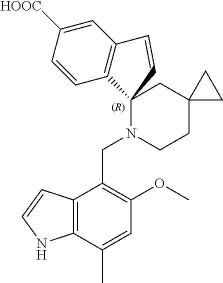 | 35-1 | 429.2 |
| 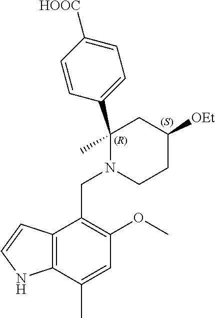 | 17-6 | 437.2 | 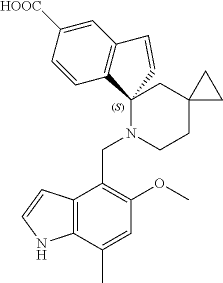 | 35-2 | 429.2 |
| 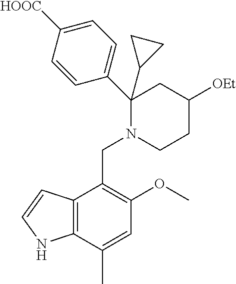 [SMILES: CCOC1CCN(Cc2c(OC)cc(C)c3[nH]ccc23)C(c2ccc(C(=O)O)cc2)(C2CC2)C1] | 18 | 436.3 | 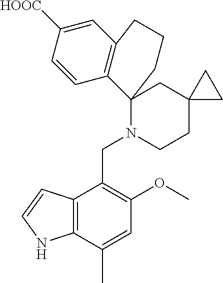 [SMILES: COc1cc(C)c2[nH]ccc2c1CN1CCC2(CC2)CC12CCCc1cc(C(=O)O)ccc12] | 36 | 445.2 |
| 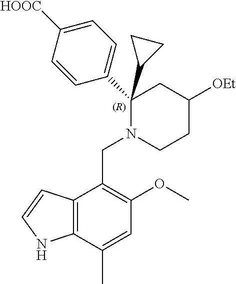 | 18-1 | 436.3 | 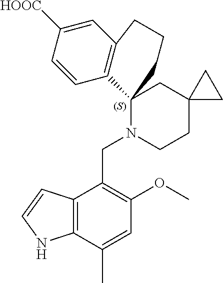 | 36-1 | 445.2 |
| 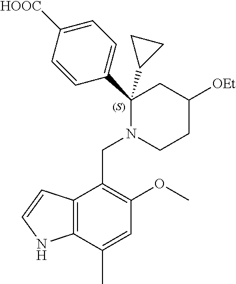 | 18-2 | 436.3 | 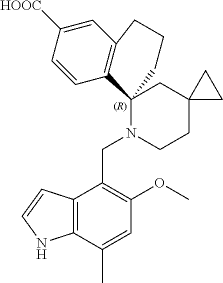 | 36-2 | 445.2 |
| 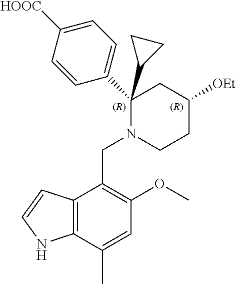 | 18-3 | 436.3 | 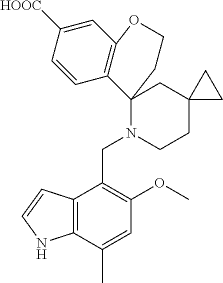 [SMILES: COc1cc(C)c2[nH]ccc2c1CN1CCC2(CC2)CC12CCOc1cc(C(=O)O)ccc12] | 37 | 447.2 |
| 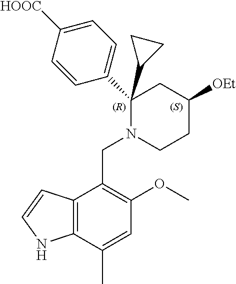 | 18-4 | 436.3 | 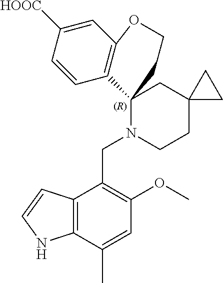 | 37-1 | 447.2 |
| 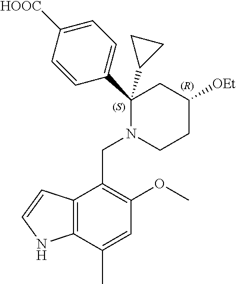 | 18-5 | 436.3 | 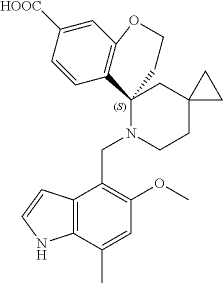 | 37-2 | 447.2 |
| 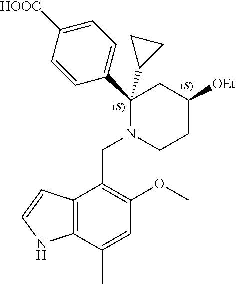 | 18-6 | 436.3 | 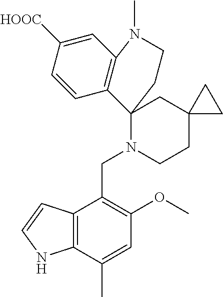 [SMILES: COc1cc(C)c2[nH]ccc2c1CN1CCC2(CC2)CC12CCN(C)c1cc(C(=O)O)ccc12] | 38 | 460.3 |
| 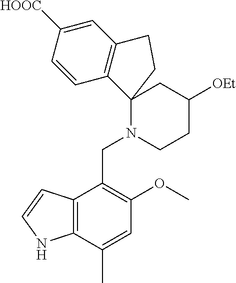 [SMILES: CCOC1CCN(Cc2c(OC)cc(C)c3[nH]ccc23)C2(CCc3cc(C(=O)O)ccc32)C1] | 19 | 449.2 | 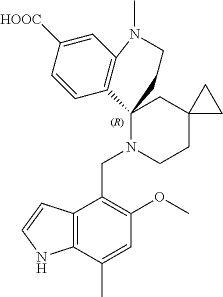 | 38-1 | 460.3 |
| 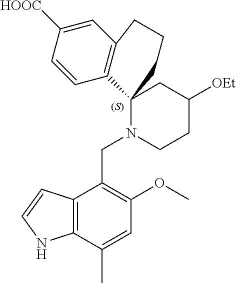 | 19-1 | 449.2 | 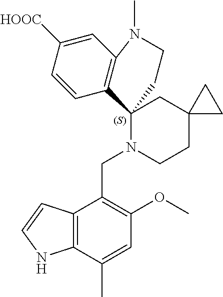 | 38-2 | 460.3 |
| 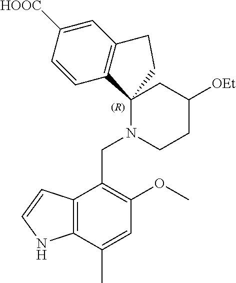 | 19-2 | 449.2 | 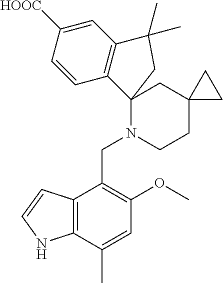 [SMILES: COc1cc(C)c2[nH]ccc2c1CN1CCC2(CC2)CC12CC(C)(C)c1cc(C(=O)O)ccc12] | 39 | 459.3 |
| 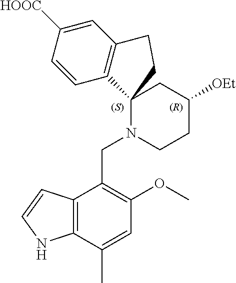 | 19-3 | 449.2 | 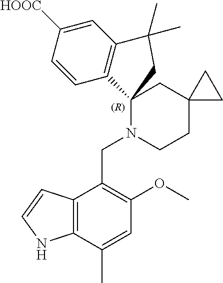 | 39-1 | 459.3 |
| 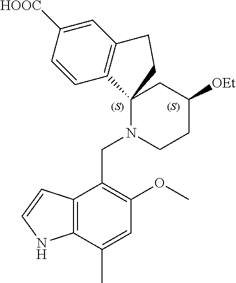 | 19-4 | 449.2 | 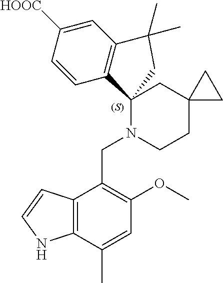 | 39-2 | 459.3 |
| 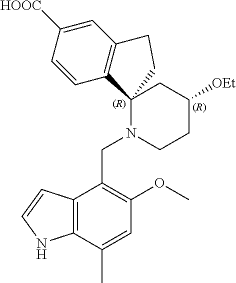 | 19-5 | 449.2 | 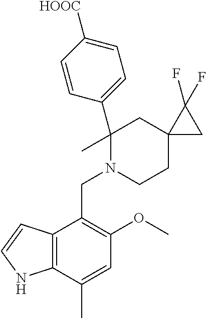 [SMILES: COc1cc(C)c2[nH]ccc2c1CN1CCC2(CC1(C)c1ccc(C(=O)O)cc1)CC2(F)F] | 40 | 455.2 |
| 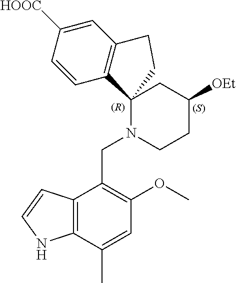 | 19-6 | 449.2 | 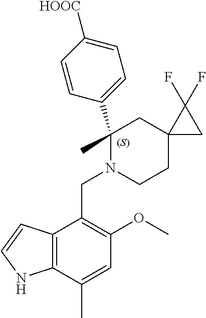 | 40-1 | 455.2 |
| 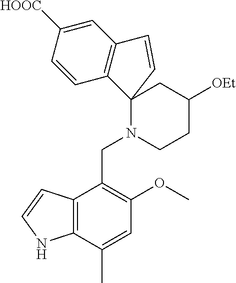 [SMILES: CCOC1CCN(Cc2c(OC)cc(C)c3[nH]ccc23)C2(C=Cc3cc(C(=O)O)ccc32)C1] | 20 | 447.2 | 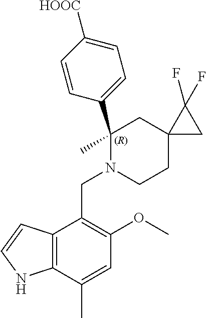 | 40-2 | 455.2 |
| 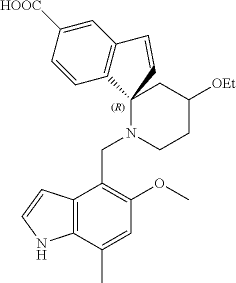 | 20-1 | 447.2 | 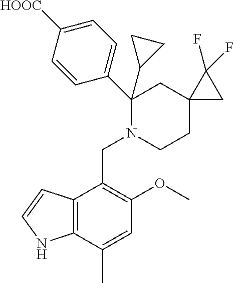 [SMILES: COc1cc(C)c2[nH]ccc2c1CN1CCC2(CC1(c1ccc(C(=O)O)cc1)C1CC1)CC2(F)F] | 41 | 481.2 |
| 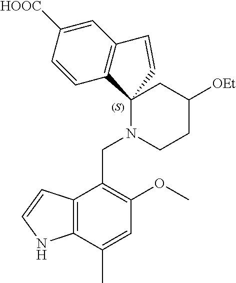 | 20-2 | 447.2 | 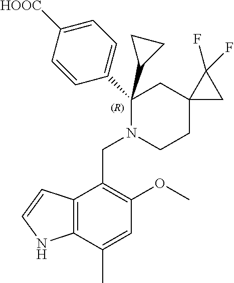 | 41-1 | 481.2 |
| 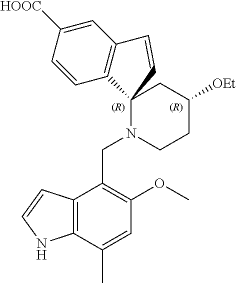 | 20-3 | 447.2 | 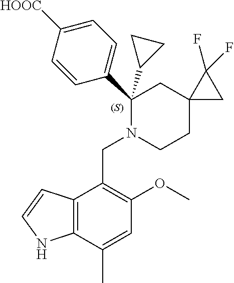 | 41-2 | 481.2 |
| 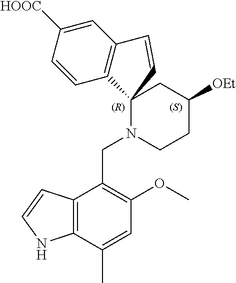 | 20-4 | 447.2 | 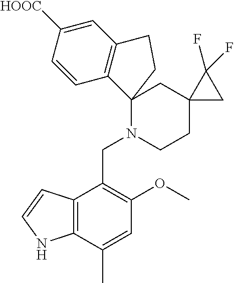 [SMILES: COc1cc(C)c2[nH]ccc2c1CN1CCC2(CC13CCc1cc(C(=O)O)ccc13)CC2(F)F] | 42 | 467.2 |
| 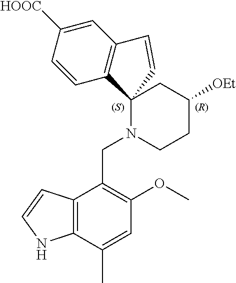 | 20-5 | 447.2 | 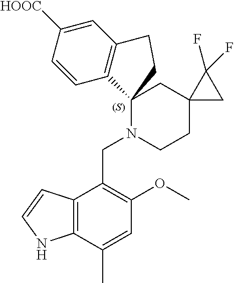 | 42-1 | 467.2 |
| 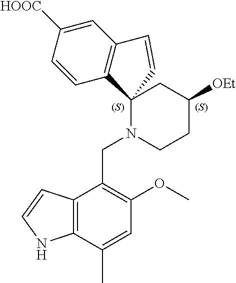 | 20-6 | 447.2 | 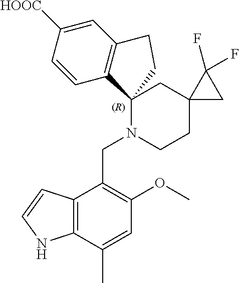 | 42-2 | 467.2 |
| 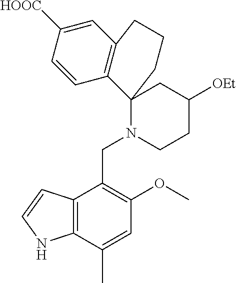 [SMILES: CCOC1CCN(Cc2c(OC)cc(C)c3[nH]ccc23)C2(CCCc3cc(C(=O)O)ccc32)C1] | 21 | 463.3 | 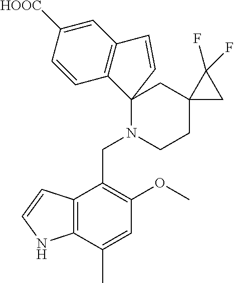 [SMILES: COc1cc(C)c2[nH]ccc2c1CN1CCC2(CC13C=Cc1cc(C(=O)O)ccc13)CC2(F)F] | 43 | 465.2 |
| 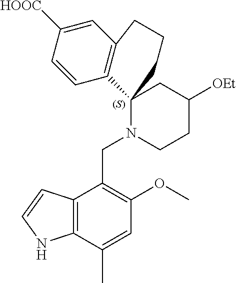 | 21-1 | 463.3 | 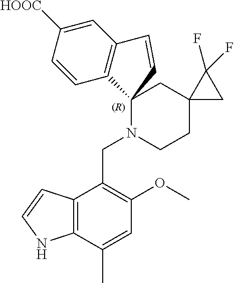 | 43-1 | 465.2 |
| 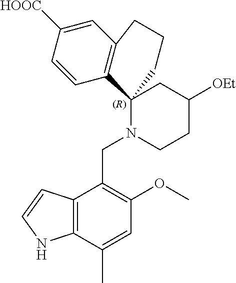 | 21-2 | 463.3 | 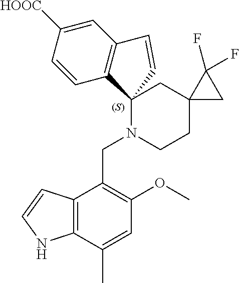 | 43-2 | 465.2 |
| 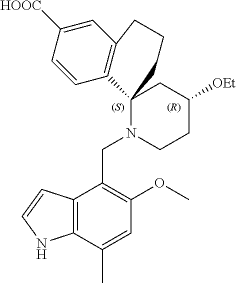 | 21-3 | 463.3 | 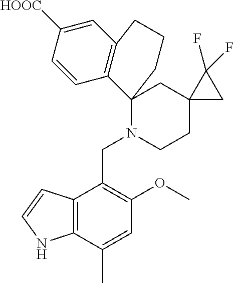 [SMILES: COc1cc(C)c2[nH]ccc2c1CN1CCC2(CC13CCCc1cc(C(=O)O)ccc13)CC2(F)F] | 44 | 481.2 |
| 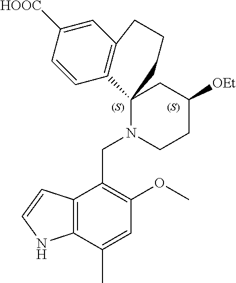 | 21-4 | 463.3 | 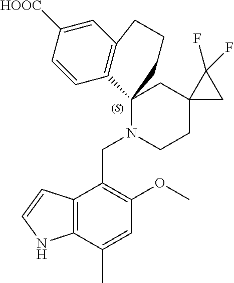 | 44-1 | 481.2 |
| 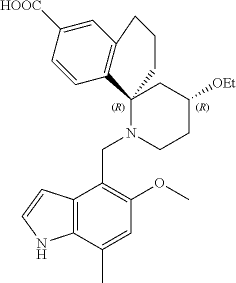 | 21-5 | 463.3 | 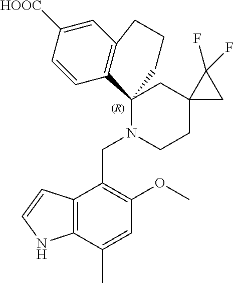 | 44-2 | 481.2 |
| 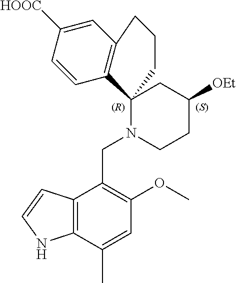 | 21-6 | 463.3 | 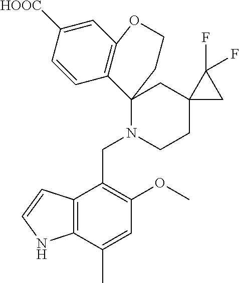 [SMILES: COc1cc(C)c2[nH]ccc2c1CN1CCC2(CC13CCOc1cc(C(=O)O)ccc13)CC2(F)F] | 45 | 483.2 |
| 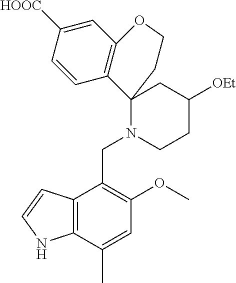 [SMILES: CCOC1CCN(Cc2c(OC)cc(C)c3[nH]ccc23)C2(CCOc3cc(C(=O)O)ccc32)C1] | 22 | 465.2 | 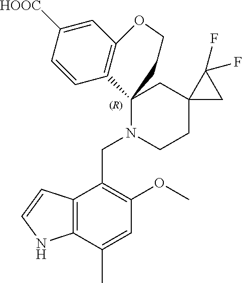 | 45-1 | 483.2 |
| 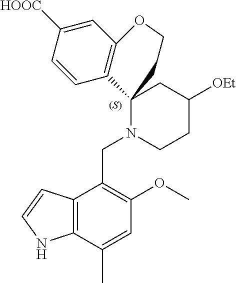 | 22-1 | 465.2 | 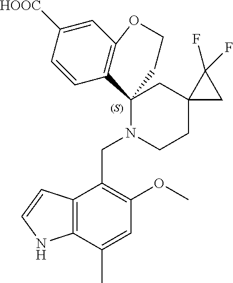 | 45-2 | 483.2 |
| 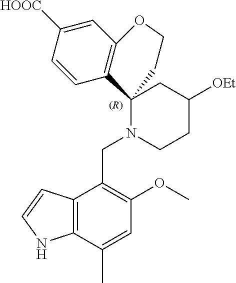 | 22-2 | 465.2 | 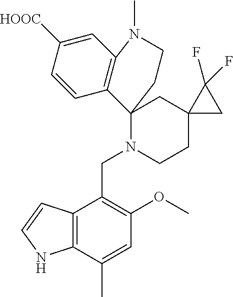 [SMILES: COc1cc(C)c2[nH]ccc2c1CN1CCC2(CC13CCN(C)c1cc(C(=O)O)ccc13)CC2(F)F] | 46 | 496.2 |
| 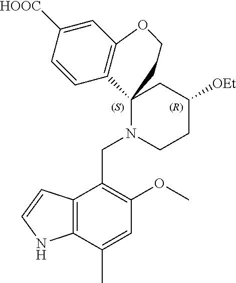 | 22-3 | 465.2 | 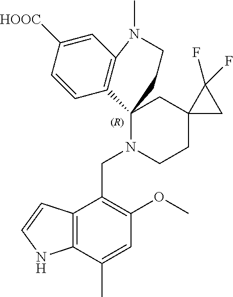 | 46-1 | 496.2 |
| 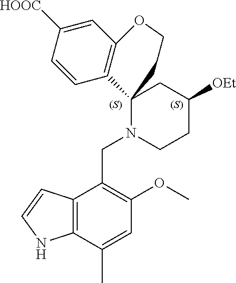 | 22-4 | 465.2 | 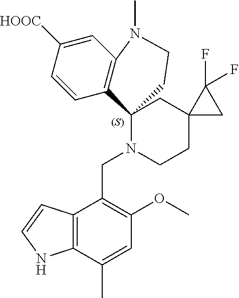 | 46-2 | 496.2 |
| 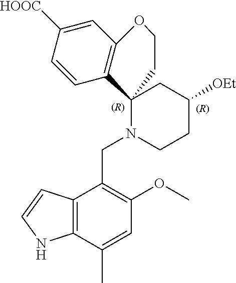 | 22-5 | 465.2 | 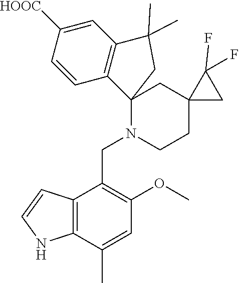 [SMILES: COc1cc(C)c2[nH]ccc2c1CN1CCC2(CC13CC(C)(C)c1cc(C(=O)O)ccc13)CC2(F)F] | 47 | 495.2 |
| 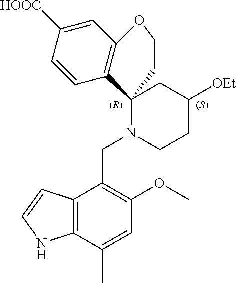 | 22-6 | 465.2 | 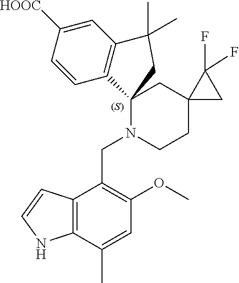 | 47-1 | 495.2 |
| 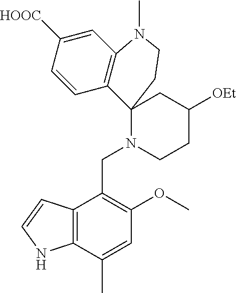 [SMILES: CCOC1CCN(Cc2c(OC)cc(C)c3[nH]ccc23)C2(CCN(C)c3cc(C(=O)O)ccc32)C1] | 23 | 478.3 | 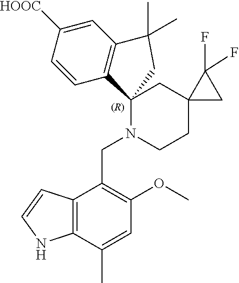 | 47-2 | 495.2 |
| 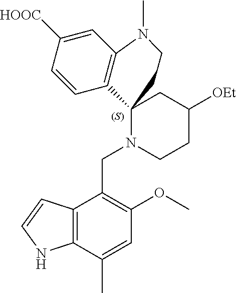 | 23-1 | 478.3 | 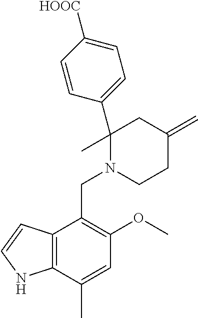 [SMILES: C=C1CCN(Cc2c(OC)cc(C)c3[nH]ccc23)C(C)(c2ccc(C(=O)O)cc2)C1] | 48 | 405.2 |
| 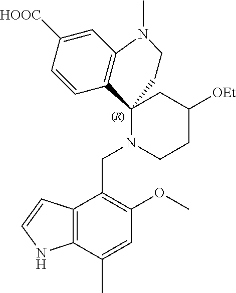 | 23-2 | 478.3 | 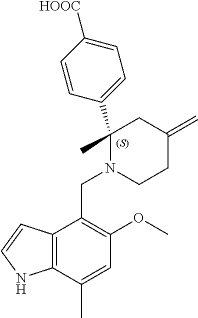 | 48-1 | 405.2 |
| 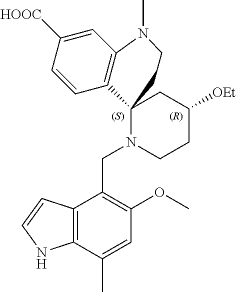 | 23-3 | 478.3 | 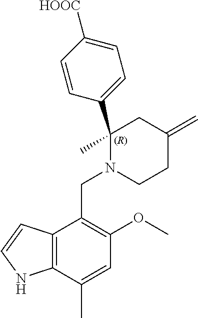 | 48-2 | 405.2 |
| 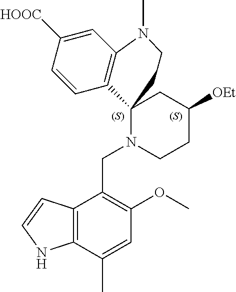 | 23-4 | 478.3 | 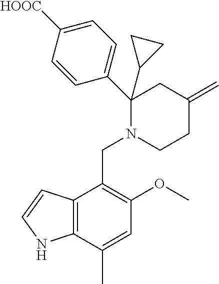 [SMILES: C=C1CCN(Cc2c(OC)cc(C)c3[nH]ccc23)C(c2ccc(C(=O)O)cc2)(C2CC2)C1] | 49 | 431.2 |
| 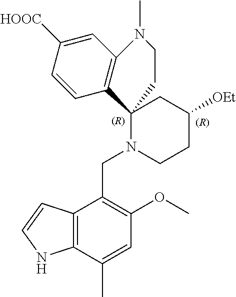 | 23-5 | 478.3 | 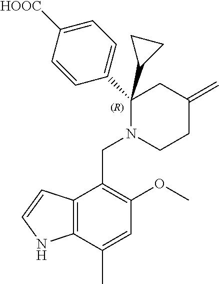 | 49-1 | 431.2 |
| 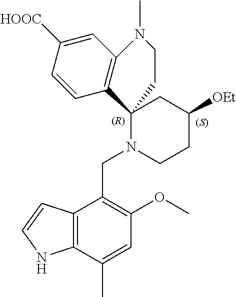 | 23-6 | 478.3 | 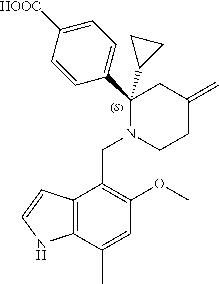 | 49-2 | 431.2 |
| 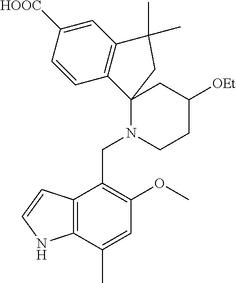 [SMILES: CCOC1CCN(Cc2c(OC)cc(C)c3[nH]ccc23)C2(C1)CC(C)(C)c1cc(C(=O)O)ccc12] | 24 | 477.3 | 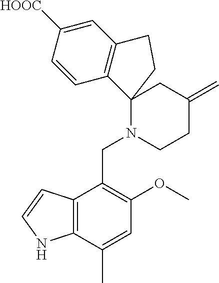 [SMILES: C=C1CCN(Cc2c(OC)cc(C)c3[nH]ccc23)C2(CCc3cc(C(=O)O)ccc32)C1] | 50 | 417.2 |
| 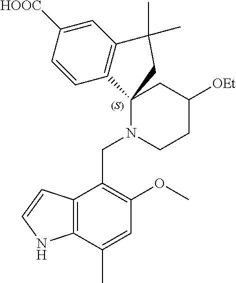 | 24-1 | 477.3 | 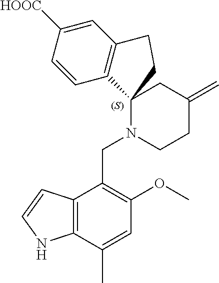 | 50-1 | 417.2 |
| 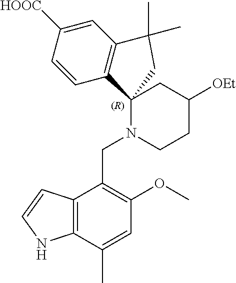 | 24-2 | 477.3 | 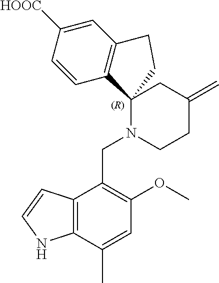 | 50-2 | 417.2 |
| 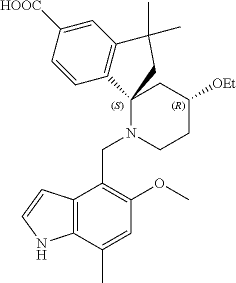 | 24-3 | 477.3 | 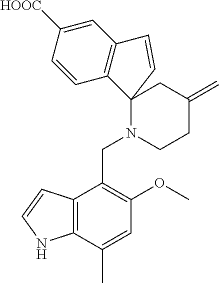 [SMILES: C=C1CCN(Cc2c(OC)cc(C)c3[nH]ccc23)C2(C=Cc3cc(C(=O)O)ccc32)C1] | 51 | 415.2 |
| 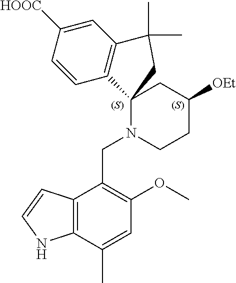 | 24-4 | 477.3 | 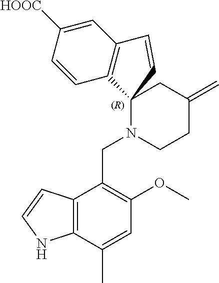 | 51-1 | 415.2 |
| 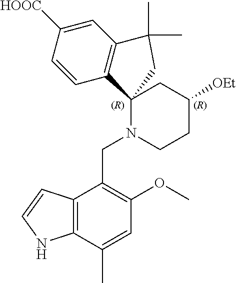 | 24-5 | 477.3 | 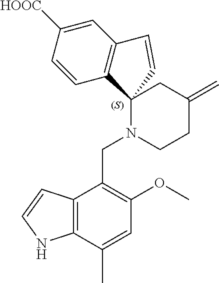 | 51-2 | 415.2 |
| 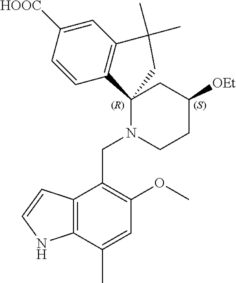 | 24-6 | 477.3 | 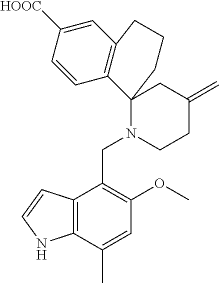 [SMILES: C=C1CCN(Cc2c(OC)cc(C)c3[nH]ccc23)C2(CCCc3cc(C(=O)O)ccc32)C1] | 52 | 431.2 |
| 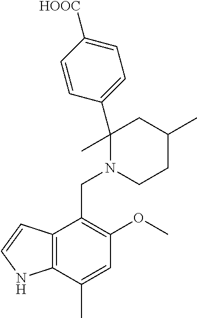 [SMILES: COc1cc(C)c2[nH]ccc2c1CN1CCC(C)CC1(C)c1ccc(C(=O)O)cc1] | 25 | 407.2 | 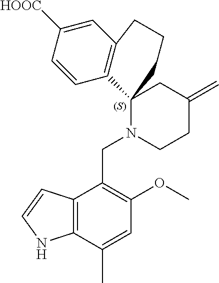 | 52-1 | 431.2 |
| 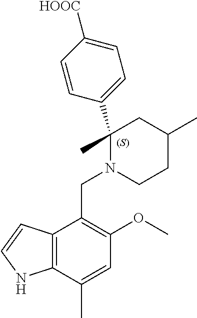 | 25-1 | 407.2 | 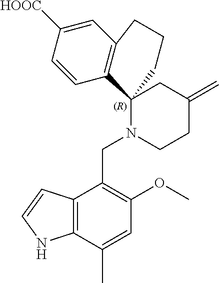 | 52-2 | 431.2 |
| 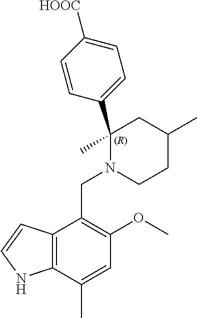 | 25-2 | 407.2 | 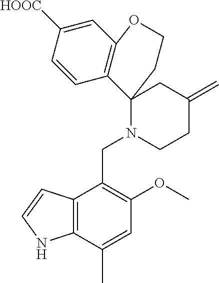 [SMILES: C=C1CCN(Cc2c(OC)cc(C)c3[nH]ccc23)C2(CCOc3cc(C(=O)O)ccc32)C1] | 53 | 433.2 |
| 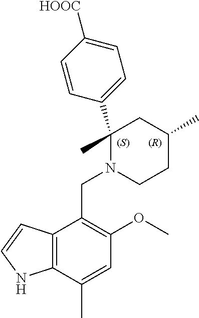 | 25-3 | 407.2 | 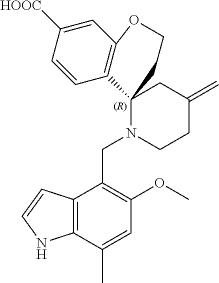 | 53-1 | 433.2 |
| 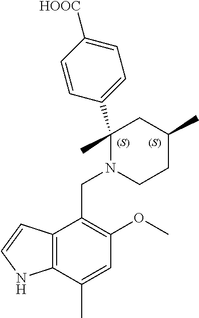 | 25-4 | 407.2 | 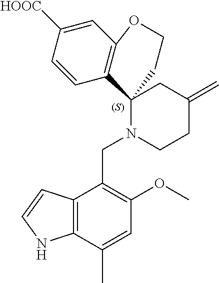 | 53-2 | 433.2 |
| 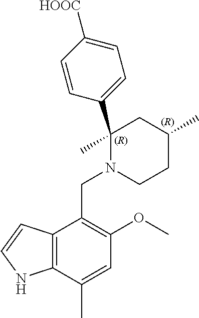 | 25-5 | 407.2 | 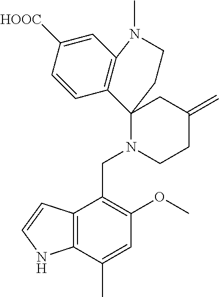 [SMILES: C=C1CCN(Cc2c(OC)cc(C)c3[nH]ccc23)C2(CCN(C)c3cc(C(=O)O)ccc32)C1] | 54 | 446.2 |
| 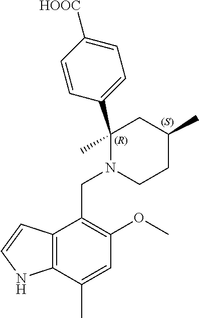 | 25-6 | 407.2 | 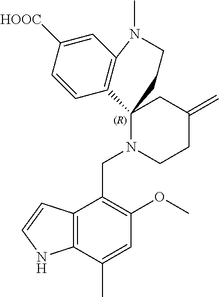 | 54-1 | 446.2 |
| 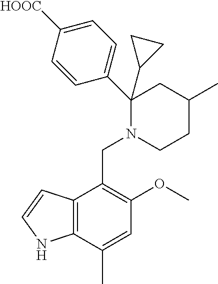 [SMILES: COc1cc(C)c2[nH]ccc2c1CN1CCC(C)CC1(c1ccc(C(=O)O)cc1)C1CC1] | 26 | 433.2 | 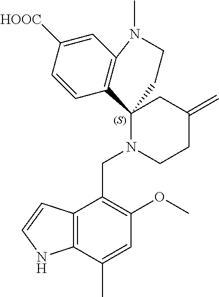 | 54-2 | 446.2 |
| 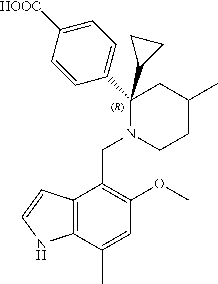 | 26-1 | 433.2 | 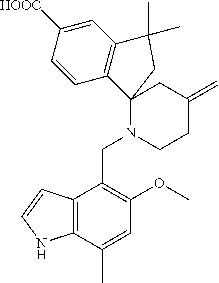 [SMILES: C=C1CCN(Cc2c(OC)cc(C)c3[nH]ccc23)C2(C1)CC(C)(C)c1cc(C(=O)O)ccc12] | 55 | 445.2 |
| 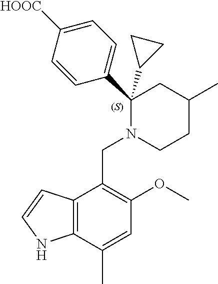 | 26-2 | 433.2 | 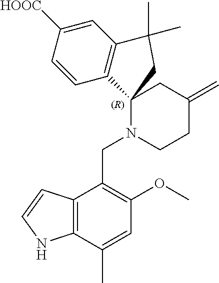 | 55-1 | 445.2 |
| 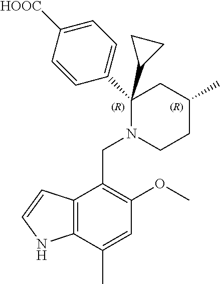 | 26-3 | 433.2 | 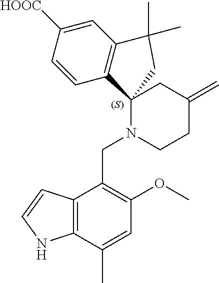 | 55-2 | 445.2 |
| 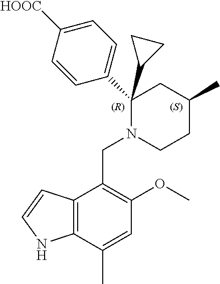 | 26-4 | 433.2 | | | |

### Biological Example 1. Factor B Binding Assay by TR-FRET

### Material and Reagents

1. Recombinant human Factor B catalytic domain (a.a. 470-764, C-terminal his-tagged, produced in-house)
2. 5× Kinase Buffer A (Thermo Fisher, CAT #PV3189)
3. LANCE Eu-W1024 Anti-6×His Antibody (PerkinElmer, CAT #AD0401)
4. Probe (TRFRET_tool 2, reported in WO 2015/009616)

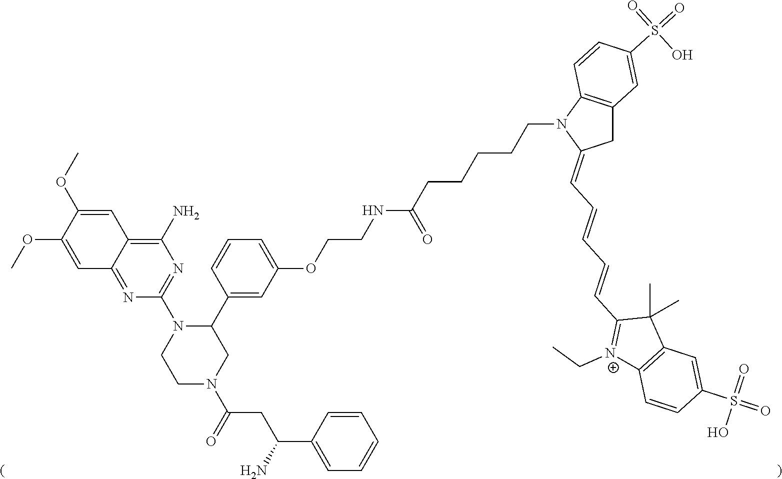 [SMILES: CC[N+]1=C(/C=C/C=C/C=C2\Cc3cc(S(=O)(=O)O)ccc3N2CCCCCC(=O)NCCOc2cccc(C3CN(C(=O)C[C@@H](N)c4ccccc4)CCN3c3nc(N)c4cc(OC)c(OC)cc4n3)c2)C(C)(C)c2cc(S(=O)(=O)O)ccc21]

5. DMSO (Thermo Fisher Scientific)
6. Compounds-10 mM stock in DMSO
7. Victor Nivo multimode plate reader (PerkinElmer)
8. OptiPlate-384, white opaque 384-well microplate (PerkinElmer, CAT #6007290)

### Experimental Procedure

Factor B binding affinity of each compound tested was determined using a time-resolved fluorescence resonance energy transfer (TR-FRET) technology. 10 nM recombinant his-tagged Factor B catalytic domain, varying concentrations of inhibitors, 4 nM LANCE Eu-W1024 Anti-6×His Antibody and 100 nM TRFRET_tool2 tracer was incubated in 1× Kinase Buffer A for 1 h. Measurement was performed in a reaction volume of 15 μL by adding 5 μL of the test compound, 5 μL of Factor B/antibody mixture and 5 μL of tracer into white opaque 384-well assay plates. The TR-FRET signal was read on a plate reader with an excitation wavelength of 340 nm and detection wavelengths of 615 and 665 nm. Binding affinity was determined for each compound by measuring TR-FRET signal at various concentrations of compound and plotting the relative fluorescence Emission Ratio (665 nm/615 nm) against the inhibitor concentration to estimate the IC₅₀ from [Compound] vs Emission Ratio using the four parameters dose-response inhibition curve with a variable slope model in GraphPad Prism.

The binding affinity to recombinant Factor B catalytic domain of the compounds of the present invention was determined by the above assay, and IC₅₀ values (nM) are shown in the following table.

TABLE 1
IC₅₀ values (nM) of the compounds in the
present invention against human Factor B.

| | | Factor B TR-FRET IC₅₀ |
|---|---|---|
| Example # | Isomer | (nM) |
| 1 | 1 | 1.05 |
| 2 | 1 | 2.01 |
| 3 | 1 | 1.14 |

Example compounds in present invention were tested in above assay and show IC₅₀ (nM) 0.5-250 nM against human Factor B.

### Biological Example 2. Target Residence Time of Factor B Inhibitors Determined by Surface Plasmon Resonance (SPR)

### Material and Reagents

1. Recombinant human Factor B catalytic domain (a. a. 470-764, C-terminal his-tagged, produced in-house)
2. PBS-P+ Buffer 10× (Cytiva, CAT #28995084)
3. Series S Sensor Chip NTA (Cytiva, CAT #BR100532)
4. Amine Coupling Kit (Cytiva, CAT #BR100050)
5. DMSO (Millipore Sigma, CAT #34869-1L)
6. Greiner 96 well plates, polypropylene (Sigma-Aldrich, CAT #M7310-100EA)
7. Microplate Foil, 96-well (Cytiva, CAT #28975816)
8. Biacore 8k (Cytiva)

### Experimental Procedure

Biacore 8k instrument was primed using 1×PBS-P+ buffer before docking a Cytiva NTA chip. Recombinant human Factor B catalytic domain were immobilized on a NTA chip to a level of about 5000 resonance units (RU) using 1×PBS-P+ buffer [20 mM phosphate buffer with 2.7 mM KCl, 137 mM NaCl, and 0.05% (v/v) Tween-20]. The protein ligand was further crosslinked to sensorchip surface by amine coupling kit. Immobilization and binding experiment were performed at room temperature.

After changing buffer to 1×PBS-P+ buffer with 2% (v/v) DMSO, a pre-run was performed for a period of at least 30 min at a flow rate of 30 μl/min to obtain a stable surface. The kinetic constants of the compounds were determined by single-cycle kinetics with six consecutive injections (or multi-cycle kinetics with eight consecutive injections) with an increasing compound concentration in ranges of 0.8-200 nM, 12.5-400 nM, 4.1-1,000 nM or 41-10,000 nM depending on the potency. Single-cycle kinetics experiments were performed with an association time of 60 s per concentration and a dissociation time of 300 s (or a dissociation time of 120 s for multi-cycle kinetics experiments). A flow rate of 30 μl/min was used. A blank run with the same conditions was performed before the compound was injected.

The SPR sensorgrams were analyzed with Biacore Insight Evaluation Software by using a method of double referencing. The resulting curve was fitted with a 1:1 binding model. The kinetic constants (ka, kd, KD) of replicates were averaged. Residence time (tR) was calculated from the dissociation constant kd with the formula tR=1/kd. Binding half-life (t1/2) was calculated from the dissociation constant kd with the formula t1/2=ln 2/kd

TABLE 2
Kinetic constants and target residence time of the compounds
in the present invention against human Factor B.

| Example # | Isomer | K_{D} (nM) | Residence time (s) | t_{1/2} (s) |
|---|---|---|---|---|
| 1 | 1 | 0.9 | 1017 | 705 |
| 2 | 1 | 2.2 | 1175 | 814 |
| 3 | 1 | 1.73 | 1092 | 757 |

Example compounds in present invention were tested in above assay and show K_{D} (nM) 0.5-250 nM against human Factor B.

### Biological Example 3: Inhibition of Human MAC Deposition

To quantify the inhibitory effects of compounds on complement protein deposition following LPS activation of the alternative complement pathway, the AP deposition assay was employed. Test concentration of the compounds started at 11.1 μM or 3.70 UM and were diluted 3-fold in DMSO to produce 8 concentration points, with each concentration tested in technical triplicates. Human serum was combined with test compounds in GVB buffer containing 5 mM MgCl2 10 mM EGTA, and was added to LPS coated black Maxisorp plates. After a period of incubation, the plate was inverted, and ELISA detection of complement proteins bound to LPS was performed. A primary antibody that detected human C5b-9 was used, followed by a compatible HRP conjugated secondary antibody. Peroxidase activity was detected using the Quantablu Fluorogenic Peroxidase substrate kit and fluorescence was measured at 340 nm/435 nm using the i3x plate reader to determine the quantity of alternative pathway complement protein deposition.

TABLE 3
Inhibition of MAC (Membrane Attack Complex) formation

| Example # | Isomer | IC₅₀ (nM) |
|---|---|---|
| 1 | 1 | 147 |
| 2 | 1 | 129 |
| 3 | 1 | 193 |

Example compounds in present invention were tested in above assay and show IC₅₀ (nM) 125-5000 nM against human Factor B.

### Biological Example 4: Inhibition of AP Dependent Hemolysis

To quantify the inhibitory effects of compounds on the hemolytic activity of the alternative complement pathway, the AP hemolytic assay was employed. Test concentration of the compounds started at 100 μM and were diluted 3-fold in DMSO to produce 8-11 concentration points, with each concentration tested in technical triplicates. Human serum was added to rabbit reticulocytes in a solution of GVB buffer with MgCl2 and EGTA. After a period of incubation at 37 °C, spontaneous lysis of rabbit erythrocytes due to serum AP complement activity was quantified using OD405 absorbance, measured using the i3x plate reader.

TABLE 4
Inhibition of rabbit reticulocyte hemolysis

| Example # | Isomer | IC₅₀ (nM) |
|---|---|---|
| 1 | 1 | 220 |
| 2 | 1 | 283 |
| 3 | 1 | 252 |

Example compounds in present invention were tested in above assay and show IC₅₀ (nM) 125-5000 nM against human Factor B.

### Biological Example 5. Pharmacokinetic Studies in Rats

Three-month-old brown Norway rats were administered the Example compound intravenously (0.5 mg/kg dose, formulation: 10% 1,2-Propanediol+25% (20% solutol HS 15 in water)+65% phosphate buffered saline at 0.1 mg/mL) and via oral gavage at 2 mg/kg dose as a clear solution (0.5% MC+0.5% Tween 80 in water at 1 mg/mL). Plasma was collected from rats per time point at 0.25, 0.5, 1, 6, and 24 h after administration. The concentrations of the test article were measured in plasma by HPLC-MS/MS, two individual plasma samples at each time point. Chromatographic separation was carried out on Waters BEH C18 Column (2.1×50 mm, 1.7 μm) column (MAC-MOD Analytical, Chadds Ford, PA), using a gradient elution method with water and acetonitrile, both containing 0.025% formic acid-1 mM NH₄OAc. Mass spectrometric measurements in positive electrospray ionization were directed at quantifying the mass transition with [M+H]⁺ as the precursor ion on API6500, triple quadruple mass spectrometer (Sciex, Framingham, MA). The relevant pharmacokinetic parameters were estimated using noncompartmental methods using WinNonlin (Enterprise, version 8.2).

TABLE 5
The results of the PK studies are
in rats (0.5 mpk IV and 2 mpk PO)

| | 0.5 MPK IV | | | Plasma |
|---|---|---|---|---|
| | arm CL | T½ (h) - | | AUC₀₋ₗₐₛₜ |
| | (mL/min/Kg) | 2 MPK PO | % F | (nM · h) |
| Example 1 isomer 1 | 1.3 | 8.5 | 56 | 25424 |

Conclusion: The present invention describes compounds that are potent factor B inhibitors with desirable physiochemical properties. The described compounds may serve as useful as therapeutic agents for claims made above.

1-18. (canceled)

19. A compound of formula (I): 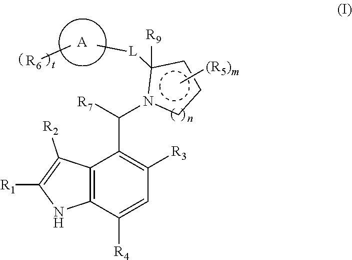
or a tautomer, cis- or trans-isomer, mesomer, racemate, enantiomer, diastereomer, or mixture thereof, or a pharmaceutically acceptable salt, solvate, or prodrug thereof,
wherein, 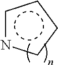
is a saturated or unsaturated ring;
A is C₆₋₁₀ aryl or 5-10 membered heteroaryl;
L is bond or CH₂;
Rₐ and R_{b} are independently selected from the group consisting of hydrogen, deuterium, halogen, amino, cyano, hydroxy, alkyl, alkoxy, alkylthio, haloalkyl, hydroxyalkyl, cycloalkyl, heterocyclyl, aryl and heteroaryl;
R₁ and R₂ are independently selected from the group consisting of hydrogen, deuterium, halogen, amino, cyano, hydroxy, alkyl, alkoxy, alkylthio, haloalkyl and hydroxyalkyl;
R₃ and R₄ are independently selected from the group consisting of hydrogen, deuterium, halogen, amino, cyano, hydroxy, C₁₋₆ alkyl, C₁₋₆ alkoxy, C₁₋₆ alkylthio, C₁₋₆ haloalkyl, C₁₋₆ haloalkenyl C₁₋₆ hydroxyalkyl, deuterated C₁₋₆ alkoxy, C₁₋₆ haloalkoxy, C₃₋₆ cycloalkyl, 4-10 membered heterocyclyl, C₆₋₁₀ aryl, 5-10 membered heteroaryl, C₃₋₆ cycloalkyloxy, 4-10 membered heterocyclyloxy, C₆₋₁₀ aryloxy and 5-10 membered heteroaryloxy, optionally the C₁₋₆ alkyl, C₁₋₆ alkoxy, C₁₋₆ alkylthio, C₁₋₆ haloalkyl, C₁₋₆ hydroxyalkyl, deuterated C₁₋₆ alkoxy, C₁₋₆ haloalkoxy substituted with one or more substituents selected from deuterium, halogen, amino, cyano, hydroxy, C₁₋₆ alkyl, C₁₋₆ alkoxy, C₁₋₆ alkylthio, C₁₋₆ haloalkyl, C₁₋₆ hydroxyalkyl, C₃₋₆ cycloalkyl, 4-10 membered heterocyclyl, C₆₋₁₀ aryl and 5-10 membered heteroaryl;
two of R₅ together with the C atom to which they are attached form C₃₋₆ cycloalkyl or 4-6 membered heterocyclyl containing 1, 2 or 3 heteroatoms selected from N, O or S, optionally substituted with one or more substituents selected from hydrogen, deuterium, halogen, amino, cyano, hydroxy, C₁₋₆ alkyl, C₁₋₆ alkoxy, C₁₋₆ alkylC₁₋₆ alkoxy, C₁₋₆ alkoxyC₁₋₆ alkyl, C₁₋₆ alkylthio, C₁₋₆ haloalkyl and C₁₋₆ hydroxyalkyl;
or, R₅ is independently selected from the group consisting of
hydrogen, deuterium, halogen, amino, cyano, hydroxy, C₁₋₆ alkyl, C₂₋₆ alkenyl, C₂₋₆ alkynyl, C₁₋₆ alkoxy, C₁₋₆ alkylthio, C₁₋₆ haloalkyl, C₁₋₆ hydroxyalkyl, C₃₋₈ cycloalkyl, 4-10 membered heterocyclyl, C₅₋₁₀ aryl and 5-10 membered heteroaryl, optionally the C₁₋₆ alkyl, C₁₋₆ alkoxy, C₁₋₆ alkylthio, C₁₋₆ haloalkyl, C₁₋₆ hydroxyalkyl, C₃₋₈ cycloalkyl, 4-10 membered heterocyclyl, C₅₋₁₀ aryl and 5-10 membered heteroaryl substituted with one or more substituents selected from deuterium, halogen, amino, cyano, hydroxy, C₁₋₆ alkyl, C₁₋₆ alkoxy, C₁₋₆ alkylthio, C₁₋₆ haloalkyl and C₁₋₆ hydroxyalkyl;
or, two of R₅ together with the C atom to which they are attached form a carbon-carbon double bond;
R₉ taken together with one of R₆ form a saturated or unsaturated C₃₋₇ cycloalkyl or a saturated or unsaturated 3-7 membered heterocyclyl containing 1, 2 or 3 heteroatoms selected from N, O or S, optionally the C₃₋₇ cycloalkyl or 3-7 membered heterocyclyl is substituted with 1 or 2 substituents selected from hydrogen, deuterium, halogen, amino, cyano, hydroxy, C₁₋₆ alkyl, C₁₋₆ alkoxy, C₁₋₆ alkylalkoxy, C₁₋₆ alkoxyalkyl, C₁₋₆ alkylthio, C₁₋₆ haloalkyl and C₁₋₆ hydroxyalkyl, and another R₆ is selected from the group consisting of hydrogen, deuterium, halogen, amino, cyano, hydroxy, C₁₋₃ alkyl, C₁₋₆ alkoxy, C₁₋₆ alkylthio, C₁₋₆ haloalkyl, C₁₋₆ hydroxyalkyl, C₃₋₈ cycloalkyl, 4-10 membered heterocyclyl, C₅₋₁₀ aryl and 5-10 membered heteroaryl, —(CH₂)ᵣC₁₋₆ alkoxy, —(CH₂)ᵣC(O)OH, —S(O)NHC₁₋₆ alkyl, —SO₂C₁₋₆ alkyl, —C(O)NHSO₂C₁₋₆ alkyl and —SO₂NHC(O)C₁₋₆ alkyl;
or, R₆ is selected from the group consisting of hydrogen, deuterium, halogen, amino, cyano, hydroxy, C₁₋₆ alkyl, C₁₋₆ alkoxy, C₁₋₆ alkylthio, C₁₋₆ haloalkyl, C₁₋₆ hydroxyalkyl, C₃₋₈ cycloalkyl, 4-10 membered heterocyclyl, C₅₋₁₀ aryl and 5-10 membered heteroaryl, —(CH₂)ᵣC₁₋₆ alkoxy, —(CH₂)ᵣC(O)OH, —S(O)NHC₁₋₆ alkyl, —SO₂C₁₋₆ alkyl, —C(O)NHSO₂C₁₋₆ alkyl and —SO₂NHC(O)C₁₋₆ alkyl;
R₉ is selected from the group consisting of deuterium, halogen, amino, cyano, hydroxy, C₁₋₆ alkyl, C₁₋₆ alkoxy, C₁₋₆ alkylthio, C₁₋₆ haloalkyl, C₁₋₆ hydroxyalkyl, C₃₋₈ cycloalkyl and 4-10 membered heterocyclyl containing 1, 2 or 3 heteroatoms selected from N, O or S;
R₇ is selected from the group consisting of hydrogen, deuterium, halogen, amino, cyano, hydroxy, alkyl, alkoxy, alkylthio, haloalkyl and hydroxyalkyl;
R₈ is selected from the group consisting of deuterium, halogen, amino, cyano, hydroxy, alkyl, alkoxy, alkylthio, haloalkyl and hydroxyalkyl;
p is 1, 2 or 3;
tis 1, 2 or 3;
m is 1, 2 or 3;
n is 0, 1, 2 or 3; and,
r is 0, 1, 2 or 3;
Provided that, R₃ and R₄ are not simultaneously methyl.

20. The compound of claim 19, or a tautomer, cis- or trans-isomer, mesomer, racemate, enantiomer, diastereomer, or mixture thereof, or a pharmaceutically acceptable salt, solvate, or prodrug thereof, wherein A is phenyl, benzocycloalkyl, or 5-8 membered heteroaryl containing 1, 2 or 3 of N heteroatoms;
preferably, A is

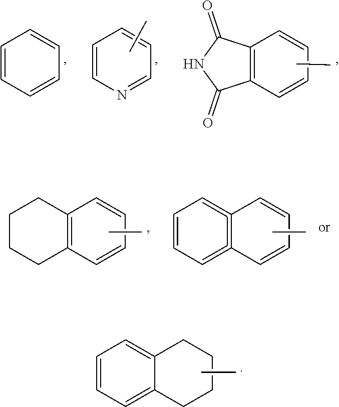

21. The compound of claim 19, or a tautomer, cis- or trans-isomer, mesomer, racemate, enantiomer, diastereomer, or mixture thereof, or a pharmaceutically acceptable salt, solvate, or prodrug thereof, wherein R₁ and R₂ are independently selected from hydrogen.

22. The compound of claim 19, or a tautomer, cis- or trans-isomer, mesomer, racemate, enantiomer, diastereomer, or mixture thereof, or a pharmaceutically acceptable salt, solvate, or prodrug thereof, wherein R₃ and R₄ are independently selected from the group consisting of deuterium, halogen, C₁₋₃ alkyl, C₁₋₃ alkoxy, deuterated C₁₋₃ alkoxy, C₁₋₃ haloalkoxy, C₃₋₆ cycloalkyl and C₃₋₆ cycloalkyloxy, optionally the C₁₋₃ alkyl, C₁₋₃ alkoxy, deuterated C₁₋₃ alkoxy, C₁₋₃ haloalkoxy substituted with one or more substituents selected from C₃₋₆ cycloalkyl, 4-6 membered heterocyclyl, C₆₋₁₀ aryl and 5-10 membered heteroaryl.

23. The compound of claim 19, or a tautomer, cis- or trans-isomer, mesomer, racemate, enantiomer, diastereomer, or mixture thereof, or a pharmaceutically acceptable salt, solvate, or prodrug thereof, wherein R₆ is —F, —OMe, —CH₂OH, —CH₂OCH₃, —CH₂F, —CF₂H, —CF₃, —COOH, —C(O)NHSO₂CH₃, —S(O)NHCH₃, or 5-6 membered heterocyclyl containing 1-3 of heteroatom selected from N, O and S, or 5-6 membered heteroaryl containing 1-3 of heteroatom selected from N, O and S.

24. The compound of claim 19, or tautomer, pharmaceutically acceptable salt thereof, wherein R₉ taken together with one of R₆ form a saturated or unsaturated C4-6 cycloalkyl or a saturated or unsaturated 4-7 membered heterocyclyl containing 1, 2 or 3 heteroatoms selected from N, O or S, optionally the saturated or unsaturated C4-6 cycloalkyl or saturated or unsaturated 4-7 membered heterocyclyl is substituted with 1 or 2 substituents selected from hydrogen, deuterium, halogen, amino, cyano, hydroxy, C₁₋₃ alkyl, C₁₋₃ alkoxy, C₁₋₃ alkylalkoxy, C₁₋₃ alkoxyalkyl, C₁₋₃ alkylthio, C₁₋₃ haloalkyl and C₁₋₃ hydroxyalkyl, and another R₆ is selected from the group consisting of F, —OMe, —CH₂OH, —CH₂OCH₃, —CH₂F, —CF₂H, —CF₃, —COOH, —C(O)NHSO₂CH₃, —S(O)NHCH₃;
or, R₉ is selected from the group consisting of deuterium, halogen, amino, cyano, hydroxy, C₁₋₃ alkyl, C₁₋₆ alkoxy, C₁₋₃ alkylthio, C₁₋₃ haloalkyl, C₁₋₃ hydroxyalkyl, C₃₋₆ cycloalkyl and 4-6 membered heterocyclyl containing 1, 2 or 3 heteroatoms selected from N, O or S;
preferably, R₉ is selected from the group consisting of deuterium, halogen, amino, cyano, hydroxy, methyl, ethyl, cyclopropyl, cyclobutyl.

25. The compound of claim 19, or a tautomer, cis- or trans-isomer, mesomer, racemate, enantiomer, diastereomer, or mixture thereof, or a pharmaceutically acceptable salt, solvate, or prodrug thereof, wherein the compound is of formula (II):

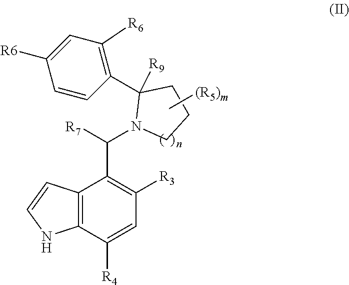

26. The compound of claim 19, or a tautomer, cis- or trans-isomer, mesomer, racemate, enantiomer, diastereomer, or mixture thereof, or a pharmaceutically acceptable salt, solvate, or prodrug thereof, wherein the compound is of formula (III-a)-(III-b): 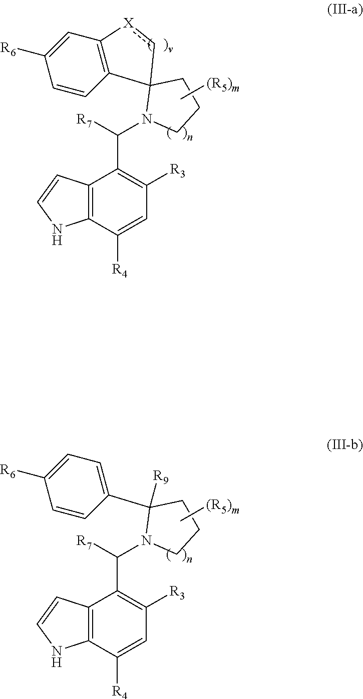
wherein,
X is CR_{c}R_{d}, NRₑ or O; [symbol]
is a single or double bond;
R_{c}, R_{d} and Rₑ are independently selected from the group consisting of hydrogen, deuterium, halogen, amino, cyano, hydroxy, C₁₋₆ alkyl, C₁₋₆ alkoxy, C₁₋₆ alkylthio, C₁₋₆ haloalkyl, C₁₋₆ hydroxyalkyl, C₃₋₈ cycloalkyl, C₃₋₈ heterocyclyl, C₆₋₁₀ aryl and C₆₋₁₀ heteroaryl;
preferably, R_{c}, R_{d} and Rₑ are independently selected from the group consisting of hydrogen, deuterium, halogen, amino, cyano, hydroxy, C₁₋₃ alkyl, C₁₋₃ alkoxy, C₁₋₃ alkylthio, C₁₋₃ haloalkyl, C₁₋₃ hydroxyalkyl, C₃₋₆ cycloalkyl, C₄-6 heterocyclyl, C₆₋₁₀ aryl and C₆₋₁₀ heteroaryl;
preferably, R_{c}, R_{d} and Rₑ are independently selected from the group consisting of hydrogen, deuterium, halogen, amino, cyano, hydroxy, methyl, ethyl, cyclopropyl, cyclobutyl;
v is 0, 1, 2 or 3.

27. The compound of claim 19, or a tautomer, cis- or trans-isomer, mesomer, racemate, enantiomer, diastereomer, or mixture thereof, or a pharmaceutically acceptable salt, solvate, or prodrug thereof, wherein the compound is of formula (II-a)-(II-b): 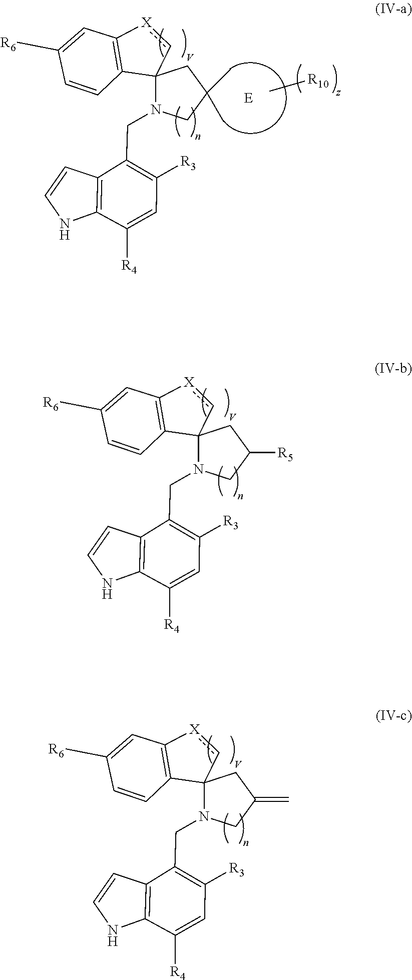 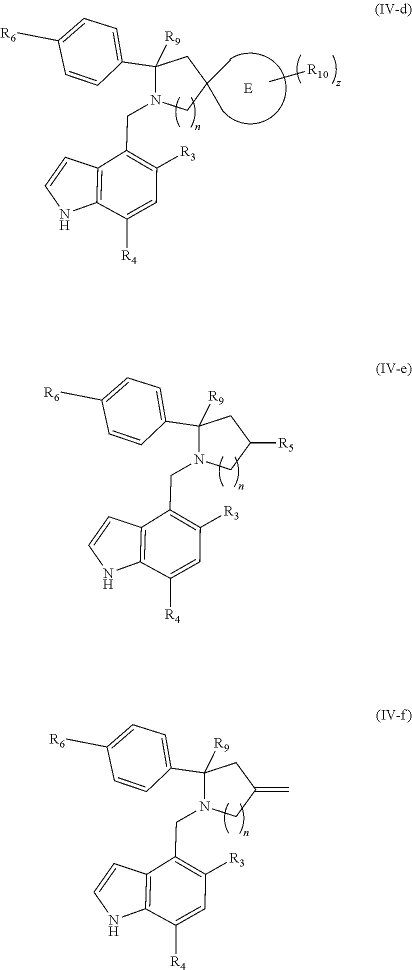
wherein,
E is 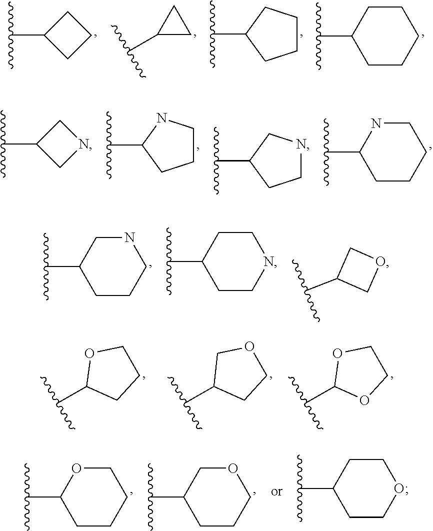
R₁₀ is selected from hydrogen, deuterium, halogen, amino, cyano, hydroxy, C₁₋₃ alkyl, C₂₋₄ alkenyl, C₂₋₄ alkynyl, C₁₋₃ alkoxy, C₁₋₃alkylthio, C₁₋₃ haloalkyl and C₁₋₃ hydroxyalkyl;
n is 2; and,
z is 0, 1, 2 or 3.

28. The compound of claim 26, or a tautomer, cis- or trans-isomer, mesomer, racemate, enantiomer, diastereomer, or mixture thereof, or a pharmaceutically acceptable salt, solvate, or prodrug thereof, wherein

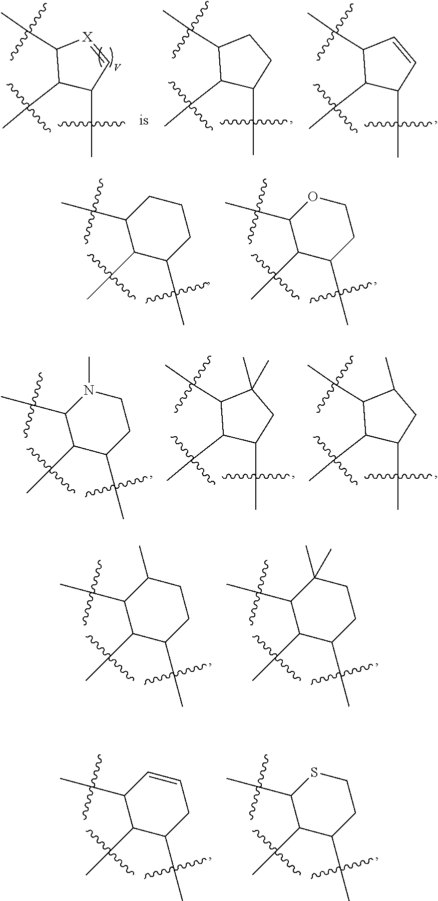

29. The compound of claim 27, or a tautomer, cis- or trans-isomer, mesomer, racemate, enantiomer, diastereomer, or mixture thereof, or a pharmaceutically acceptable salt, solvate, or prodrug thereof, wherein

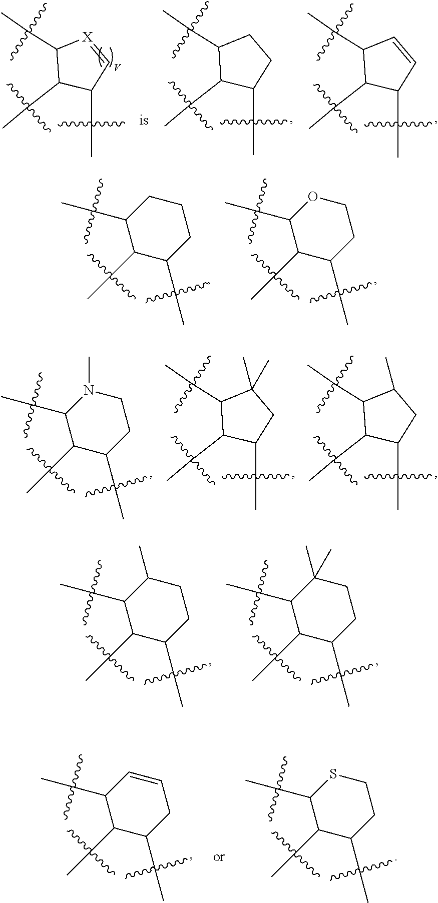

30. The compound of claim 19, or a tautomer, cis- or trans-isomer, mesomer, racemate, enantiomer, diastereomer, or mixture thereof, or a pharmaceutically acceptable salt, solvate, or prodrug thereof, wherein the compound is of formula (V-a)-(V-b): 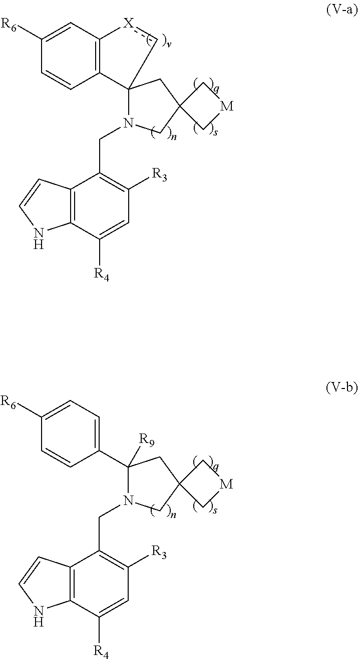
wherein,
M is CR_{f}R_{g};
R_{f} and R_{g} are independently selected from hydrogen, halogen and C₁₋₃ alkyl;
preferably, R_{f} and R_{g} are independently selected from hydrogen and fluorine;
q is 1, 2 or 3; and,
s is 0, 1 or 2.

31. The compound of claim 19, or a tautomer, cis- or trans-isomer, mesomer, racemate, enantiomer, diastereomer, or mixture thereof, or a pharmaceutically acceptable salt, solvate, or prodrug thereof, wherein the compound is of formula (VI-a)-(VI-l):

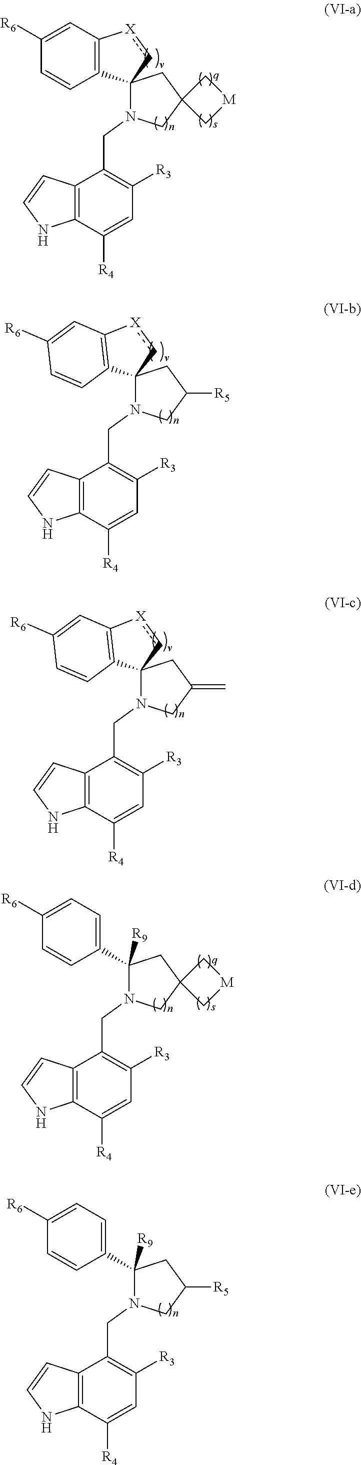

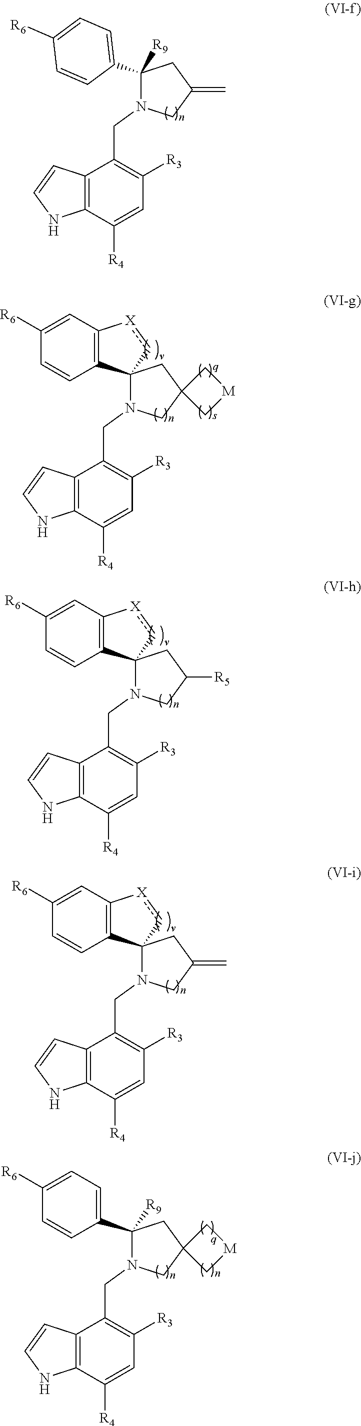

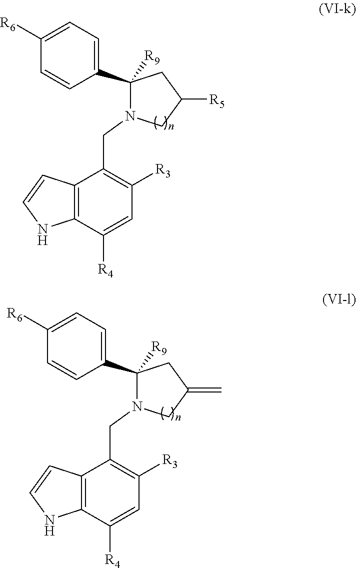

32. The compound of claim 31, or a tautomer, cis- or trans-isomer, mesomer, racemate, enantiomer, diastereomer, or mixture thereof, or a pharmaceutically acceptable salt, solvate, or prodrug thereof, wherein,

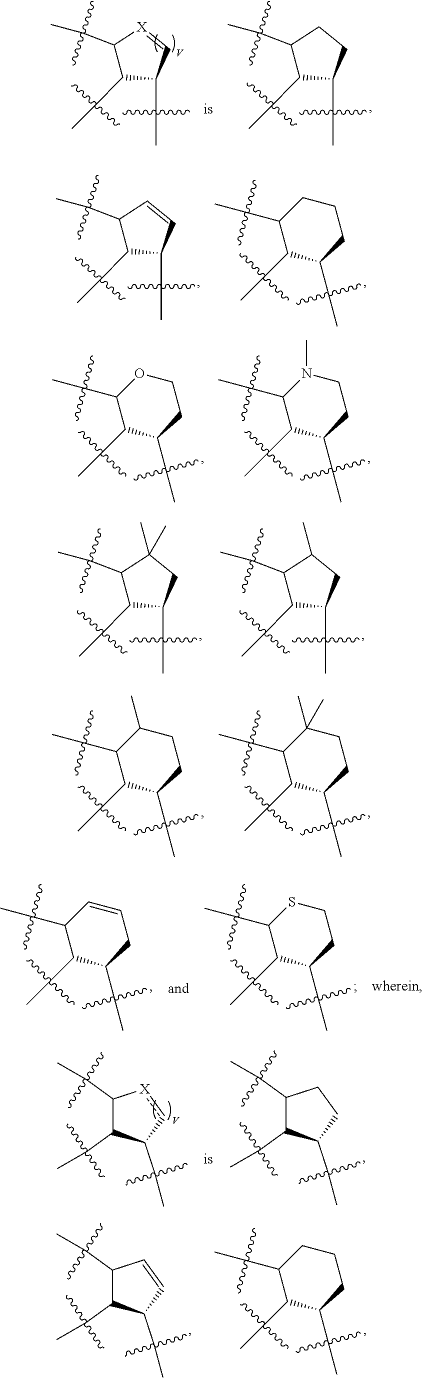

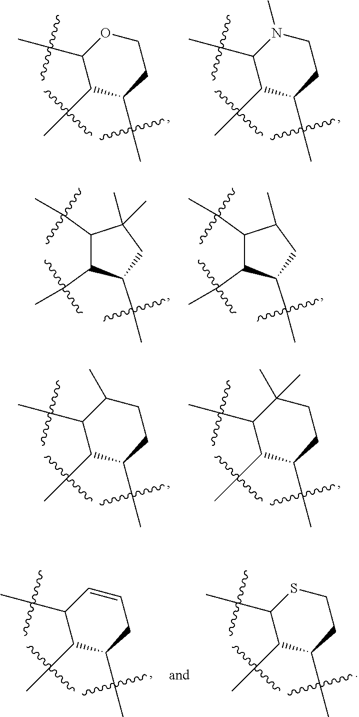

33. A compound selected from the group consisting of: 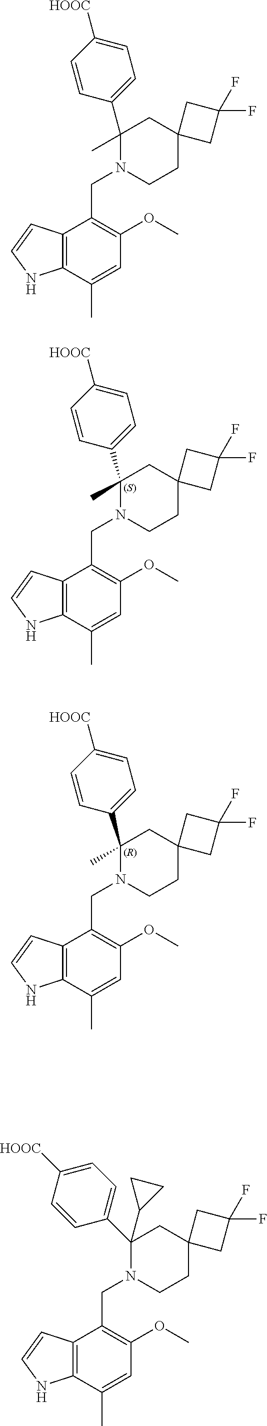 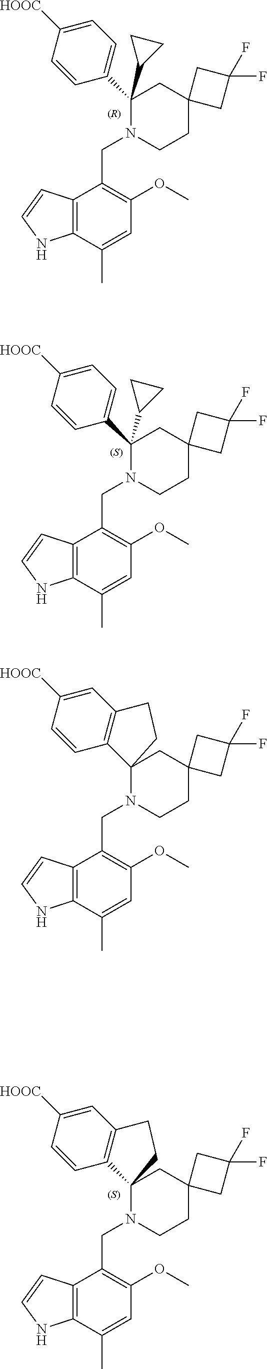 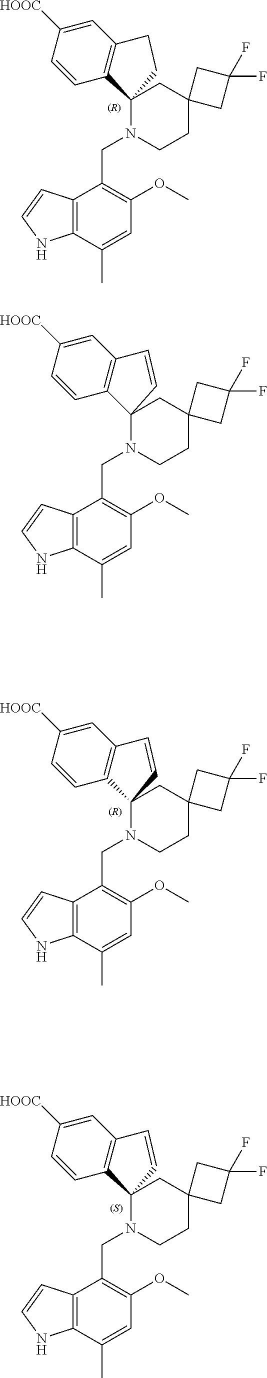 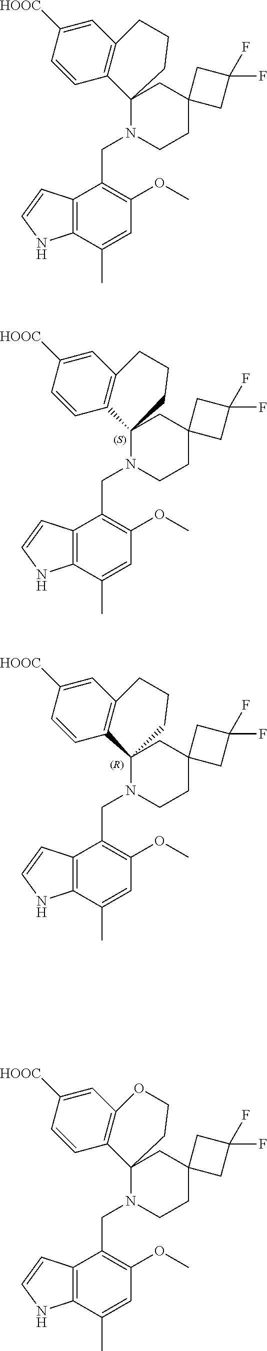 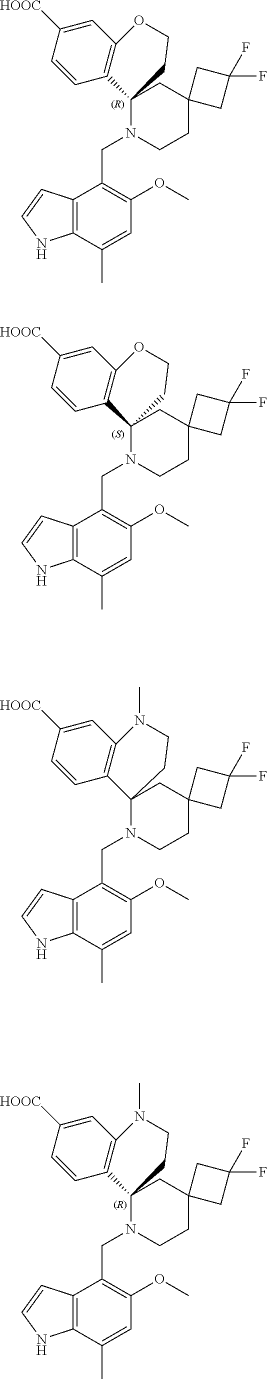 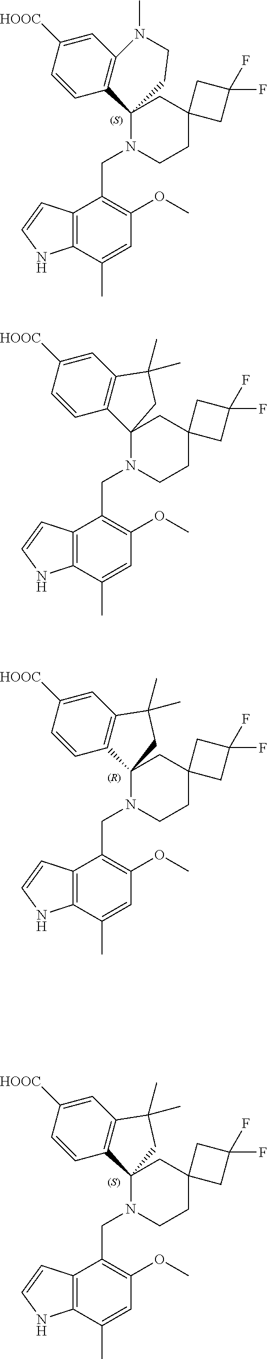 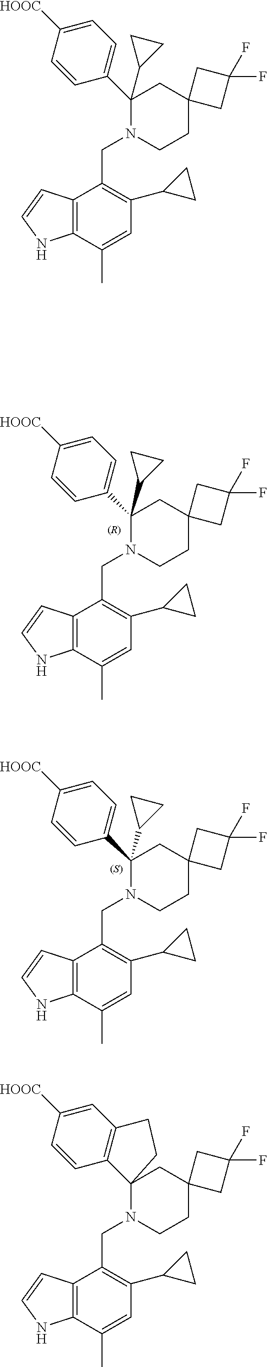 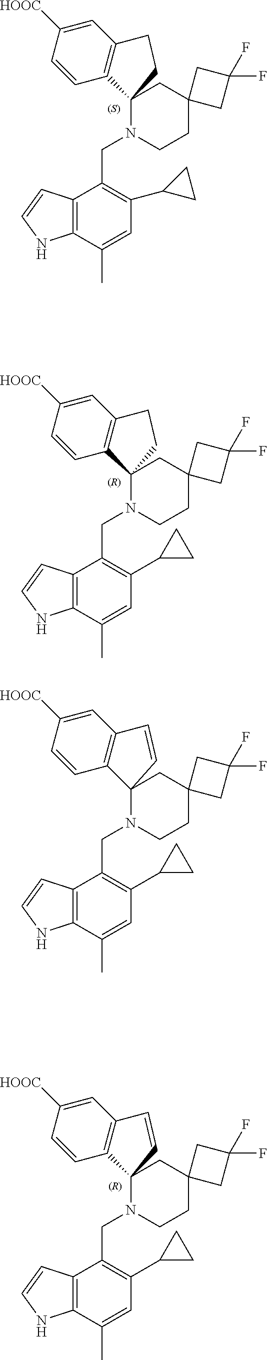 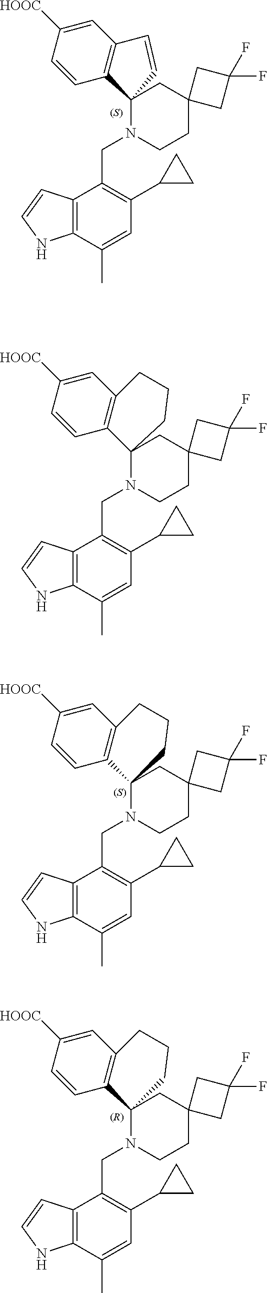 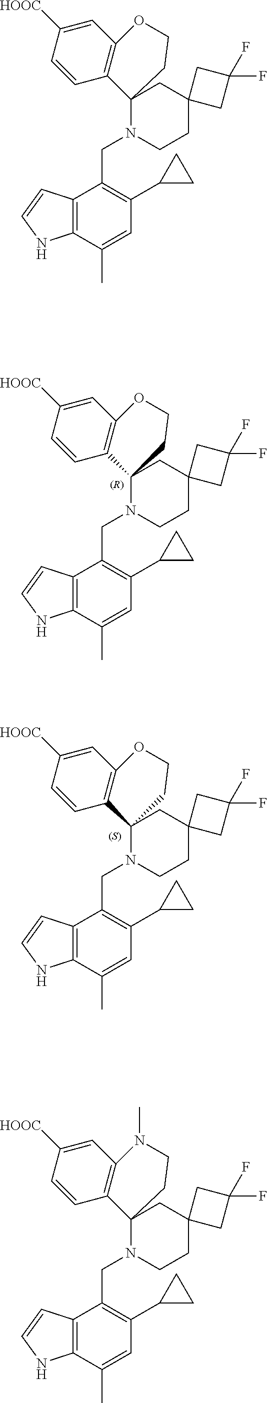 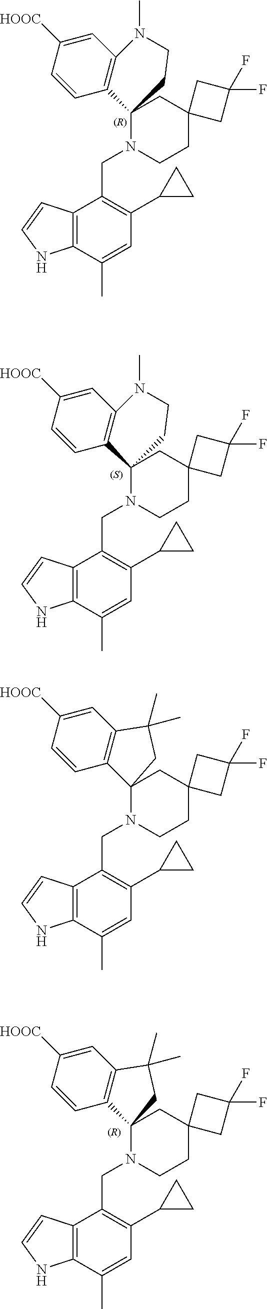 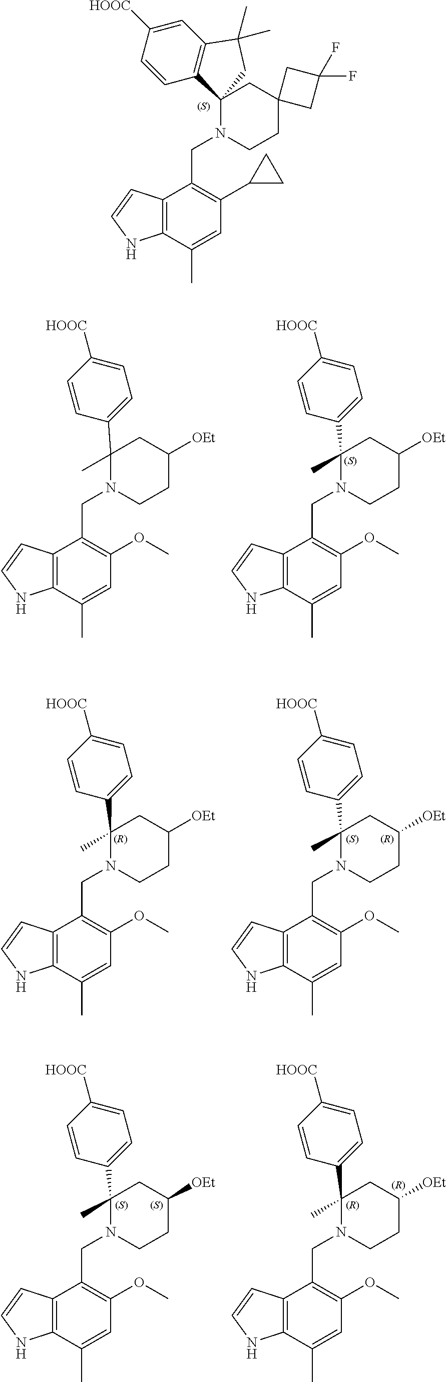 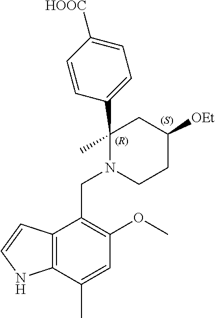 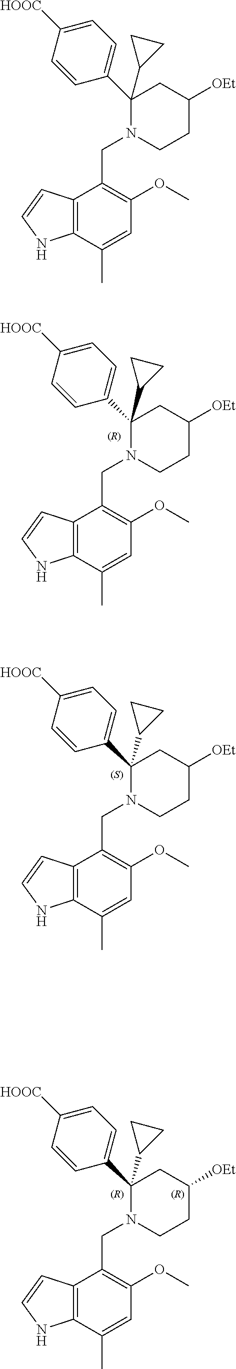 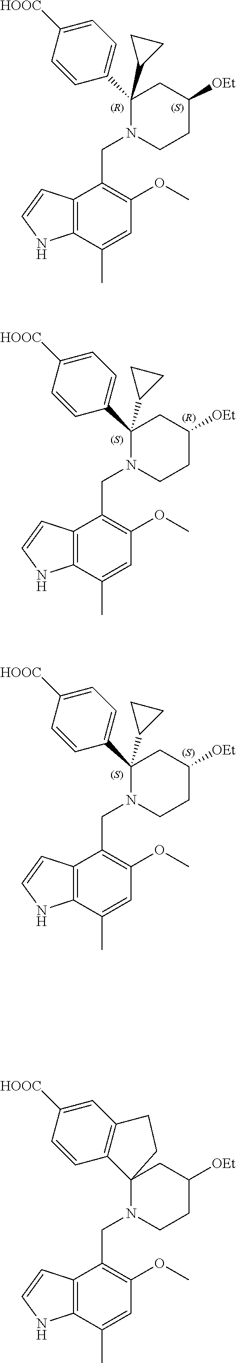 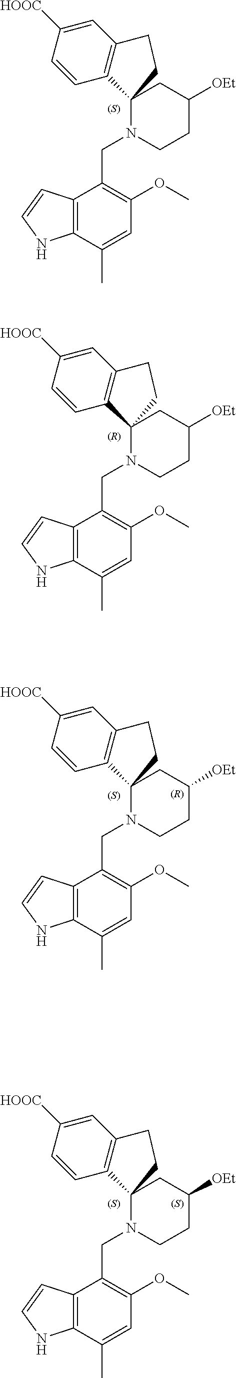 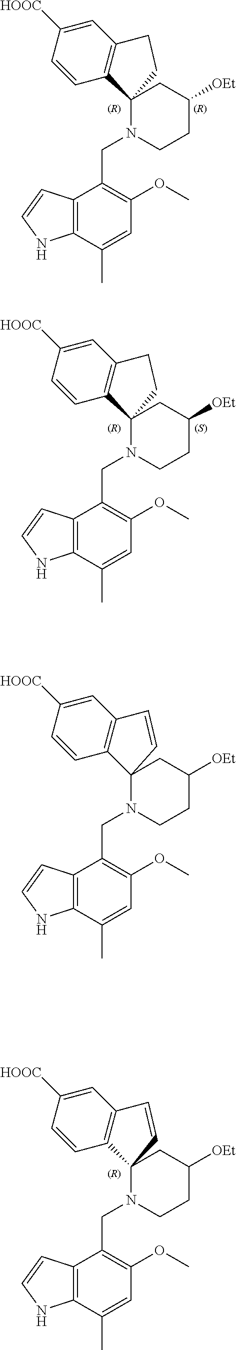 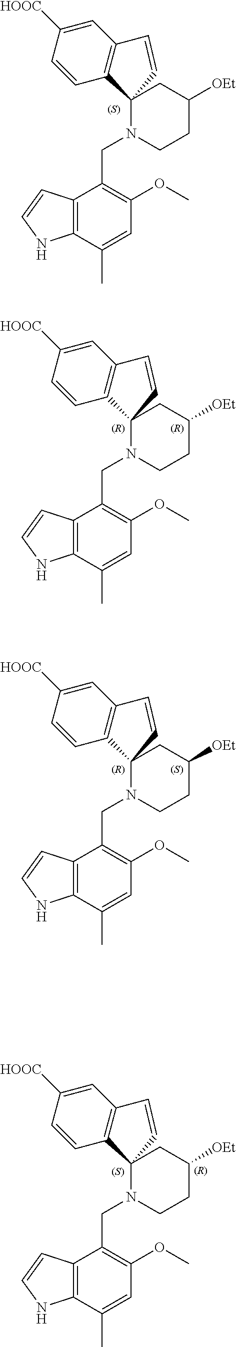 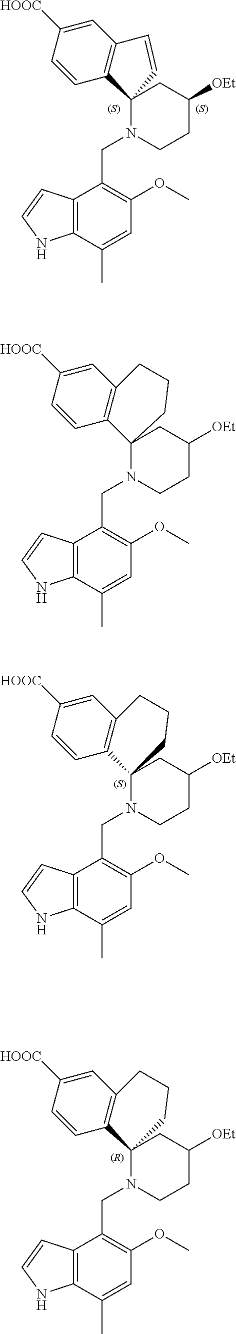 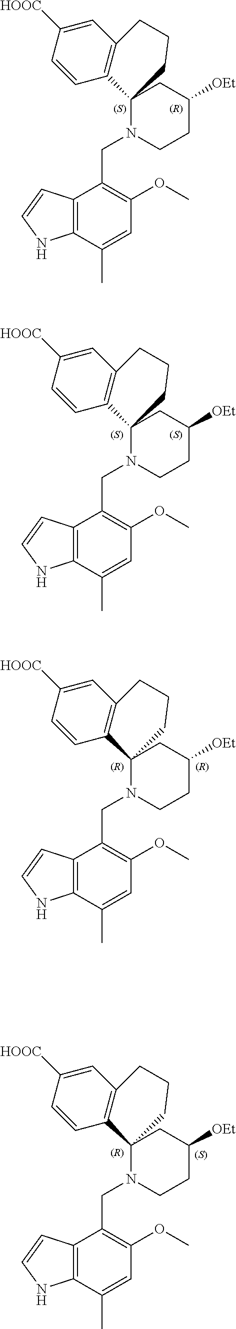 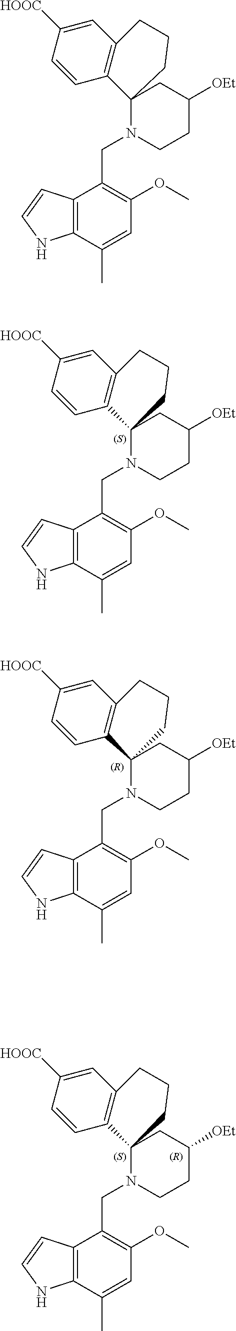 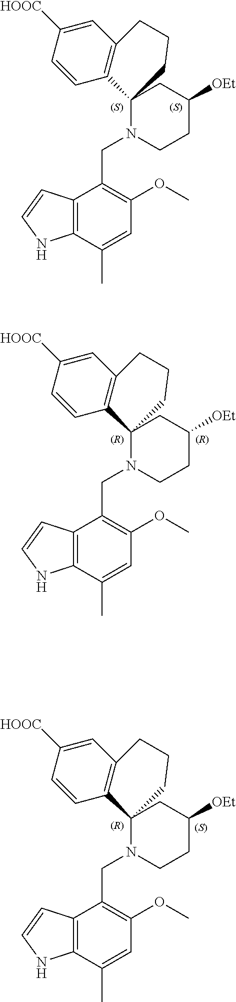 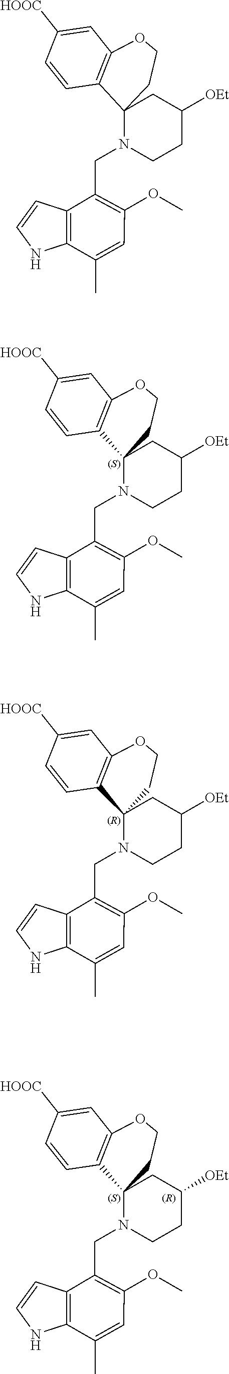 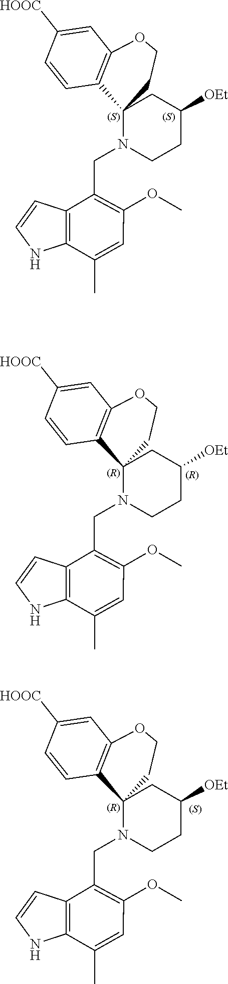 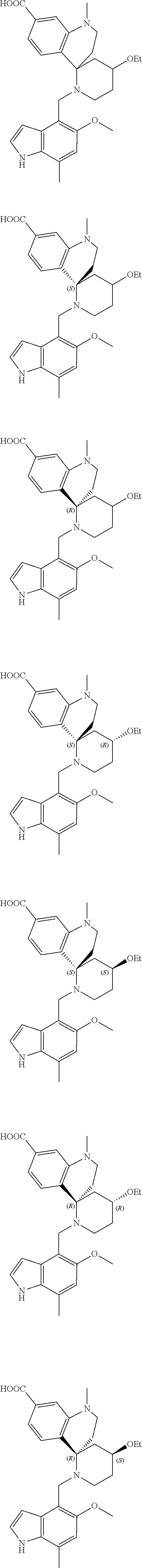 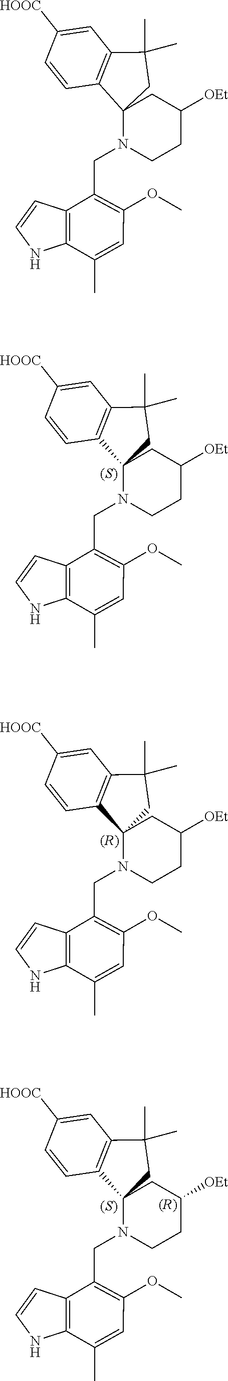 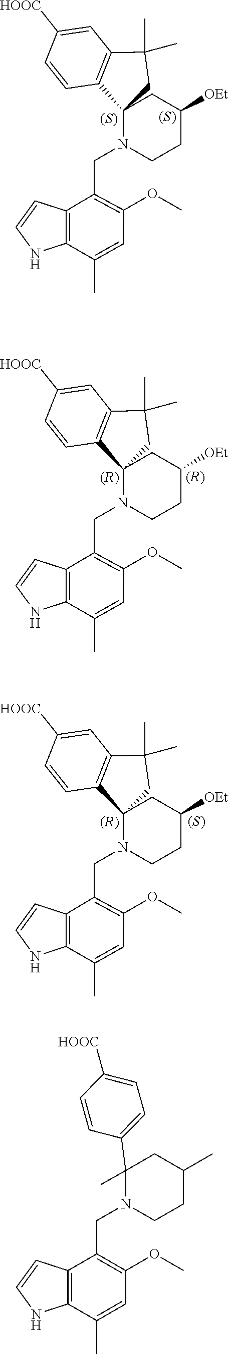 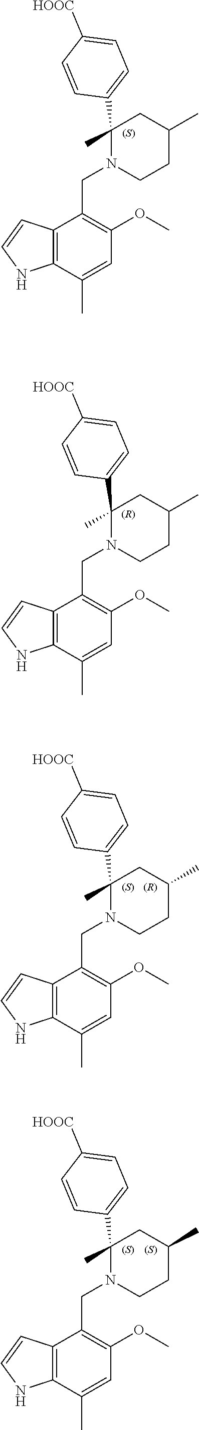 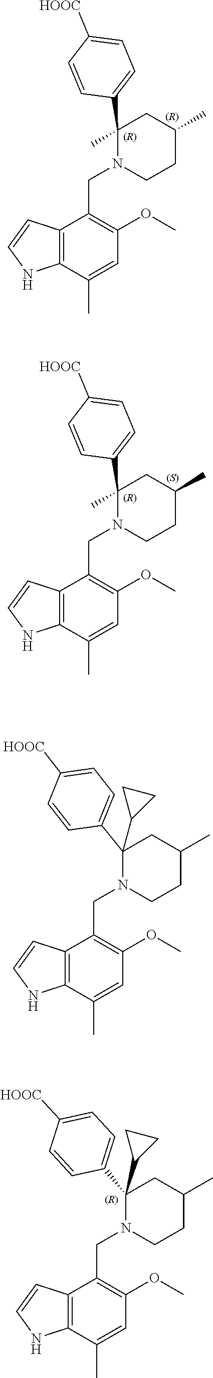 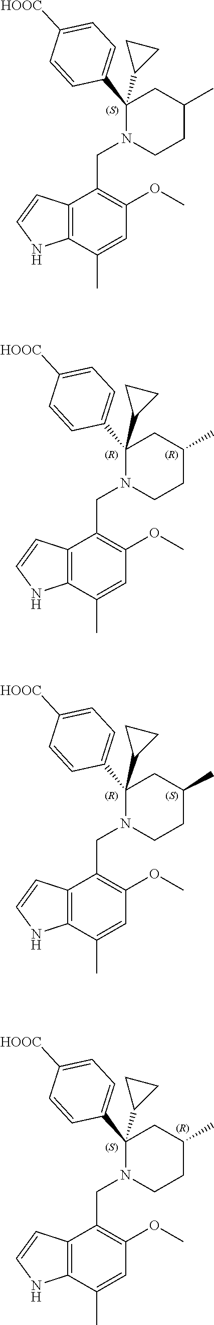 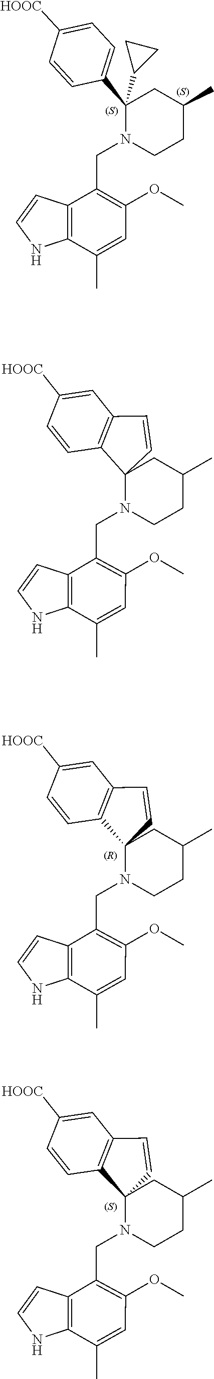 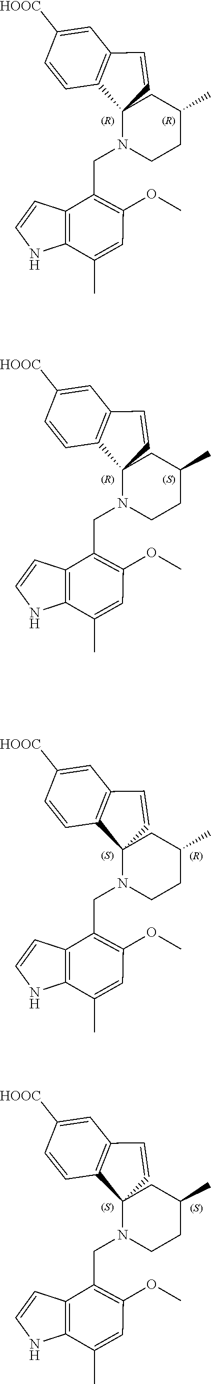 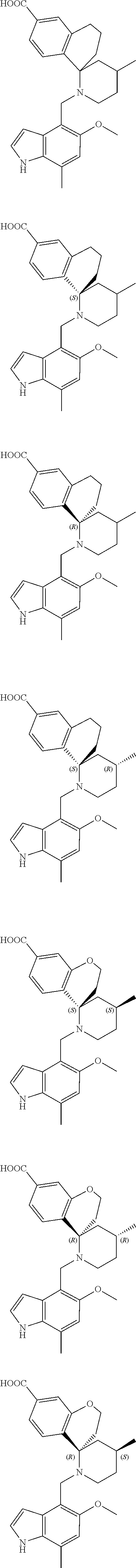 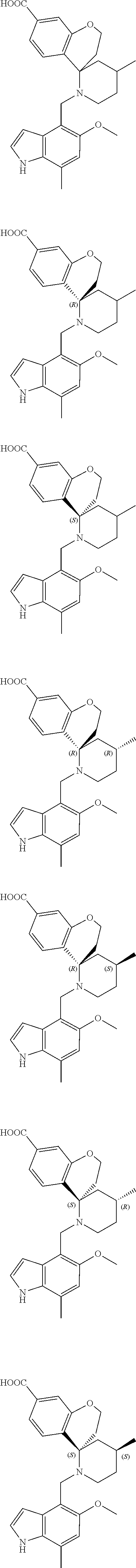 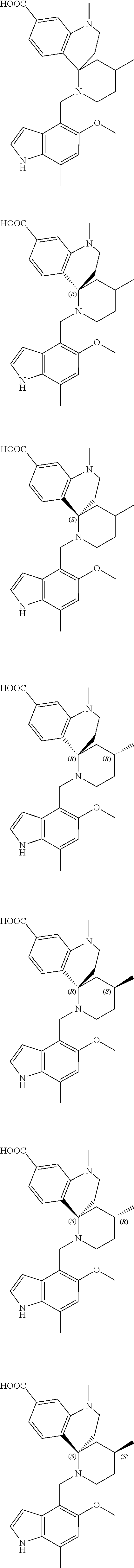 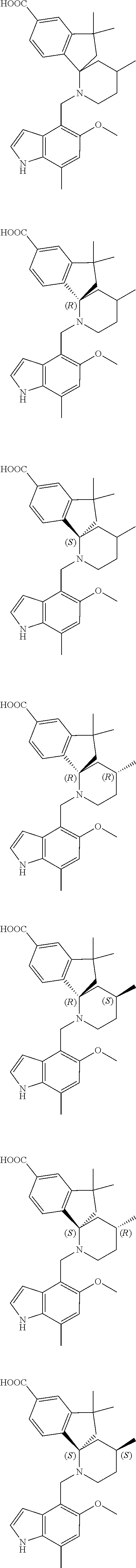 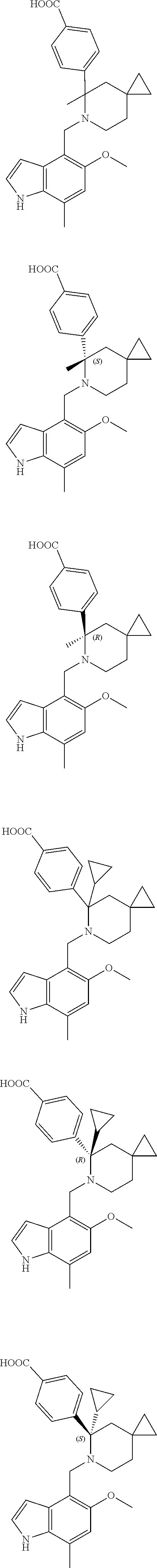 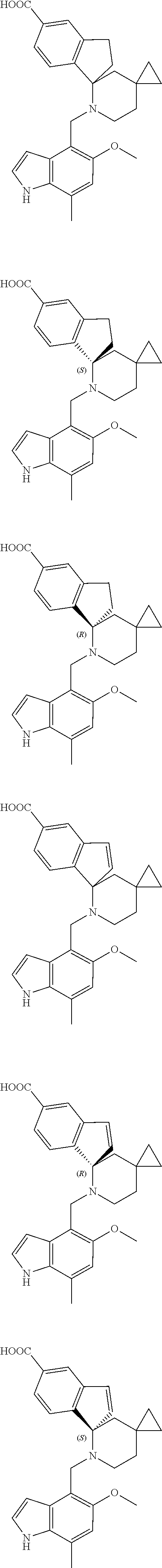 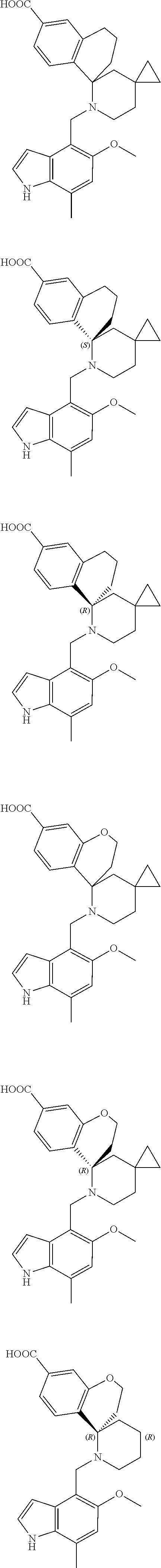 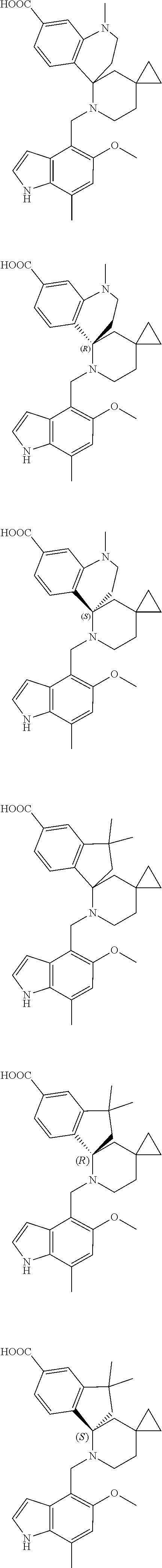 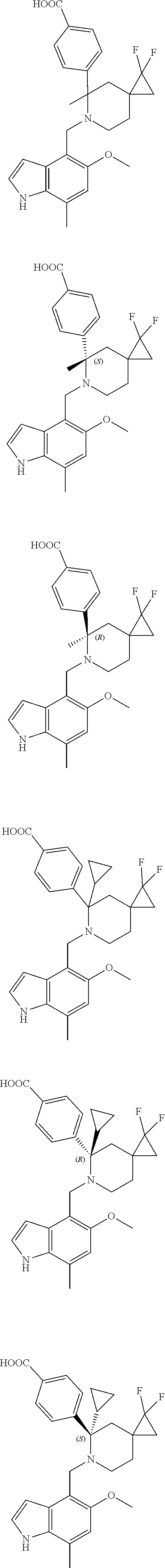 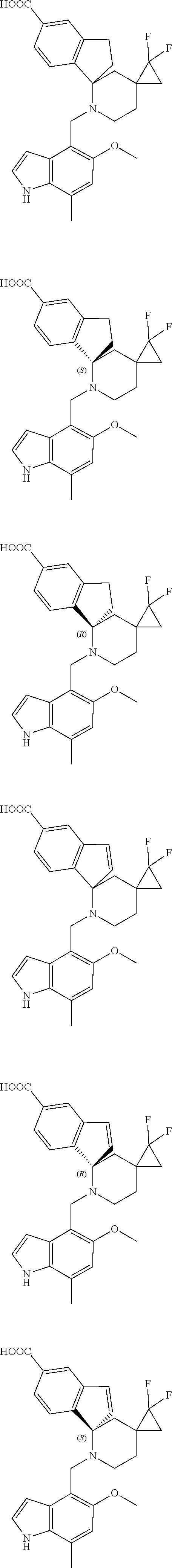 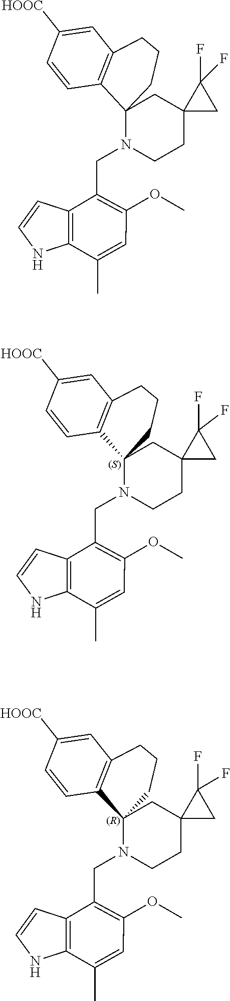 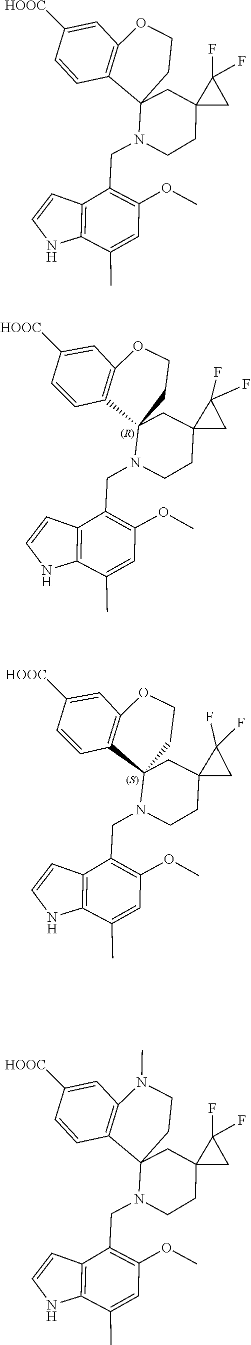 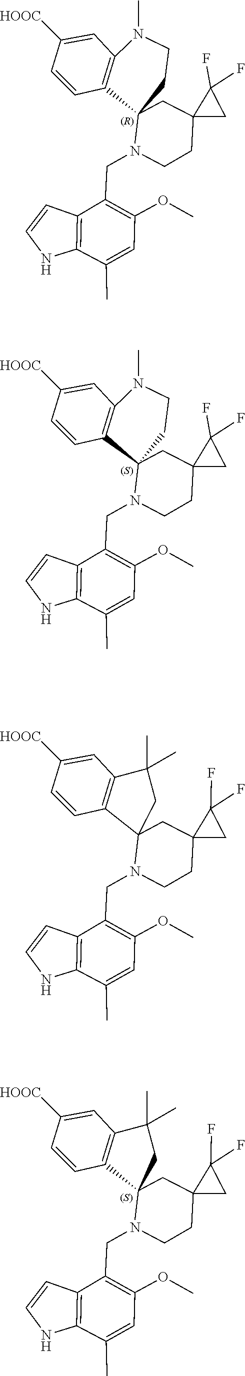 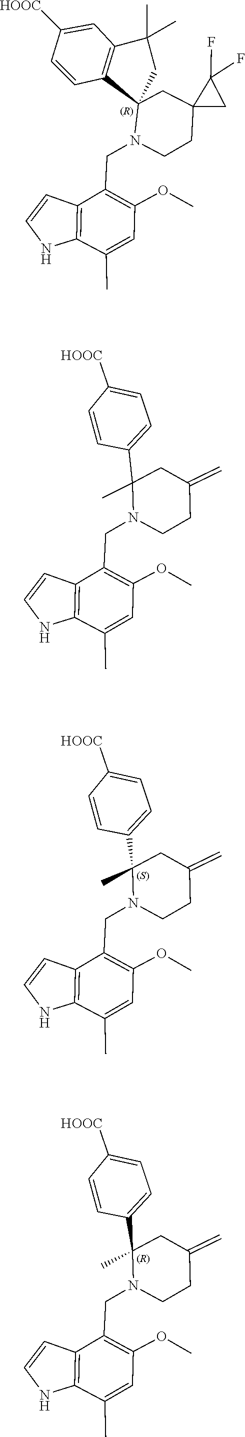 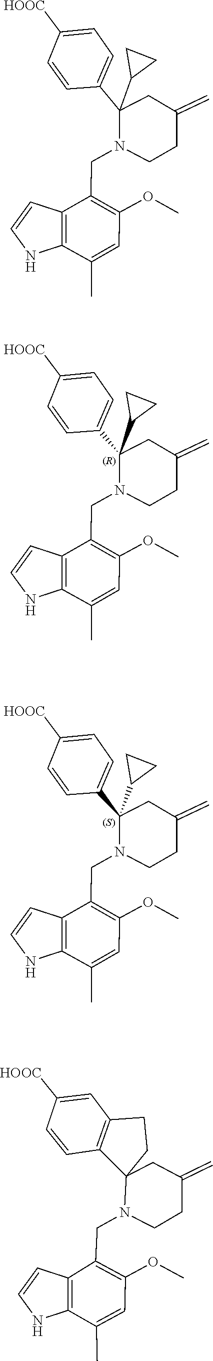 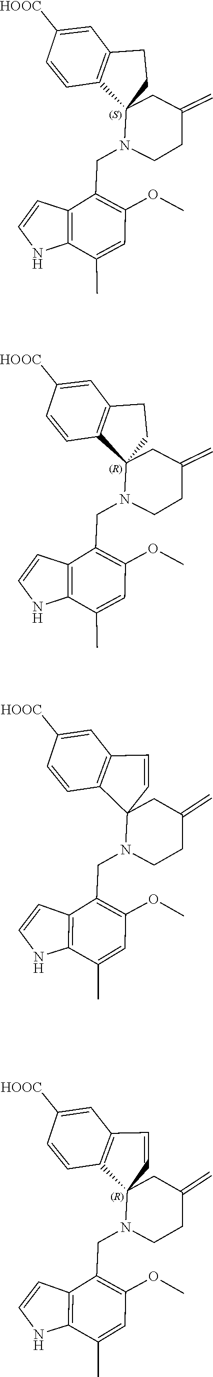 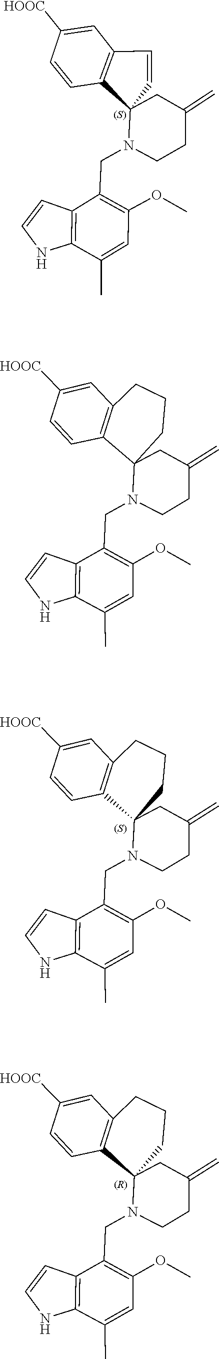 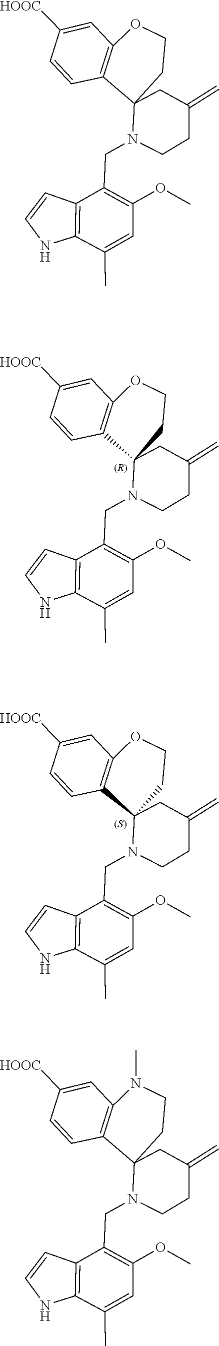 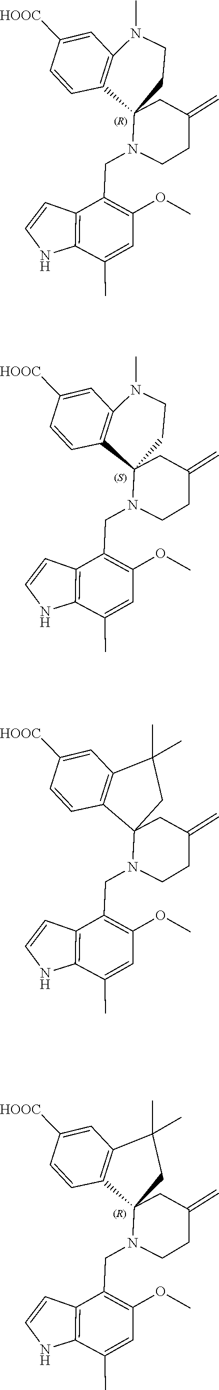 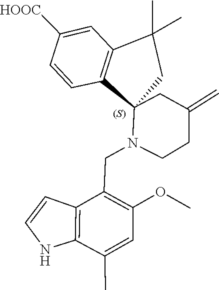
or a tautomer, cis- or trans-isomer, mesomer, racemate, enantiomer, diastereomer, or mixture thereof, or a pharmaceutically acceptable salt, solvate, or prodrug thereof.

34. A pharmaceutical composition comprising a therapeutically effective amount of the compound of claim 19, or tautomer, pharmaceutically acceptable salt thereof, and a pharmaceutically acceptable carrier or excipient.

35. A method for modulating complement alternative pathway activity in a subject, wherein the method comprises administering to the subject a therapeutically effective amount of the compound according to claim 19.

36. A method for treating a disorder or a disease in a subject mediated by complement activation, in particular mediated by activation of the complement alternative pathway, wherein the method comprises administering to the subject a therapeutically effective amount of the compound according to claim 19.

37. The method of claim 36, in which the disease or disorder is selected from the group consisting of age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus, SLE nephritis, proliferative nephritis, liver fibrosis, hemolytic anemia, myasthenia gravis, tissue regeneration, neural regeneration, dyspnea, hemoptysis, ARDS, asthma, chronic obstructive pulmonary disease, emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, glomerulonephritis and obesity.
